(19) United States
(12) Patent Application Publication
(10) Pub. No.: US 2011/0257156 A1
(43) Pub. Date: Oct. 20, 2011

(54) GAMMA SECRETASE MODULATORS

(76) Inventors: Zhaoning Zhu, Plainsboro, NJ (US); William J. Greenlee, Teaneck, NJ (US); David James Cole, Springfield, NJ (US); Dmitri A. Pissarnitski, Scotch Plains, NJ (US); Gioconda V. Gallo, Summit, NJ (US); Hongmei Li, Warren, NJ (US); Hubert B. Josien, Jersey City, NJ (US); Jun Qin, Edison, NJ (US); Chad E. Knutson, Garwood, NJ (US); Mihirbaran Mandal, Scotch Plains, NJ (US); Monica L. Vicarel, North Brunswick, NJ (US); Murali Rajagopalan, Edison, NJ (US); Pawan Kumar Dhondi, Elizabeth, NJ (US); Ruo Xu, Watchung, NJ (US); Zhong-Yue Sun, Parlin, NJ (US); Thomas A. Bara, Linden, NJ (US); Xianhai Huang, Warren, NJ (US); Xiaohong Zhu, Edison, NJ (US); Zhiqiang Zhao, Scotch Plains, NJ (US); John W. Clader, Milton, VT (US); Anandan Palani, Bridgewater, NJ (US); Theodros Asberom, West Orange, NJ (US); Troy McCracken, Garwood, NJ (US); Chad E. Bennett, Metuchen, NJ (US)

(21) Appl. No.: 13/126,056

(22) PCT Filed: Nov. 5, 2009
(86) PCT No.: PCT/US09/63396

§ 371 Date: Jul. 6, 2011

Related U.S. Application Data
(60) Provisional application No. 61/111,829, filed on Nov. 6, 2008.

(51) Int. Cl. A61K 31/55; A61K 31/5383; A61K 31/5377; A61K 31/501; A61K 31/497; A61K 31/506; A61K 31/438; A61K 31/437; C07D 413/10; C07D 498/20; C07D 498/04; A61P 25/00; A61P 25/28; A61K 31/4245; A61K 31/5386

(52) U.S. Cl. 514/210.21; 514/214.02; 514/229.5; 514/230.2; 514/230.5; 514/233.2; 514/252.04; 514/253.04; 514/255.05; 514/256; 514/278; 514/302; 514/364; 540/578; 544/70; 544/101; 544/105; 544/127; 544/238; 544/333; 544/362; 544/405; 546/15; 546/115; 548/131

(57) ABSTRACT

In its many embodiments, the present invention provides novel heterocyclic compounds as modulators of gamma secretase, methods of preparing such compounds, pharmaceutical compositions containing one or more such compounds, methods of preparing pharmaceutical formulations comprising one or more such compounds, and methods of treatment, prevention, inhibition, or amelioration of one or more diseases associated with the central nervous system using such compounds or pharmaceutical compositions.

### REFERENCE TO RELATED APPLICATION

This application claims the benefit of U.S. Provisional Application Ser. No. 61/111,829 filed Nov. 6, 2008.

### FIELD OF THE INVENTION

The present invention relates to certain heterocyclic compounds useful as gamma secretase modulators (including inhibitors, antagonists and the like), pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100 (18), p. 10417-22, suggest a molecular basis for reversible memory loss.

Nitsch R M, and 16 others, Antibodies against β-amyloid slow cognitive decline in Alzheimer's disease, Neuron, May 22, 2003, 38 (4), p. 547-554) suggest that the main components of Aβ protein are Aβ40 Consisting of 40 amino acids and Aβ42 having two additional amino acids at the C-terminal. The Aβ40 and Aβ42 tend to aggregate (for example, see Jarrell J T at al, The carboxy terminus of the β amyloid protein is critical for the seeding of amyloid formation: implications for the pathogenesis of Alzheimer's disease, Biochemistry, May 11, 1993, 32 (18), p. 4693-4697) and constitute main components of senile plaques (for example, (Glenner G G, et al, Alzheimer's disease: initial report of the purification and characterization of a novel cerebrovascular amyloid protein, Biochemical and Biophysical Research Communications, May 16, 1984, 120 (3), p. 885-90. See also Masters C L, et al, Amyloid plaque core protein in Alzheimer disease and Down syndrome, Proceeding National Academy of Science USA, June 1985, 82 (12), p. 4245-4249.).

Furthermore, it is known that mutations of APP and presenelin genes, which is observed in familial Alzheimer's disease, increase production of Aβ40 and Aβ42 (for example, see Gouras G K, et al, Intraneuronal Aβ 142 accumulation in human brain, American Journal of Pathology, January 2000, 156 (1), p. 15-20. Also, see Scheuner D, et al, Nature Medicine, August 1996, 2 (8), p. 864-870; and Forman M S, et al, Differential effects of the Swedish mutant amyloid precursor protein on β-amyloid accumulation and secretion in neurons and nonneuronal cells, Journal of Biological Chemistry, Dec. 19, 1997, 272 (51), p. 32247-32253.). Therefore, compounds which reduce production of Aβ40 and Aβ42 are expected as an agent for controlling progress of Alzheimer's disease or for preventing the disease.

These Aβs are produced when APP is cleaved by beta secretase and subsequently clipped by gamma secretase. In consideration of this, creation of inhibitors of γ secretase and β secretase has been attempted for the purpose of reducing production of Aβs. Many of these secretase inhibitors already known are peptides or peptidomimetics such as L-685,458. L-685,458, an aspartyl protease transition stale mimic, is a potent inhibitor of amyloid β-protein precursor γ-secretase activity, Biochemistry, Aug. 1, 2000, 39 (30), p. 8698-8704).

Also of interest in connection with the present invention are: US 2006/0004013 (Eisai, published Jan. 5, 2006); WO 2005/110422 (Boehringer Ingelheim, published Nov. 24, 2005); WO 2006/045554 (CellZome AG, published May 4, 2006); WO 2004/110350 (Neurogenetics, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of heterocyclic compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

Thus, this invention provides compounds selected from the group consisting of the compounds of Group A, or a pharmaceutically acceptable salt, solvate, ester or prodrug thereof.

Group A represents: compounds of formulas P2, Q3, R2, S3, T2, U2, V8, W6 (e.g., W6-1 and W6-2), X2, X3, Y2, Z2, AA2, AA3, AB2, AC12, AD7, AE4, AG2, AH7, AI2, AJ12, 201-214, 216-266, 268-424, 437-465, and 468-533.

This invention also provides compounds selected from the group consisting of the compounds of Group A.

The present invention further includes the compounds of Group A in all their isolated forms.

This invention also provides compounds selected from the group consisting of the compounds of Group A in pure and isolated form.

This invention also provides pharmaceutical compositions comprising an effective amount of one or more (e.g., one) compounds selected from the group consisting of Group A, or a pharmaceutically acceptable salt, ester or solvate thereof, and a pharmaceutically acceptable carrier.

This invention also provides pharmaceutical compositions comprising an effective amount of one or more (e.g., one) compounds selected from the group consisting of the compounds of Group A, or a pharmaceutically acceptable salt, ester or solvate thereof, and an effective amount of one or more (e.g., one) other pharmaceutically active ingredients (e.g., drugs), and a pharmaceutically acceptable carrier.

The compounds selected from the group consisting of the compounds of Group A can be useful as gamma secretase modulators and can be useful in the treatment and prevention of diseases such as, for example, central nervous system disorders such as Alzheimers disease and Downs Syndrome.

Thus, this invention also provides methods for: (1) method for modulating (including inhibiting, antagonizing and the like) gamma-secretase; (2) treating one or more neurodegenerative diseases; (3) inhibiting the deposition of amyloid protein (e.g., amyloid beta protein) in, on or around neurological tissue (e.g., the brain); (4) Alzheimer's disease; and (5) treating Downs syndrome; wherein each method comprises administering an effective amount of one or more (e.g., one) compounds selected from the group consisting of the compounds of Group A to a patient in need of such treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds selected from the group consisting of the compounds of Group A and the administration of an effective amount of one or more (e.g., one) other pharmaceutical active ingredients (e.g., drugs).

This invention also provides methods for: (1) treating mild cognitive impairment; (2) treating glaucoma; (3) treating cerebral amyloid angiopathy; (4) treating stroke; (5) treating dementia; (6) treating microgliosis; (7) treating brain inflammation; and (8) treating olfactory function loss; wherein each method comprises administering an effective amount of one or more (e.g., one) compounds selected from the group consisting of the compounds of Group A to a patient in need of such treatment.

This invention also provides a kit comprising, in separate containers, in a single package, pharmaceutical compositions for use in combination, wherein one container comprises an effective amount of a compound, selected from the group consisting of the compounds of Group A, in a pharmaceutically acceptable carrier, and another container (i.e., a second container) comprises an effective amount of another pharmaceutically active ingredient (as described below), the combined quantities of the compound of Group A and the other pharmaceutically active ingredient being effective to treat the diseases or conditions mentioned in any of the above methods.

### DETAILED DESCRIPTION

In one embodiment, the present invention discloses the compounds below, or a pharmaceutically acceptable salt, solvate, ester or prodrug thereof.

One embodiment of this invention is directed to compounds selected from the group consisting of the compounds of Group A, or a pharmaceutically acceptable salt, solvate, ester or prodrug thereof.

Group A means the compounds of formulas P2, Q3, R2, S3, T2, U2, V8, W6 (e.g., W6-1 and W6-2), X2, X3, Y2, Z2, AA2, AA3, AB2, AC12, AD7, AE4, AG2, AH7, AI2, AJ12, 201-214, 216-266, 268-424, 437-465, and 468-533, as identified below.

Another embodiment of this invention is directed to compound P2.

Another embodiment of this invention is directed to compound Q3.

Another embodiment of this invention is directed to compound R2.

Another embodiment of this invention is directed to compound S3.

Another embodiment of this invention is directed to compound T2.

Another embodiment of this invention is directed to compound U2.

Another embodiment of this invention is directed to compound V8.

Another embodiment of this invention is directed to compound W6.

Another embodiment of this invention is directed to compound W6-1.

Another embodiment of this invention is directed to compound W6-2.

Another embodiment of this invention is directed to compound X2.

Another embodiment of this invention is directed to compound X3.

Another embodiment of this invention is directed to compound Y2.

Another embodiment of this invention is directed to compound Z2.

Another embodiment of this invention is directed to compound AA2.

Another embodiment of this invention is directed to compound AA3.

Another embodiment of this invention is directed to compound AB2.

Another embodiment of this invention is directed to compound AC12.

Another embodiment of this invention is directed to compound AD7.

Another embodiment of this invention is directed to compound AE4.

Another embodiment of this invention is directed to compound AG2.

Another embodiment of this invention is directed to compound AH7.

Another embodiment of this invention is directed to compound AI2.

Another embodiment of this invention is directed to compound AJ12.

Another embodiment of this invention is directed to compound 201.

Another embodiment of this invention is directed to compound 202.

Another embodiment of this invention is directed to compound 203.

Another embodiment of this invention is directed to compound 204.

Another embodiment of this invention is directed to compound 205.

Another embodiment of this invention is directed to compound 206.

Another embodiment of this invention is directed to compound 207.

Another embodiment of this invention is directed to compound 208.

Another embodiment of this invention is directed to compound 209.

Another embodiment of this invention is directed to compound 210.

Another embodiment of this invention is directed to compound 211.

Another embodiment of this invention is directed to compound 212.

Another embodiment of this invention is directed to compound 213.

Another embodiment of this invention is directed to compound 214.

Another embodiment of this invention is directed to compound 216.

Another embodiment of this invention is directed to compound 217.

Another embodiment of this invention is directed to compound 218.

Another embodiment of this invention is directed to compound 219.

Another embodiment of this invention is directed to compound 220.

Another embodiment of this invention is directed to compound 221.

Another embodiment of this invention is directed to compound 222.

Another embodiment of this invention is directed to compound 223.

Another embodiment of this invention is directed to compound 224.

Another embodiment of this invention is directed to compound 225.

Another embodiment of this invention is directed to compound 226.

Another embodiment of this invention is directed to compound 227.

Another embodiment of this invention is directed to compound 228.

Another embodiment of this invention is directed to compound 229.

Another embodiment of this invention is directed to compound 230.

Another embodiment of this invention is directed to compound 231.

Another embodiment of this invention is directed to compound 232.

Another embodiment of this invention is directed to compound 233.

Another embodiment of this invention is directed to compound 234.

Another embodiment of this invention is directed to compound 235.

Another embodiment of this invention is directed to compound 236.

Another embodiment of this invention is directed to compound 237.

Another embodiment of this invention is directed to compound 238.

Another embodiment of this invention is directed to compound 239.

Another embodiment of this invention is directed to compound 240.

Another embodiment of this invention is directed to compound 241.

Another embodiment of this invention is directed to compound 242.

Another embodiment of this invention is directed to compound 243.

Another embodiment of this invention is directed to compound 244.

Another embodiment of this invention is directed to compound 245.

Another embodiment of this invention is directed to compound 246.

Another embodiment of this invention is directed to compound 247.

Another embodiment of this invention is directed to compound 248.

Another embodiment of this invention is directed to compound 249.

Another embodiment of this invention is directed to compound 250.

Another embodiment of this invention is directed to compound 251.

Another embodiment of this invention is directed to compound 252.

Another embodiment of this invention is directed to compound 253.

Another embodiment of this invention is directed to compound 254.

Another embodiment of this invention is directed to compound 255.

Another embodiment of this invention is directed to compound 256.

Another embodiment of this invention is directed to compound 257.

Another embodiment of this invention is directed to compound 258.

Another embodiment of this invention is directed to compound 259.

Another embodiment of this invention is directed to compound 260.

Another embodiment of this invention is directed to compound 261.

Another embodiment of this invention is directed to compound 262.

Another embodiment of this invention is directed to compound 263.

Another embodiment of this invention is directed to compound 264.

Another embodiment of this invention is directed to compound 265.

Another embodiment of this invention is directed to compound 266.

Another embodiment of this invention is directed to compound 268.

Another embodiment of this invention is directed to compound 269.

Another embodiment of this invention is directed to compound 270.

Another embodiment of this invention is directed to compound 271.

Another embodiment of this invention is directed to compound 272.

Another embodiment of this invention is directed to compound 273.

Another embodiment of this invention is directed to compound 274.

Another embodiment of this invention is directed to compound 275.

Another embodiment of this invention is directed to compound 276.

Another embodiment of this invention is directed to compound 277.

Another embodiment of this invention is directed to compound 278.

Another embodiment of this invention is directed to compound 279.

Another embodiment of this invention is directed to compound 280.

Another embodiment of this invention is directed to compound 281.

Another embodiment of this invention is directed to compound 282.

Another embodiment of this invention is directed to compound 283.

Another embodiment of this invention is directed to compound 284.

Another embodiment of this invention is directed to compound 285.

Another embodiment of this invention is directed to compound 286.

Another embodiment of this invention is directed to compound 287.

Another embodiment of this invention is directed to compound 288.

Another embodiment of this invention is directed to compound 289.

Another embodiment of this invention is directed to compound 290.

Another embodiment of this invention is directed to compound 291.

Another embodiment of this invention is directed to compound 292.

Another embodiment of this invention is directed to compound 293.

Another embodiment of this invention is directed to compound 294.

Another embodiment of this invention is directed to compound 295.

Another embodiment of this invention is directed to compound 296.

Another embodiment of this invention is directed to compound 297.

Another embodiment of this invention is directed to compound 298.

Another embodiment of this invention is directed to compound 299.

Another embodiment of this invention is directed to compound 300.

Another embodiment of this invention is directed to compound 301.

Another embodiment of this invention is directed to compound 302.

Another embodiment of this invention is directed to compound 303.

Another embodiment of this invention is directed to compound 304.

Another embodiment of this invention is directed to compound 305.

Another embodiment of this invention is directed to compound 306.

Another embodiment of this invention is directed to compound 307.

Another embodiment of this invention is directed to compound 308.

Another embodiment of this invention is directed to compound 309.

Another embodiment of this invention is directed to compound 310.

Another embodiment of this invention is directed to compound 311.

Another embodiment of this invention is directed to compound 312.

Another embodiment of this invention is directed to compound 313.

Another embodiment of this invention is directed to compound 314.

Another embodiment of this invention is directed to compound 315.

Another embodiment of this invention is directed to compound 316.

Another embodiment of this invention is directed to compound 317.

Another embodiment of this invention is directed to compound 318.

Another embodiment of this invention is directed to compound 319.

Another embodiment of this invention is directed to compound 320.

Another embodiment of this invention is directed to compound 321.

Another embodiment of this invention is directed to compound 322.

Another embodiment of this invention is directed to compound 323.

Another embodiment of this invention is directed to compound 324.

Another embodiment of this invention is directed to compound 325.

Another embodiment of this invention is directed to compound 326.

Another embodiment of this invention is directed to compound 327.

Another embodiment of this invention is directed to compound 328.

Another embodiment of this invention is directed to compound 329.

Another embodiment of this invention is directed to compound 330.

Another embodiment of this invention is directed to compound 331.

Another embodiment of this invention is directed to compound 332.

Another embodiment of this invention is directed to compound 333.

Another embodiment of this invention is directed to compound 334.

Another embodiment of this invention is directed to compound 335.

Another embodiment of this invention is directed to compound 336.

Another embodiment of this invention is directed to compound 337.

Another embodiment of this invention is directed to compound 338.

Another embodiment of this invention is directed to compound 339.

Another embodiment of this invention is directed to compound 340.

Another embodiment of this invention is directed to compound 341.

Another embodiment of this invention is directed to compound 342.

Another embodiment of this invention is directed to compound 343.

Another embodiment of this invention is directed to compound 344.

Another embodiment of this invention is directed to compound 345.

Another embodiment of this invention is directed to compound 346.

Another embodiment of this invention is directed to compound 347.

Another embodiment of this invention is directed to compound 348.

Another embodiment of this invention is directed to compound 349.

Another embodiment of this invention is directed to compound 350.

Another embodiment of this invention is directed to compound 351.

Another embodiment of this invention is directed to compound 352.

Another embodiment of this invention is directed to compound 353.

Another embodiment of this invention is directed to compound 354.

Another embodiment of this invention is directed to compound 355.

Another embodiment of this invention is directed to compound 356.

Another embodiment of this invention is directed to compound 357.

Another embodiment of this invention is directed to compound 358.

Another embodiment of this invention is directed to compound 359.

Another embodiment of this invention is directed to compound 360.

Another embodiment of this invention is directed to compound 361.

Another embodiment of this invention is directed to compound 362.

Another embodiment of this invention is directed to compound 363.

Another embodiment of this invention is directed to compound 364.

Another embodiment of this invention is directed to compound 365.

Another embodiment of this invention is directed to compound 366.

Another embodiment of this invention is directed to compound 367.

Another embodiment of this invention is directed to compound 368.

Another embodiment of this invention is directed to compound 369.

Another embodiment of this invention is directed to compound 370.

Another embodiment of this invention is directed to compound 371.

Another embodiment of this invention is directed to compound 372.

Another embodiment of this invention is directed to compound 373.

Another embodiment of this invention is directed to compound 374.

Another embodiment of this invention is directed to compound 375.

Another embodiment of this invention is directed to compound 376.

Another embodiment of this invention is directed to compound 377.

Another embodiment of this invention is directed to compound 378.

Another embodiment of this invention is directed to compound 379.

Another embodiment of this invention is directed to compound 380.

Another embodiment of this invention is directed to compound 381.

Another embodiment of this invention is directed to compound 382.

Another embodiment of this invention is directed to compound 383.

Another embodiment of this invention is directed to compound 384.

Another embodiment of this invention is directed to compound 385.

Another embodiment of this invention is directed to compound 386.

Another embodiment of this invention is directed to compound 387.

Another embodiment of this invention is directed to compound 388.

Another embodiment of this invention is directed to compound 389.

Another embodiment of this invention is directed to compound 390.

Another embodiment of this invention is directed to compound 391.

Another embodiment of this invention is directed to compound 392.

Another embodiment of this invention is directed to compound 393.

Another embodiment of this invention is directed to compound 394.

Another embodiment of this invention is directed to compound 395.

Another embodiment of this invention is directed to compound 396.

Another embodiment of this invention is directed to compound 397.

Another embodiment of this invention is directed to compound 398.

Another embodiment of this invention is directed to compound 399.

Another embodiment of this invention is directed to compound 400.

Another embodiment of this invention is directed to compound 401.

Another embodiment of this invention is directed to compound 402.

Another embodiment of this invention is directed to compound 403.

Another embodiment of this invention is directed to compound 404.

Another embodiment of this invention is directed to compound 405.

Another embodiment of this invention is directed to compound 406.

Another embodiment of this invention is directed to compound 407.

Another embodiment of this invention is directed to compound 408.

Another embodiment of this invention is directed to compound 409.

Another embodiment of this invention is directed to compound 410.

Another embodiment of this invention is directed to compound 411.

Another embodiment of this invention is directed to compound 412.

Another embodiment of this invention is directed to compound 413.

Another embodiment of this invention is directed to compound 414.

Another embodiment of this invention is directed to compound 415.

Another embodiment of this invention is directed to compound 416.

Another embodiment of this invention is directed to compound 417.

Another embodiment of this invention is directed to compound 418.

Another embodiment of this invention is directed to compound 419.

Another embodiment of this invention is directed to compound 420.

Another embodiment of this invention is directed to compound 421.

Another embodiment of this invention is directed to compound 422.

Another embodiment of this invention is directed to compound 423.

Another embodiment of this invention is directed to compound 424.

Another embodiment of this invention is directed to compound 437.

Another embodiment of this invention is directed to compound 438.

Another embodiment of this invention is directed to compound 439.

Another embodiment of this invention is directed to compound 440.

Another embodiment of this invention is directed to compound 441.

Another embodiment of this invention is directed to compound 442.

Another embodiment of this invention is directed to compound 443.

Another embodiment of this invention is directed to compound 444.

Another embodiment of this invention is directed to compound 445.

Another embodiment of this invention is directed to compound 446.

Another embodiment of this invention is directed to compound 447.

Another embodiment of this invention is directed to compound 448.

Another embodiment of this invention is directed to compound 449.

Another embodiment of this invention is directed to compound 450.

Another embodiment of this invention is directed to compound 451.

Another embodiment of this invention is directed to compound 452.

Another embodiment of this invention is directed to compound 453.

Another embodiment of this invention is directed to compound 454.

Another embodiment of this invention is directed to compound 455.

Another embodiment of this invention is directed to compound 456.

Another embodiment of this invention is directed to compound 457.

Another embodiment of this invention is directed to compound 458.

Another embodiment of this invention is directed to compound 459.

Another embodiment of this invention is directed to compound 460.

Another embodiment of this invention is directed to compound 461.

Another embodiment of this invention is directed to compound 462.

Another embodiment of this invention is directed to compound 463.

Another embodiment of this invention is directed to compound 464.

Another embodiment of this invention is directed to compound 465.

Another embodiment of this invention is directed to compound 468.

Another embodiment of this invention is directed to compound 469.

Another embodiment of this invention is directed to compound 470.

Another embodiment of this invention is directed to compound 471.

Another embodiment of this invention is directed to compound 472.

Another embodiment of this invention is directed to compound 473.

Another embodiment of this invention is directed to compound 474.

Another embodiment of this invention is directed to compound 475.

Another embodiment of this invention is directed to compound 476.

Another embodiment of this invention is directed to compound 477.

Another embodiment of this invention is directed to compound 478.

Another embodiment of this invention is directed to compound 479.

Another embodiment of this invention is directed to compound 480.

Another embodiment of this invention is directed to compound 481.

Another embodiment of this invention is directed to compound 482.

Another embodiment of this invention is directed to compound 483.

Another embodiment of this invention is directed to compound 484.

Another embodiment of this invention is directed to compound 485.

Another embodiment of this invention is directed to compound 486.

Another embodiment of this invention is directed to compound 487.

Another embodiment of this invention is directed to compound 488.

Another embodiment of this invention is directed to compound 489.

Another embodiment of this invention is directed to compound 490.

Another embodiment of this invention is directed to compound 491.

Another embodiment of this invention is directed to compound 492.

Another embodiment of this invention is directed to compound 493.

Another embodiment of this invention is directed to compound 494.

Another embodiment of this invention is directed to compound 495.

Another embodiment of this invention is directed to compound 496.

Another embodiment of this invention is directed to compound 497.

Another embodiment of this invention is directed to compound 498.

Another embodiment of this invention is directed to compound 500.

Another embodiment of this invention is directed to compound 501.

Another embodiment of this invention is directed to compound 502.

Another embodiment of this invention is directed to compound 503.

Another embodiment of this invention is directed to compound 504.

Another embodiment of this invention is directed to compound 505.

Another embodiment of this invention is directed to compound 506.

Another embodiment of this invention is directed to compound 507.

Another embodiment of this invention is directed to compound 508.

Another embodiment of this invention is directed to compound 509.

Another embodiment of this invention is directed to compound 510.

Another embodiment of this invention is directed to compound 511.

Another embodiment of this invention is directed to compound 512.

Another embodiment of this invention is directed to compound 513.

Another embodiment of this invention is directed to compound 514.

Another embodiment of this invention is directed to compound 515.

Another embodiment of this invention is directed to compound 516.

Another embodiment of this invention is directed to compound 517.

Another embodiment of this invention is directed to compound 518.

Another embodiment of this invention is directed to compound 519.

Another embodiment of this invention is directed to compound 520.

Another embodiment of this invention is directed to compound 521.

Another embodiment of this invention is directed to compound 522.

Another embodiment of this invention is directed to compound 523.

Another embodiment of this invention is directed to compound 524.

Another embodiment of this invention is directed to compound 525.

Another embodiment of this invention is directed to compound 526.

Another embodiment of this invention is directed to compound 527.

Another embodiment of this invention is directed to compound 528.

Another embodiment of this invention is directed to compound 529.

Another embodiment of this invention is directed to compound 530.

Another embodiment of this invention is directed to compound 531.

Another embodiment of this invention is directed to compound 532.

Another embodiment of this invention is directed to compound 533.

The compounds of this invention are useful for treating central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

Thus, for example, the compounds of this invention can be used to treat the following diseases or conditions: Alzheimers disease, mild cognitive impairment (MCI), Downs Syndrome, Glaucoma (Guo et. al., Proc. Natl. Acad. Sci. USA 104, 13444-13449 (2007)), Cerebral amyloid angiopathy, stroke or dementia (Frangione et al., Amyloid: J. Protein folding Disord. 8, suppl. 1, 36-42 (2001), Microgliosis and brain inflammation (M P camber, Proc. Natl. Acad. Sci. USA 95, 6448-53 (1998)), and Olfactory function loss (Getchell, et. al. Neurobiology of Aging, 663-673, 24, 2003).

In the embodiments below Groups A, B and C are as defined as follows:

(1) Group A: P2, Q3, R2, S3, T2, U2, V8, W6 (e.g., W6-1 and W6-2), X2, X3, Y2, Z2, AA2, AA3, AB2, AC12, AD7, AE4, AG2, AH7, AI2, AJ12, 201-214, 216-266, 268-424, 437-465, and 468-533;

(2) Group B: P2, Q3, R2, S3, T2, U2, V8, W6 (e.g., W6-1 and W6-2), X2, X3, Y2, Z2, AA2, AA3, AB2, AC12, AD7, AE4, AG2, 201-214, 216-266, 268-420; and

(3) Group C: AH7, 421-424, and 437-446.

Another embodiment of this invention is directed to compounds of Group A.

Another embodiment of this invention is directed to compounds of Group B.

Another embodiment of this invention is directed to compounds of Group C.

Another embodiment of this invention is directed to a pharmaceutically acceptable salt of a compound selected from the group consisting of the compounds of Group A. And in another example the salt is a salt of a compound selected from the group consisting of Group B. And in another example the salt is a salt of a compound selected from the group consisting of Group C.

Another embodiment of this invention is directed to a pharmaceutically acceptable ester of a compound selected from the group consisting of the compounds of Group A. And in another example the ester is an ester of a compound selected from the group consisting of Group B. And in another example the ester is an ester of a compound selected from the group consisting of Group C.

Another embodiment of this invention is directed to a solvate of a compound selected from the group consisting of the compounds of Group A. And in another example the solvate is a solvate of a compound selected from the group consisting of Group B. And in another example the solvate is a solvate of a compound selected from the group consisting of Group C.

Another embodiment of this invention is directed to a compound, selected from the group consisting of the compounds of Group A, in pure and isolated form. And in one example the compound is selected from the group consisting of the compounds in Group B. And in another example the compound is selected from the group consisting of the compounds in Group C.

Another embodiment of this invention is directed to a compound, selected from the group consisting of the compounds of Group A, in pure form. And in one example the compound is selected from the group consisting of the compounds in Group B. And in another example the compound is selected from the group consisting of the compounds in Group C.

Another embodiment of this invention is directed to a compound, selected from the group consisting of the compounds of Group A, in isolated form. And in one example the compound is selected from the group consisting of the compounds in Group B. And in another example the compound is selected from the group consisting of the compounds in Group C.

Another embodiment of this invention is directed to a pharmaceutical composition comprising a therapeutically effective amount of at least one compound selected from the group consisting of the compounds of Group A, or a pharmaceutically acceptable salt, solvate, or ester thereof, and at least one pharmaceutically acceptable carrier.

Another embodiment of this invention is directed to a pharmaceutical composition comprising an effective amount of one or more (e.g., one) compounds selected from the group consisting of the compounds of Group A, and a pharmaceutically acceptable carrier.

Another embodiment of this invention is directed to a pharmaceutical composition comprising an effective amount of a pharmaceutically acceptable salt of one or more (e.g., one) compounds selected from the group consisting of the compounds of Group A, and a pharmaceutically acceptable carrier.

Another embodiment of this invention is directed to a pharmaceutical composition comprising an effective amount of a pharmaceutically acceptable ester of one or more (e.g., one) compounds selected from the group consisting of the compounds of Group A, and a pharmaceutically acceptable carrier.

Another embodiment of this invention is directed to a pharmaceutical composition comprising an effective amount of a solvate of one or more (e.g., one) compounds selected from the group consisting of the compounds of Group A, and a pharmaceutically acceptable carrier.

Another embodiment of this invention is directed to a pharmaceutical composition comprising an effective amount of one or more (e.g., one) compounds selected from the group consisting of the compounds of Group A, and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising a therapeutically effective amount of at least one compound selected from the group consisting of the compounds of Group A, or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Another embodiment of this invention is directed to a pharmaceutical composition comprising an effective amount of one or more (e.g., one) compounds selected from the group consisting of the compounds of Group A, and effective amount of one or more BACE inhibitors, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more muscarinic antagonists (e.g., m₁ or m₂ antagonists), and a pharmaceutically acceptable carrier.

Another embodiment of this invention is directed to a pharmaceutical composition comprising an effective amount of one or more (e.g., one) compounds selected from the group consisting of the compounds of Group A, and effective amount of Exelon (rivastigmine), and a pharmaceutically acceptable carrier.

Another embodiment of this invention is directed to a pharmaceutical composition comprising an effective amount of one or more (e.g., one) compounds selected from the group consisting of the compounds of Group A, and effective amount of Cognex (tacrine), and a pharmaceutically acceptable carrier.

Another embodiment of this invention is directed to a pharmaceutical composition comprising an effective amount of one or more (e.g., one) compounds selected from the group consisting of the compounds of Group A, and effective amount of a Tau kinase inhibitor, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds selected from the group consisting of the compounds of Group A, and effective amount of one anti-Abeta vaccine (active immunization), and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more APP ligands, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more fibrates (for example, clofibrate, Clofibride, Etofibrate, Aluminium Clofibrate), and a pharmaceutically acceptable carrier

Another embodiment of this invention is directed to a pharmaceutical composition comprising an effective amount of one or more (e.g., one) compounds selected from the group consisting of the compounds of Group A, and effective amount of one or more LXR agonists, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more LRP mimics, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more 5-HT6 receptor antagonists, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more nicotinic receptor agonists, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more H3 receptor antagonists, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more histone deacetylase inhibitors, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more hsp90 inhibitors, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more m1 muscarinic receptor agonists, and a pharmaceutically acceptable carrier.

Another embodiment of this invention is directed to combinations, i.e., a pharmaceutical composition, comprising a pharmaceutically acceptable carrier, an effective (i.e., therapeutically effective) amount of one or more compounds selected from the group consisting of the compounds of Group A, in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1 H-inden-1-one hydrochloride, i.e., donepezil hydrochloride, available as the Aricept® brand of donepezil hydrochloride), Aβ antibody inhibitors, gamma secretase inhibitors and beta secretase inhibitors.

Another embodiment of this invention is directed to a pharmaceutical composition comprising an effective amount of one or more (e.g., one) compounds selected from the group consisting of the compounds of Group A, and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more one mGluR2/3 antagonists, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more PAI-1 inhibitors, and a pharmaceutically acceptable carrier.

Another embodiment of this invention is directed to a pharmaceutical composition comprising an effective amount of one or more (e.g., one) compounds selected from the group consisting of the compounds of Group A, and effective amount of one or more agents that can induce Abeta efflux such as gelsolin, and a pharmaceutically acceptable carrier.

Other embodiments of this invention are directed to any one of the above embodiments directed to pharmaceutical compositions wherein the compound is selected from the group consisting of the compounds in Group B.

Other embodiments of this invention are directed to any one of the above embodiments directed to pharmaceutical compositions wherein the compound is selected from the group consisting of the compounds in Group C.

The compounds selected from the group consisting of the compounds of Group A can be useful as gamma secretase modulators and can be useful in the treatment and prevention of diseases such as, for example, central nervous system disorders (such as Alzheimers disease and Downs Syndrome), mild cognitive impairment, glaucoma, cerebral amyloid angiopathy, stroke, dementia, microgliosis, brain inflammation, and olfactory function loss.

Another embodiment of this invention is directed to a method of treating a central nervous system disorder comprising administering a therapeutically effective amount of at least one compound, selected from the group consisting of the compounds of Group A, to a patient in need of such treatment.

Another embodiment of this invention is directed to a method of treating a central nervous system disorder comprising administering a therapeutically effective amount of a pharmaceutical composition comprising a therapeutically effective amount of at least one compound selected from the group consisting of the compounds of Group A, or a pharmaceutically acceptable salt, solvate, or ester thereof, and at least one pharmaceutically acceptable carrier.

Another embodiment of this invention is directed to a method of treating a central nervous system disorder comprising administering a therapeutically effective amount of a pharmaceutical composition comprising a therapeutically effective amount of at least one compound selected from the group consisting of the compounds of Group A, or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Another embodiment of this invention is directed to a method for modulating (including inhibiting, antagonizing and the like) gamma-secretase comprising administering an effective amount of one or more (e.g., one) compounds, selected from the group consisting of the compounds of Group A, to a patient in need of such treatment.

Another embodiment of this invention is directed to a method for modulating (including inhibiting, antagonizing and the like) gamma-secretase, comprising administering an effective amount of a compound, selected from the group consisting of the compounds of Group A, to a patient in need of treatment.

Another embodiment of this invention is directed to a method of treating one or more neurodegenerative diseases, comprising administering an effective amount of one or more (e.g., one) compounds, selected from the group consisting of the compounds of Group A, to a patient in need of treatment.

Another embodiment of this invention is directed to a method of treating one or more neurodegenerative diseases, comprising administering an effective amount of a compound, selected from the group consisting of the compounds of Group A, to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of one or more (e.g., one) compounds, selected from the group consisting of the compounds of Group A, to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of a compound, selected from the group consisting of the compounds of Group A, to a patient in need of treatment.

Another embodiment of this invention is directed to a method of treating Alzheimer's disease, comprising administering an effective amount of one or more (e.g., one) compounds, selected from the group consisting of the compounds of Group A, to a patient in need of treatment.

Another embodiment of this invention is directed to a method of treating Alzheimer's disease, comprising administering an effective amount of a compound, selected from the group consisting of the compounds of Group A, to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds, selected from the group consisting of the compounds of Group A, to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound, selected from the group consisting of the compounds of Group A, to a patient in need of treatment.

Another embodiment of this invention is directed to a method of treating mild cognitive impairment, comprising administering an effective amount of one or more (e.g., one) compounds, selected from the group consisting of the compounds of Group A, to a patient in need of treatment.

Another embodiment of this invention is directed to a method of treating glaucoma, comprising administering an effective amount of one or more (e.g., one) compounds, selected from the group consisting of the compounds of Group A, to a patient in need of treatment.

Another embodiment of this invention is directed to a method of treating cerebral amyloid angiopathy, comprising administering an effective amount of one or more (e.g., one) compounds, selected from the group consisting of the compounds of Group A, to a patient in need of treatment.

Another embodiment of this invention is directed to a method of treating stroke, comprising administering an effective amount of one or more (e.g., one) compounds, selected from the group consisting of the compounds of Group A, to a patient in need of treatment.

Another embodiment of this invention is directed to a method of treating dementia, comprising administering an effective amount of one or more (e.g., one) compounds, selected from the group consisting of the compounds of Group A, to a patient in need of treatment.

Another embodiment of this invention is directed to a method of treating microgliosis, comprising administering an effective amount of one or more (e.g., one) compounds, selected from the group consisting of the compounds of Group A, to a patient in need of treatment.

Another embodiment of this invention is directed to a method of treating brain inflammation, comprising administering an effective amount of one or more (e.g., one) compounds, selected from the group consisting of the compounds of Group A, to a patient in need of treatment.

Another embodiment of this invention is directed to a method of treating olfactory function loss, comprising administering an effective amount of one or more (e.g., one) compounds, selected from the group consisting of the compounds of Group A, to a patient in need of treatment.

Another embodiment of this invention is directed to a method of treating Downs syndrome, comprising administering an effective amount of one or more (e.g., one) compounds, selected from the group consisting of the compounds of Group A, to a patient in need of treatment.

Another embodiment of this invention is directed to a method of treating Downs syndrome, comprising administering an effective amount of a compound, selected from the group consisting of the compounds of Group A, to a patient in need of treatment.

Other embodiments of this invention are directed to any one of the above embodiments directed to methods of treating wherein the compound is selected from the group consisting of the compounds of Group B.

Other embodiments of this invention are directed to any one of the above embodiments directed to methods of treating wherein the compound is selected from the group consisting of Group C.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds, selected from the group consisting of the compounds of Group A, to a patient in need of treatment and the administration of an effective amount of one or more (e.g., one) other pharmaceutical active ingredients (e.g., drugs). The compounds selected from the group consisting of the compounds of Group A, and the other drugs, can be administered separately (i.e., each is in its own separate dosage form), or the compounds selected from the group consisting of the compounds of Group A can be combined with the other drugs in the same dosage form.

Thus, other embodiments of this invention are directed to any one of the methods of treatment, or methods of inhibiting, described herein, wherein an effective amount of a compound, selected from the group consisting of the compounds of Group A, is used in combination with an effective amount of one or more other pharmaceutically active ingredients (e.g., drugs). The other pharmaceutically active ingredients (i.e., drugs) are selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Another embodiment of this invention is directed to a method of treating Alzheimer's disease, comprising administering an effective amount of one or more (e.g., one) compounds, selected from the group consisting of the compounds of Group A, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1 H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of a compound, selected from the group consisting of the compounds of Group A, in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1 H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of one or more (e.g., one) compounds, selected from the group consisting of the compounds of Group A, in combination with an effective amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective amount of one or more (e.g., one) compounds, selected from the group consisting of the compounds of Group A, in combination with an effective amount of one or more BACE inhibitors.

Another embodiment of this invention is directed to a method of treating Alzheimer's disease, comprising administering an effective amount of one or more compounds, selected from the group consisting of the compounds of Group A, in combination with an effective amount of Exelon (rivastigmine).

Another embodiment of this invention is directed to a method of treating Alzheimer's disease, comprising administering an effective amount of one or more compounds, selected from the group consisting of the compounds of Group A, in combination with an effective amount of Cognex (tacrine).

Another embodiment of this invention is directed to a method of treating Alzheimer's disease, comprising administering an effective amount of one or more compounds, selected from the group consisting of the compounds of Group A, in combination with an effective amount of a Tau kinase inhibitor.

Another embodiment of this invention is directed to a method of treating Alzheimer's disease, comprising administering an effective amount of one or more compounds of Group A, in combination with an effective amount of one or more Tau kinase inhibitor (e.g., GSK3beta inhibitor, cdk5 inhibitor, ERK inhibitor).

This invention also provides a method of treating Alzheimer's disease, comprising administering an effective amount of one or more compounds, selected from the group consisting of the compounds of Group A, in combination with an effective amount of one anti-Abeta vaccination (active immunization).

Another embodiment of this invention is directed to a method of treating Alzheimer's disease, comprising administering an effective amount of one or more compounds, selected from the group consisting of the compounds of Group A, in combination with an effective amount of one or more APP ligands.

Another embodiment of this invention is directed to a method of treating Alzheimer's disease, comprising administering an effective amount of one or more compounds, selected from the group consisting of the compounds of Group A, in combination with an effective amount of one or more agents that upregulate insulin degrading enzyme and/or neprilysin.

Another embodiment of this invention is directed to a method of treating Alzheimer's disease, comprising administering an effective amount of one or more compounds, selected from the group consisting of the compounds of Group A, in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

This invention also provides a method of treating Alzheimer's disease, comprising administering an effective amount of one or more compounds, selected from the group consisting of the compounds of Group A, in combination with an effective amount of one or more fibrates (for example, clofibrate, Clofibride, Etofibrate, Aluminium Clofibrate).

Another embodiment of this invention is directed to a method of treating Alzheimer's disease, comprising administering an effective amount of one or more compounds, selected from the group consisting of the compounds of Group A, in combination with an effective amount of one or more LXR agonists.

Another embodiment of this invention is directed to a method of treating Alzheimer's disease, comprising administering an effective amount of one or more compounds, selected from the group consisting of the compounds of Group A, in combination with an effective amount of one or more LRP mimics.

Another embodiment of this invention is directed to a method of treating Alzheimer's disease, comprising administering an effective amount of one or more compounds, selected from the group consisting of the compounds of Group A, in combination with an effective amount of one or more 5-HT6 receptor antagonists.

Another embodiment of this invention is directed to a method of treating Alzheimer's disease, comprising administering an effective amount of one or more compounds selected from the group consisting of the compounds of Group A, in combination with an effective amount of one or more nicotinic receptor agonists.

Another embodiment of this invention is directed to a method of treating Alzheimer's disease, comprising administering an effective amount of one or more compounds, selected from the group consisting of the compounds of Group A, in combination with an effective amount of one or more H3 receptor antagonists.

This invention also provides a method of treating Alzheimer's disease, comprising administering an effective amount of one or more compounds, selected from the group consisting of the compounds of Group A, in combination with an effective amount of one or more histone deacetylase inhibitors.

Another embodiment of this invention is directed to a method of treating Alzheimer's disease, comprising administering an effective amount of one or more compounds, selected from the group consisting of the compounds of Group A, in combination with an effective amount of one or more hsp90 inhibitors.

Another embodiment of this invention is directed to a method of treating Alzheimer's disease, comprising administering an effective amount of one or more compounds, selected from the group consisting of the compounds of Group A, in combination with an effective amount of one or more m1 muscarinic receptor agonists.

Another embodiment of this invention is directed to a method of treating Alzheimer's disease, comprising administering an effective amount of one or more compounds, selected from the group consisting of the compounds of Group A, in combination with an effective amount of one or more 5-HT6 receptor antagonists mGluR1 or mGluR5 positive allosteric modulators or agonists

Another embodiment of this invention is directed to a method of treating Alzheimer's disease, comprising administering an effective amount of one or more compounds, selected from the group consisting of the compounds of Group A, in combination with an effective amount of one or more mGluR2/3 antagonists.

Another embodiment of this invention is directed to a method of treating Alzheimer's disease, comprising administering an effective amount of one or more compounds, selected from the group consisting of the compounds of Group A, in combination with an effective amount of one or more anti-inflammatory agents that can reduce neuroinflammation.

Another embodiment of this invention is directed to a method of treating Alzheimer's disease, comprising administering an effective amount of one or more compounds, selected from the group consisting of the compounds of Group A, in combination with an effective amount of one or more Prostaglandin EP2 receptor antagonists.

Another embodiment of this invention is directed to a method of treating Alzheimer's disease, comprising administering an effective amount of one or more compounds, selected from the group consisting of the compounds of Group A, in combination with an effective amount of one or more PAI-1 inhibitors.

Another embodiment of this invention is directed to a method of treating Alzheimer's disease, comprising administering an effective amount of one or more compounds, selected from the group consisting of the compounds of Group A, in combination with an effective amount of one or more agents that can induce Abeta efflux such as gelsolin.

Another embodiment of this invention is directed to a method of treating Downs syndrome, comprising administering an effective amount of one or more (e.g., one) compounds, selected from the group consisting of the compounds of Group A, in combination with an effective amount of one or more cholinesterase inhibitors (such as, for example, (±)-2,3-dihydro-5,6-dimethoxy-2-[[1-(phenylmethyl)-4-piperidinyl]methyl]-1 H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective amount of a compound, selected from the group consisting of the compounds of Group A, in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1 H-inden-1-one hydrochloride, i.e., donepezil hydrochloride, available as the Aricept® brand of donepezil hydrochloride), to a patient in need of treatment.

Other embodiments of this invention are directed to any one of the above embodiments directed to combination therapies (i.e., the above methods of treating wherein compounds selected from the group consisting of the compounds of Group A are used in combination with other pharmaceutically active ingredients, i.e., drugs) wherein the compound is selected from the group consisting of the compounds in Group B.

Other embodiments of this invention are directed to any one of the above embodiments directed to combination therapies (i.e., the above methods of treating wherein compounds selected from the group consisting of the compounds of Group A are used in combination with other pharmaceutically active ingredients, i.e., drugs) wherein the compound is selected from the group consisting of Group C.

This invention also provides a kit comprising, in separate containers, in a single package, pharmaceutical compositions for use in combination, wherein one container comprises an effective amount of a compound, selected from the group consisting of the compounds of Group A, in a pharmaceutically acceptable carrier, and another container (i.e., a second container) comprises an effective amount of another pharmaceutically active ingredient (as described above), the combined quantities of the compound, selected from the group consisting of the compounds of Group A, and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase, or (e) mild cognitive impairment, or (f) glaucoma, or (g) cerebral amyloid angiopathy, or (h) stroke, or (i) dementia, or (j) microgliosis, or (k) brain inflammation, or (l) olfactory function loss.

This invention also provides a kit comprising, in separate containers, in a single package, pharmaceutical compositions for use in combination, wherein one container comprises an effective amount of a compound, selected from the group consisting of the compounds of Group A, in a pharmaceutically acceptable carrier, and another container (i.e., a second container) comprises an effective amount of another pharmaceutically active ingredient (as described above), the combined quantities of the compound, selected from the group consisting of the compounds of Group A, and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Other embodiments of this invention are directed to any one of the above embodiments directed to kits wherein the compound is selected from the group consisting of the compounds in Group B.

Other embodiments of this invention are directed to any one of the above embodiments directed to kits wherein the compound is selected from the group consisting of the compounds in Group C.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ antagonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Fe. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. Application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated herein by reference thereto.

As used above, and throughout this disclosure, the following terms, unless otherwise indicated, shall be understood to have the following meanings:

“At least one” means one or more than one, for example, 1, 2 or 3, or in another example, 1 or 2, or in another example 1.

“One or more” with reference to the use of the compounds of this invention means that one or more than one compound is used, for example, 1, 2 or 3, or in another example, 1 or 2, or in another example 1.

“Patient” includes both human and animals.

“Mammal” means humans and other mammalian animals.

It is noted that the carbons in the compounds of Group A and other formulas herein may be replaced with 1 to 3 silicon atoms so long as all valency requirements are satisfied.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Group A or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Group A or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 Carbon atoms, 1-methyl-1-(alkanoyloxy)-ethyl having from 5 to 10 Carbon atoms, alkoxycarbonyloxymethyl having from 3 to 6 Carbon atoms, 1-(alkoxycarbonyloxy)ethyl having from 4 to 7 Carbon atoms, 1-methyl-1-(alkoxycarbonyloxy)ethyl having from 5 to 8 Carbon atoms, N-(alkoxycarbonyl)aminomethyl having from 3 to 9 Carbon atoms, 1-(N-(alkoxycarbonyl)amino)ethyl having from 4 to 10 Carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of Group A contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Group A incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93 (3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5 (1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” with reference to the amount of a compound of Group A, or another drug, used in a pharmaceutical composition, method of treatment or kit, means a therapeutically effective amount.

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Group A can form salts which are also within the scope of this invention. Reference to a compound of Group A herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Group A contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Group A may be formed, for example, by reacting a compound of Group A with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66 (1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Group A, and salts, solvates, esters and prodrugs thereof, may exist in their tautomeric form (for example, as an amide, enol, keto or imino ether). All such tautomeric forms are contemplated herein as part of the present invention.

The compounds of Group A may contain asymmetric or chiral centers, and, therefore, exist in different stereoisomeric forms. It is intended that all stereoisomeric forms of the compounds of Group A as well as mixtures thereof, including racemic mixtures, form part of the present invention. In addition, the present invention embraces all geometric and positional isomers. For example, if a compound of Group A incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Group A may be atropisomers (e.g., substituted biaryls) and are considered as part of this invention. Enantiomers can also be separated by use of chiral HPLC column.

It is also possible that the compounds of Group A may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Group A incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl and ¹²³I, respectively.

Certain isotopically-labelled compounds of the invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of the invention can be useful for medical imaging purposes. E.g., those labeled with positron-emitting isotopes like ¹¹C or ¹⁸F can be useful for application in Positron Emission Tomography (PET) and those labeled with gamma ray emitting isotopes like ¹²³I can be useful for application in Single photon emission computed tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Additionally, isotopic substitution at a site where epimerization occurs may slow or reduce the epimerization process and thereby retain the more active or efficacious form of the compound for a longer period of time. Isotopically labeled compounds of the invention, in particular those containing isotopes with longer half lives (T½>1 day), can generally be prepared by following procedures analogous to those disclosed in the Schemes and/or in the Examples herein below, by substituting an appropriate isotopically labeled reagent for a non-isotopically labeled reagent.

Polymorphic forms of the compounds of Group A, and of the salts, solvates, esters and prodrugs of the compounds of Group A, are intended to be included in the present invention.

The compounds according to the invention can have pharmacological properties; in particular, the compounds of Group A can be modulators of gamma secretase (including inhibitors, antagonists and the like).

More specifically, the compounds of Group A can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Group A, or a pharmaceutically acceptable salt, solvate, ester or prodrug of said compound to the mammal.

A preferred dosage is about 0.001 to 500 mg/kg of body weight/day of the compound of Group A. An especially preferred dosage is about 0.01 to 25 mg/kg of body weight/day of a compound of Group A, or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Group A, or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Group A, or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Group A, or a pharmaceutically acceptable salt, solvate, ester or prodrug of said compound and a pharmaceutically acceptable carrier, vehicle or diluent.

Yet another aspect of this invention is a kit comprising an amount of at least one compound of Group A, or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following preparations and examples which should not be construed to limit the scope of the disclosure. Alternative mechanistic pathways and analogous structures will be apparent to those skilled in the art. Reagents and reaction conditions can be changed according to the knowledge of those skilled in the art.

Where NMR data are presented, ¹H spectra were obtained on either a Varian VXR-200 (200 MHz, ¹H), Varian Gemini-300 (300 MHz) or XL-400 (400 MHz) and are reported as ppm down field from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min—10% CH₃CN, 5 min—95% CH₃CN, 7 min—95% CH₃CN, 7.5 min—10% CH₃CN, 9 min—stop. The observed parent ion is given.

The following solvents and reagents may be referred to by their abbreviations in parenthesis:
DCE means 1,2-dichloroethane
DCM: dichloromethane (CH₂Cl₂)
DEA means diethylamine
DEAD means diethyl azodicarboxylate
DIPEA means diisopropylethylamine
DMF means N,N-dimethylformamide
DMSO means dimethylsulfoxide.
EDCI means (3-(dimethylamino)propyl)ethyl carbodiimide hydrochloride
ethyl acetate: AcOEt or EtOAc
ethanol: EtOH
grams: g
high resolution mass spectrometry: HRMS
liquid chromatography mass spectrometry: LCMS
Me means methyl
methanol: MeOH
microliters: μl
milligrams: mg
milliliters: mL
millimoles: mmol
nuclear magnetic resonance spectroscopy: NMR
SM: Starting Material
TBAF means tetrabutyl ammonium fluoride
TBS means tert-butyldimethylsilyl
Thin layer chromatography: TLC
t-BU: tert-butyl
triethylamine: Et₃N or TEA
rt or r.t.: room temperature (ambient), about 25° C.

### EXAMPLES

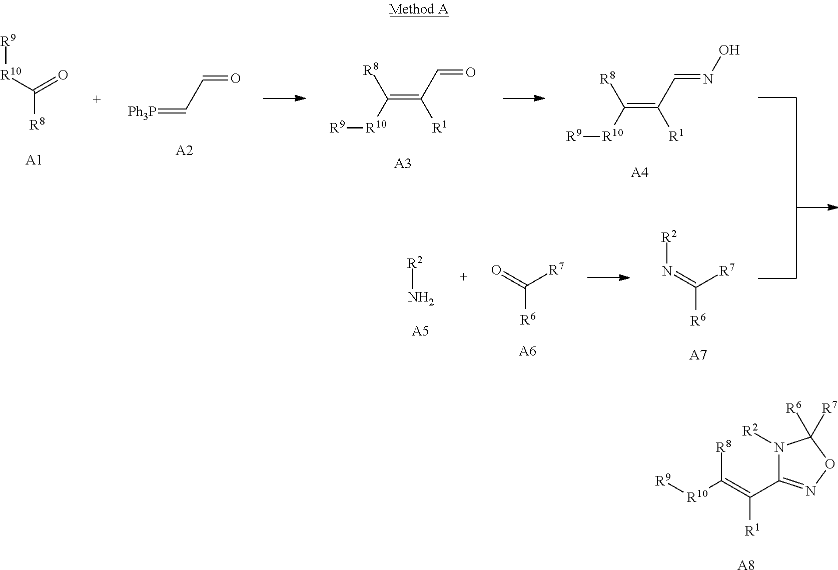

### Method A, Step 1

The following method was adapted for the oxadiazoline synthesis (Tsuge, Otohiko; Kanemasa, Shuji; Suga, Hiroyuki; Nakagawa, Norihiko. Bulletin of the Chemical Society of Japan (1987), 60 (7), 2463-73).

Compound A1 (R⁸═H, R¹⁰=3-MeO-Phenyl, R⁹=4-(4-Methylimidazol-1-yl), 3 g) and A2 (4.2 g) in 135 ml of anhydrous THF was heated at 100° C. in a sealed tube under nitrogen overnight. Solvent was evaporated and residue chromatographed using a silica gel column eluted with EtOAc/Hexane to give 2.7 g of A3 (R¹═H, R⁸═H, R¹⁰=3-OMePhenyl, R⁹=4-(4-Methyl-imidazol-1-yl)).

### Method A, Step 2

A3 (R¹═H, R⁸═H, R¹⁰=3-MeO-Phenyl, R⁹=4-(4-Methyl-imidazol-1-yl), 2.7 g) and potassium acetate (1.4 g) in 120 mL MeOH was cooled to 0° C. before hydroxylamine hydrochloride (1 g) was added. The reaction mixture was stirred for 90 min before the solvent was evaporated. The residue was partitioned in EtOAc and brine. The organic layer was dried over anhydrous Na₂SO₄. The crude was purified on C18 reverse phase column to give 1 g of A4 (R¹═H, R⁸═H, R¹⁰=3-MeO-Phenyl, R⁹=4-(4-Methyl-imidazol-1-yl)).

MS (M+1): 258.

### Method A, Step 3

A mixture of A5 (R²=3-MeO-propyl, 3 mL) and A6 (R⁶=Me, R⁷=p-F-phenyl, 1.2 mL) in a sealed tube was heated at 50° C. with 2 g of 4 Å molecular sieves under nitrogen for 3 h and r.t. for 72 h. The volatile was removed to give A7 (R²=3-MeO-Propyl, R⁶=Me, R⁷=p-F-Phenyl) as an oil which was used for next step without further purification.

### Method A, Step 4

A mixture of A4 (R¹═H, R⁸═H, R¹⁰=3-MeO-Phenyl, R⁹=4-(4-Methyl-imidazol-1-yl), 100 mg), N-Chlorosuccinimide (51.9 mg) and pyridine (8 uL) in 1.2 mL of DCM was stirred at r.t. for 10 min followed by addition of A7 (R²=3-OMePropyl, R⁶=Me, R⁷=p-F-Phenyl) and TEA (0.8 mL). The reaction mixture was stirred at r.t. overnight before it was diluted with DCM, washed with brine, dried over anhydrous sodium sulfate. The solvent was removed and residue purified via a reverse phase column eluted with MeCN/Water with 0.1% formic acid to give product A8 (R¹═H, R²=3-OMePropyl, R⁶=Me, R⁷=p-F-Phenyl, R⁸═H, R¹⁰=3-MeO-Phenyl, R⁹=4-(4-Methyl-imidazol-1-yl).

¹H NMR (CDCl3, ppm): 7.96 (br, 1 H), 7.59-7.55 (m, 2H), 7.48-7.44 (d, 1 H), 7.27-7.5 (m, 1 H), 7.18-7.16 (m, 1 H), 7.12 (m, 2H), 7.09-7.05 (t, 1 H), 6.96 (br, 1 H), 6.57-6.53 (d, 1 H), 3.89 (s, 3H), 3.27 (s, 3H), 3.29-3.14 (m, 4H), 2.32 (s, 3H), 1.89 (s, 3H), 1.55 (br, 2H). MS (ES-LCMS, M+1) 465.

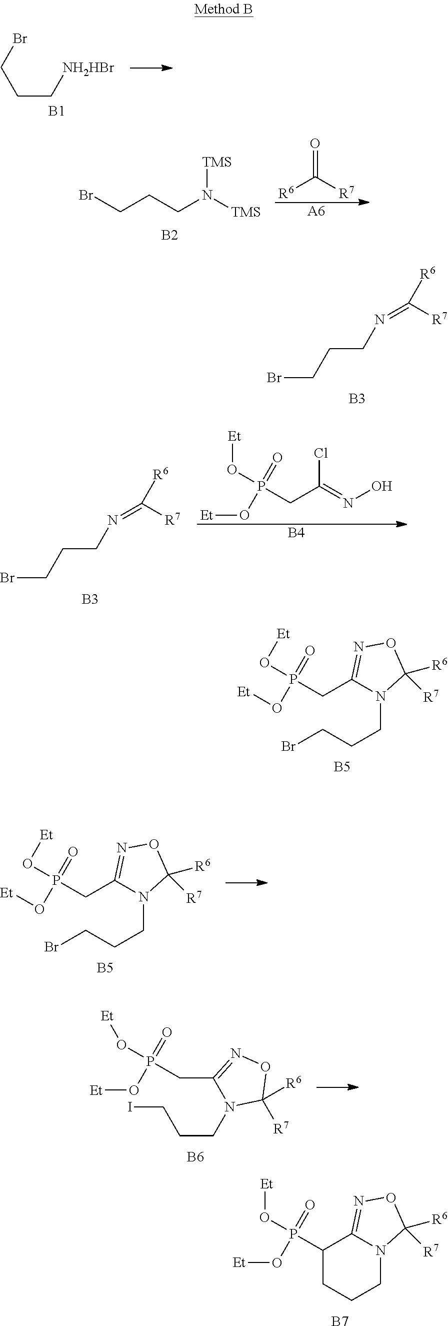

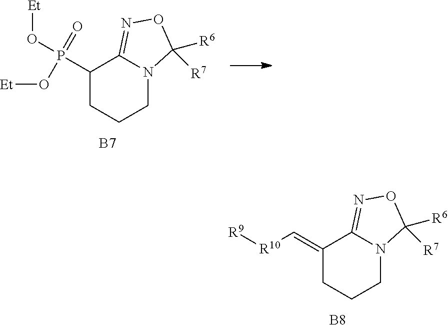

Method B, step 1:

Triethylamine (10.5 mL) was added slowly to a stirred suspension of B1 (5 g) in 66 mL of anhydrous DCM at 0° C. under nitrogen atmosphere. A solution of chlorotrimethylsilane (6.4 mL) in 12 mL in anhydrous DCM was added slowly to the above suspension. The reaction mixture was stirred at r.t. overnight before filtration to remove precipitate. The filtrate was evaporated and the residue oil was redissolved in 150 mL diethyl ether, stirred for 15 min, filtered and concentrated to give 5.7 g of B2.

### Method B, Step 2

A catalytic amount of trimethylsilyl trifluoromethanesulfonate was added to a stirred mixture of B2 (4.7 g) and A6 (3.3 g, R⁷=p-F-Phenyl and R⁶=carboethoxyl) in 33 mL of anhydrous DCM at r.t. under nitrogen atmosphere. The reaction mixture was refluxed for 48 h before cooled to r.t. and sequentially washed with cold NaHCO₃:water (1:1) and cold half-saturated brine. The organic phase was dried over anhydrous sodium sulfate, filtered and solvent removed to give 5 g of B3 (R⁷=p-F-Phenyl and R⁶=carboethoxyl).

### Method B, Step 3

A solution of B3 (R⁷=p-F-Phenyl and R⁶=carboethoxyl) (500 mg, 1 equiv.) in 2.3 mL of anhydrous DMF was slowly added to a solution of B4 (1.3 equiv. obtained following a reference procedure: Tsuge, Otohiko; Kanemasa, Shuji; Suga, Hiroyuki; Nakagawa, Norihiko Bulletin of the Chemical Society of Japan (1987), 60 (7), 2463-73) in 0.5 mL of anhydrous DMF at 0° C. under nitrogen atmosphere. A solution of TEA (0.33 mL, 1.5 equiv.) in 0.4 mL of anhydrous DMF was slowly added to the above reaction mixture. The reaction mixture was stirred at r.t. overnight before dilution with 20 mL of diethyl ether and 20 mL half-saturated brine. The aqueous phase was extracted with EtOAC:hexane (7:3). The organic phase was washed with half-saturated brine then, dried over anhydrous sodium sulfate. The solvent was evaporated and the residue was purified via a flash silica gel column eluted with DCM/EtOAc with 1% isopropanol to give B5 (198 mg, R⁷=p-F-Phenyl and R⁶=carboethoxyl).

¹H NMR (CDCl₃, ppm): δ7.45-7.42 (m, 2H), 7.07-7.03 (t, 2H), 4.27-4.24 (m, 2H), 4.17-4.10 (m, 4H), 3.52-3.40 (m, 1 H), 3.36-3.29 (m, 2H), 3.17-3.14 (m, 1 H), 2.95-2.89 (dd, 2H), 1.76-1.40 (m, 2H), 1.31-1.24 (m, 9H).

### Method B, Step 4

A mixture of B5 (6.2 g, R⁷=p-F-Phenyl and R⁶=carboethoxyl) and sodium iodide in 90 mL acetone was stirred overnight at reflux. The reaction mixture was diluted with 1 L diethyl ether and vigorously stirred for 30 min. The precipitate was filtered and the filtrate was washed with sodium thiosulfate (10.6 g) in brine and partitioned between diethyl ether and brine. The organic phase was dried over anhydrous magnesium sulfate, filtered and solvent evaporated. The residue was purified by a flash silica gel column and eluted with DCM/EtOAc to give 3 g of B6 (3 g, R⁷=p-F-Phenyl and R⁶=carboethoxyl)

### Method B, Step 5

A solution of t-BuOK (1.6 g) in 54 mL of anhydrous THF was added dropwise to a stirred solution of B6 (5.4 g, R⁷=p-F-Phenyl and R⁶=carboethoxyl) in 40 mL of anhydrous THF at −65° C. under nitrogen atmosphere. The reaction mixture was stirred between −65° C. and −40° C. until SM was consumed. The reaction mixture was quenched with iced brine, and extracted with EtOAc. The organic phase was washed with NH₄Cl and brine, dried over anhydrous magnesium sulfate, filtered and solvent evaporated. The residue was purified by a flash silica gel column and eluted with DCM/EtOAc to give 2.2 g of B7 (R⁷=p-F-Phenyl and R⁶=carboethoxyl).

¹H NMR (CDCl₃, ppm): δ7.47-7.41 (m, 2H), 7.24-7.06 (m, 2H), 4.34-4.09 (m, 6H), 3.42-3.30 (m, 1 H), 3.14-3.08 (m, 1 H), 2.78-2.59 (m, 1 H), 2.16-1.60 (m, 4H), 1.36-1.22 (m, 9H).

### Method B, Step 6

A solution of t-BuOK (733 mg) in 20.5 mL of anhydrous THF was added dropwise to a stirred mixture of B7 (R⁷=p-F-Phenyl and R⁶=carboethoxyl, 2.2 g) and A1 (1 g, R¹⁰=3-MeO-Phenyl, R⁹=4-(4-Methyl-imidazol-1-yl) and R⁸═H) in 29.5 mL of anhydrous THF at −70° C. under nitrogen atmosphere. The reaction mixture was stirred between −70° C. and −30° C. until starting material were consumed. The reaction was quenched with iced brine, and extracted with EtOAc. The organic phase was washed with aqueous NH₄Cl and brine, dried over anhydrous magnesium sulfate, filtered and solvent evaporated to give B8 (R⁷=p-F-Phenyl and R⁶=carboethoxyl, R¹⁰=3-MeO-Phenyl, R⁹=4-(4-Methyl-imidazol-1-yl)) after purification. Compound B8 (R⁷=p-F-Phenyl and R⁶=carboethoxyl, R¹⁰=3-MeO-Phenyl, R⁹=4-(4-Methyl-imidazol-1-yl)) was resolved by chiral AS column and eluted with Hexanes/Isopropanol with 0.1% DEA to give 930 mg of enantiomer A (B9) of B8 (R⁶=p-F-Phenyl, R⁷=carboethoxyl and R¹⁰=3-MeO-Phenyl, R⁹=4-(4-Methyl-imidazol-1-yl)) and 849 mg of enantiomer B (B10) of B8 (R⁷=p-F-Phenyl and R⁶=carboethoxyl, R¹⁰=3-MeO-Phenyl, R⁹=4-(4-Methyl-imidazol-1-yl)).

¹H NMR (CDCl₃, ppm) of the enantiomer A (B9) (R⁷=p-F-Phenyl and R⁶=carboethoxyl, R¹⁰=3-MeO-Phenyl, R⁶=4-(4-Methyl-imidazol-1-yl)): δ 7.68 (s, 1 H), 7.51-7.46 (m, 3H), 7.22-7.20 (d, 1 H), 7.11-7.07 (t, 2H), 6.98-6.90 (m, 3H), 4.36-4.28 (m, 2H), 3.81 (s, 3H), 3.58-3.52 (m, 1 H), 2.88-2.82 (m, 1 H), 2.74-2.65 (m, 2H), 2.26 (s, 3H), 1.97-1.95 (m, 1 H), 1.75-1.68 (m, 1 H), 1.33-1.29 (t, 3H). MS (ES-LCMS, M+1) 491.

Alternatively, B3 can be made by the following procedure:

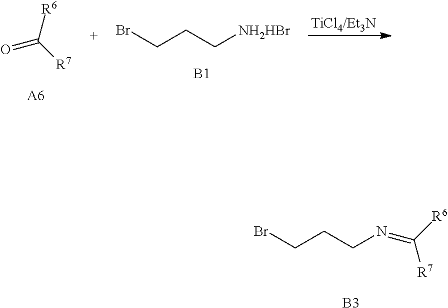

Triethylamine (40 mL, 8 eq) was added slowly to a solution of A6 (R⁶=Me and R⁷=p-F-phenyl; 5 g, 1 eq) and B1 (10.3 g, 1.3 eq) in 35 mL anh. DMF and 10 mL DCM while vigorously stirring under nitrogen. A solution of TiCl₄ (3.6 mL, 0.9 eq) in 29 mL DCM was added dropwise at 0° C. The reaction suspension was vigorously stirred at rt. overnight. The reaction mixture was mixed with ether, filtered and the filtrate was washed with ice cold brine 4 times and dried over anhydrous Na₂SO₄ to give 6.8 g of B3 (R⁶=Me and R⁷=p-F-phenyl).

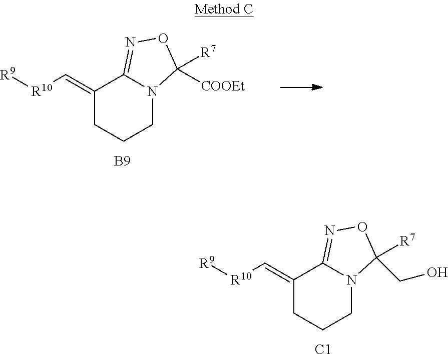

Solid sodium borohydride (57.3 mg) was added to a stirred solution of B9 (400 mg; R⁷=p-F-Phenyl and R⁶=carboethoxyl, R¹⁰=3-MeO-Phenyl, R⁶=4-(4-Methyl-imidazol-1-yl)) in 9 mL of MeOH:EtOH (1:2) at 0° C. under nitrogen atmosphere. The reaction mixture was stirred at 0° C. for 1 h and then at r.t. for 1 hr, quenched with iced brine, and extracted with EtOAc. The organic phase was dried over anhydrous sodium sulfate and evaporated. Residue was purified via a reverse-phase column with MeCN/Water with 0.1% formic acid to give C1 (R⁷=p-F-Phenyl, R¹⁰=3-MeO-Phenyl, R⁹=4-(4-Methyl-imidazol-1-yl))

¹H NMR (CDCl₃, ppm) of the enantiomer A of C1 (R⁶=p-F-Phenyl, R¹⁰=3-MeO-Phenyl, R⁹=4-(4-Methyl-imidazol-1-yl)): δ 7.22 (s, 1 H), 7.50-7.44 (m, 3H), 7.24-7.22 (d, 1 H), 7.11-7.07 (t, 2H), 6.98-6.91 (m, 3H), 4.21-4.18 (dd, 2H), 3.85 (s, 3H), 3.36-3.30 (m, 1 H), 3.00-2.93 (m, 1 H), 2.71-2.70 (m, 2H), 2.28 (s, 3H), 1.99-1.82 (m, 2H). MS (ES-LCMS, M+1) 449.

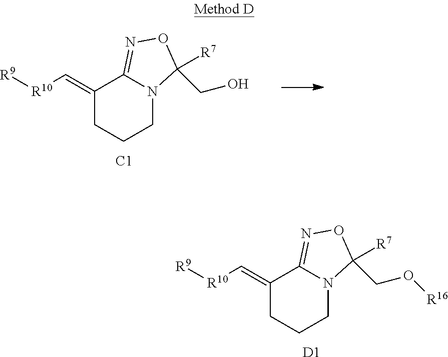

Sodium hydride (2 mg, 60% in mineral oil) and R¹⁶—I (16.6 mg, R¹⁶=Me) was added to a stirred solution of the racemate of C1 (7.5 mg, R⁷=p-F-Phenyl, R¹⁰=3-MeO-Phenyl, R⁹=4-(4-Methyl-imidazol-1-yl)) in 0.2 mL of anhydrous DMF at r.t. under nitrogen atmosphere. The reaction mixture was stirred at r.t. for 15 min, quenched with iced brine, and extracted with EtOAc. The organic phase was dried over anhydrous sodium sulfate and evaporated. Residue was purified via a reverse-phase column with MeCN/Water with 0.1% formic acid to give the racemate of D1 (R⁷=p-F-Phenyl, R¹⁰=3-MeO-Phenyl, R⁹=4-(4-Methyl-imidazol-1-yl) and R¹⁶=Me).

¹H NMR (CDCl₃, ppm) of the racemate of D1 (R⁷=p-F-Phenyl, R¹⁰=3-MeO-Phenyl, R⁹=4-(4-Methyl-imidazol-1-yl), R¹⁶=Me): δ 7.97 (s, 1 H), 7.55-7.50 (m, 2H), 7.47 (s, 1 H), 7.24-7.19 (d, 1 H), 7.10-7.01 (t, 2H), 6.99-6.92 (m, 3H), 4.08-3.88 (dd, 2H), 3.84 (s, 3H), 3.50 (s, 3H), 3.31-3.25 (m, 1 H), 2.98-2.93 (m, 1 H), 2.80-2.50 (m, 2H), 2.32 (s, 3H), 1.93-1.78 (m, 2H). MS (ES-LCMS, M+1) 463.

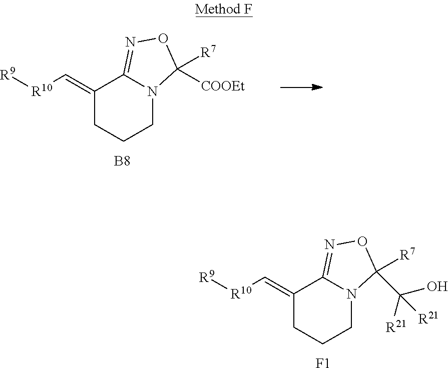

A solution of methyl magnesium bromide (0.14 mL, 3 M in ether) was added dropwise to a stirred solution the enantiomer A of B8 (R⁷=p-F-Phenyl, R¹⁰=3-MeO-Phenyl, R⁹=4-(4-Methyl-imidazol-1-yl), 50 mg) in 1 mL of THF at −50° C. under nitrogen atmosphere. The reaction mixture was stirred between −50° C. and 10° C. until the SM was consumed. The reaction was quenched with iced aqueous NH₄Cl, stirred for 30 min, and then extracted with EtOAc. The organic phase was dried over anhydrous magnesium sulfate, filtered and solvent evaporated. The residue was purified via a reverse-phase column with MeCN/Water with 0.1% formic acid to give the enantiomer A of F1 (R⁷=p-F-Phenyl, R¹⁰=3-MeO-Phenyl, R⁹=4-(4-Methyl-imidazol-1-yl) and R²¹═R²¹=Me).

¹H NMR (CDCl₃, ppm) of the enantiomer A of F1 (R⁷=p-F-Phenyl, R¹⁰=3-MeO-Phenyl, R⁹=4-(4-Methyl-imidazol-1-yl) and R²¹═R²¹=Me): δ 7.86-7.82 (m, 2H), 7.72 (br, 1 H), 7.42 (s, 1 H), 7.23-7.20 (d, 1 H), 7.10-7.04 (t, 2H), 6.98-6.93 (m, 3H), 3.81 (s, 3H), 3.75-3.70 (m, 1 H), 3.08-3.02 (m, 1 H), 2.85-2.81 (m, 1 H), 2.42-2.33 (m, 1 H), 2.28 (s, 3H), 1.88-1.80 (m, 2H), 1.48 (s, 3H), 1.18 (s, 3H). MS (ES-LCMS, M+1) 477.

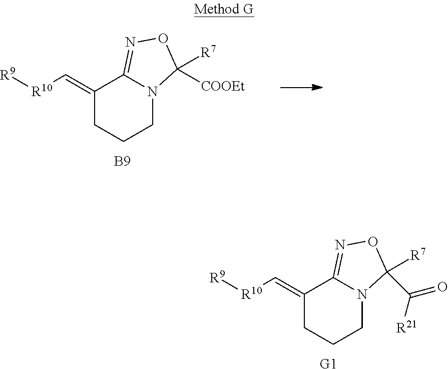

A solution of methyl magnesium bromide (0.3 mL, 3 M in ether) and TEA (0.4 mL) in 0.7 mL of THF was added dropwise to a stirred solution of the enantiomer A (B9) of B8 (250 mg, R⁷=p-F-Phenyl, R¹⁰=3-MeO-Phenyl, R⁹=4-(4-Methyl-imidazol-1-yl)) in 1.5 mL of THF at −50° C. under nitrogen atmosphere. The reaction mixture was stirred between −50° C. and 15° C. until SM was consumed, quenched with iced aqueous NH₄Cl, stirred for 30 min, and then extracted with EtOAc. The organic phase was dried over anhydrous magnesium sulfate and evaporated. Residue was purified via a reversed-phase column with MeCN/Water with 0.1% formic acid to give 80 mg of G1 (R⁷=p-F-Phenyl, R¹⁰=3-MeO-Phenyl, R⁹=4-(4-Methyl-imidazol-1-yl) and R²¹=Me).

¹H NMR (CDCl₃, ppm) of the enantiomer A of G1 (R⁷=p-F-Phenyl, R¹⁰=3-MeO-Phenyl, R⁹=4-(4-Methyl-imidazol-1-yl) and R²¹=Me): δ 7.07 (s, 1 H), 7.47-7.34 (m, 3H), 7.21-7.19 (d, 1 H), 7.14-7.08 (t, 2H), 6.96-6.88 (m, 3H), 3.80 (s, 3H), 3.58-3.53 (m, 1 H), 2.81-2.77 (m, 1 H), 2.71-2.58 (m, 2H), 2.25 (m, 6H), 1.92-1.85 (m, 1 H), 1.72-1.65 (m, 1 H). MS (ES-LCMS, M+1) 461.

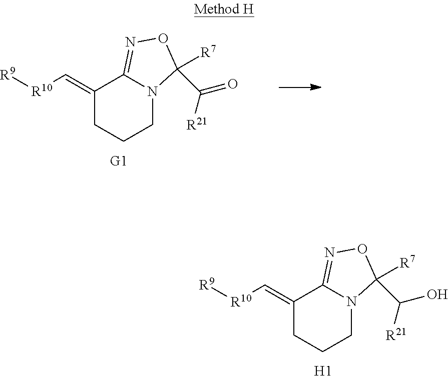

A suspension of sodium borohydride (6.6 mg) in 0.5 mL of EtOH was added slowly to a stirred solution of G1 (R⁷=p-F-Phenyl, R¹⁰=3-MeO-Phenyl, R⁹=4-(4-Methyl-imidazol-1-yl) and R²¹=Me; 80 mg) in 4.8 mL of MeOH:EtOH (1:2) at 0° C. The reaction mixture was stirred at 0° C. for 1 h before quenched with iced brine and extracted with EtOAc. The organic phase was dried over anhydrous magnesium sulfate, filtered and solvent evaporated. Residue was purified via a reverse-phase column with MeCN/Water with 0.1% formic acid to give 32 mg of diastereomer 1 of product H1 (R⁷=p-F-Phenyl, R¹⁰=3-MeO-Phenyl, R⁹=4-(4-Methyl-imidazol-1-yl) and R²¹=Me) and 24.6 mg of diastereomer 2 of product H1 (R⁷=p-F-Phenyl, R¹⁰=3-MeO-Phenyl, R⁹=4-(4-Methyl-imidazol-1-yl) and R²¹=Me).

¹H NMR (CDCl₃, ppm) of diastereomer 1 of product H1 (R⁷=p-F-Phenyl, R¹⁰=3-MeO-Phenyl, R⁹=4-(4-Methyl-imidazol-1-yl) and R²¹=Me): δ 8.03 (s, 1 H), 7.63-7.59 (m, 2H), 7.44 (s, 1 H), 7.24-7.22 (d, 1 H), 7.10-7.06 (t, 2H), 6.98-6.92 (m, 3H), 4.58-4.53 (m, 1 H), 3.83 (s, 3H), 3.27-3.22 (m, 1 H), 2.88-2.83 (m, 1 H), 2.75-2.71 (m, 1 H), 2.54-2.48 (m, 1 H), 2.31 (s, 3H), 1.8-1.80 (m, 2H), 1.37-1.36 (d, 3H). MS (ES-LCMS, M+1) 463.

¹H NMR (CDCl₃, ppm) of diastereomer 2 of product H1 (R⁷=p-F-Phenyl, R¹⁰=3-MeO-Phenyl, R⁹=4-(4-Methyl-imidazol-1-yl), and R²¹=Me): δ 7.94 (s, 1 H), 7.50-7.42 (m, 2H), 7.41 (s, 1 H), 7.23-7.21 (d, 1 H), 7.11-7.07 (t, 2H), 6.98-6.91 (m, 3H), 4.53-4.48 (m, 1 H), 3.82 (s, 3H), 3.42-3.37 (m, 1 H), 2.99-2.94 (m, 1 H), 2.77-2.71 (m, 1 H), 2.53-2.48 (m, 1 H), 2.30 (s, 3H), 1.99-1.80 (m, 2H), 1.15-1.13 (d, 3H). MS (ES-LCMS, M+1) 463.

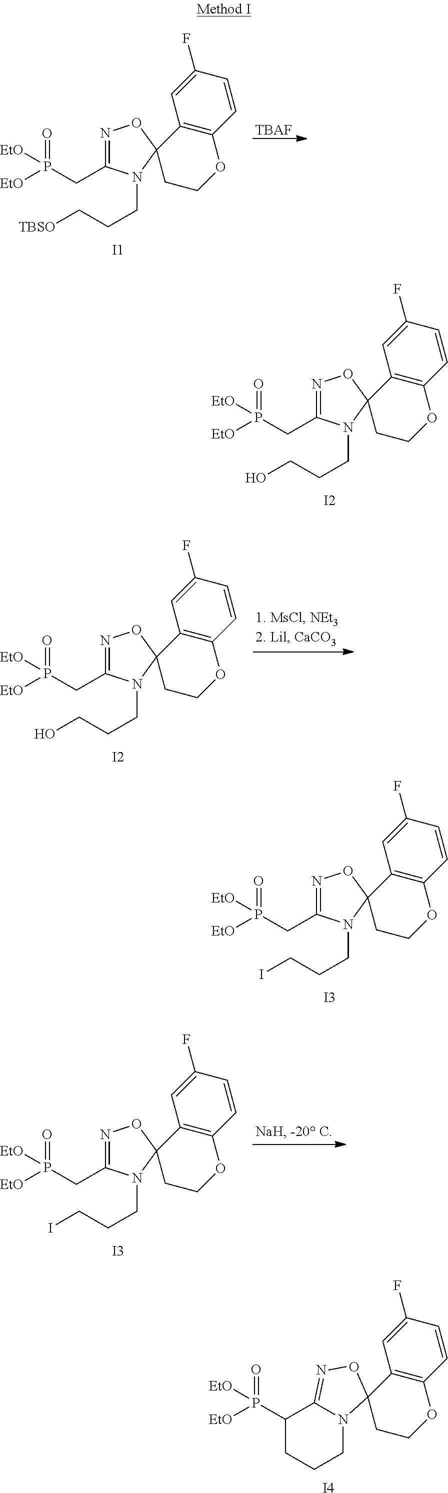

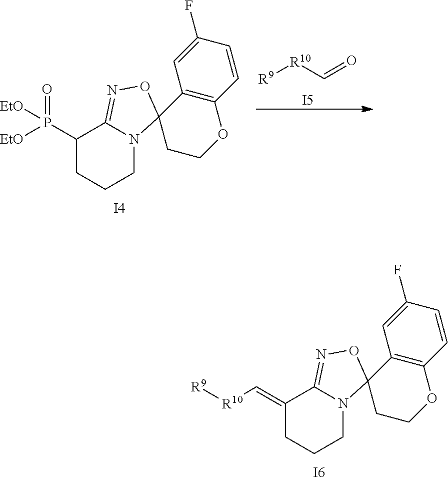

### Method I, Step 1

Compound II (prepared using method similar to B1 to B5, 0.90 g, 1.7 mmole) was dissolved in 50 ml THF and tetrabutylammonium fluoride(1 M in THF, 3.4 ml) was added. The reaction was stirred at room temperature for 1 h before 100 ml EtOAc and 100 ml brine were added. The organic layer was washed with brine (2×100 ml), dried with Na₂SO₄, filtered and solvent evaporated. The residue was purified by column (EtOAc/MeOH from 100/0 to 90/10 in 45 minutes, 80 g silica) to give 12. Yield, 0.49 g, 69%. ¹H NMR (CDCl₃, ppm): δ 7.23 (dd, 1 H), 6.96 (m, 1 H), 6.80 (dd, 1 H), 4.14-4.40 (m, 6H), 3.60 (m, 1 H), 3.48 (m, 1 H), 3.38 (m, 2H), 2.94-3.15 (m, 4H), 2.40 (m, 1 H), 2.21 (m, 1 H), 1.40 (m, 6H).

### Method I, Step 2

Compound 12 (0.49 g, 1.18 mmole) was dissolved in 50 ml DCM followed by addition of Mesyl chloride (0.2 g) and triethylamine (0.18 g). The reaction was stirred at room temperature for 10 minutes before 50 ml DCM was added and the organic layer was washed with brine (2×100 ml), dried with Na₂SO₄, filtered and solvent evaporated. The residue was dissolved in 50 ml AcCN followed by addition of Lil (0.31 g) and CaCO₃ (0.24 g). The reaction was stirred at 80° C. for 1 hour before 70 ml EtOAc was added and the organic layer was washed with brine (2×100 ml), dried with Na₂SO₄, filtered and solvent evaporated. The residue was purified by column (EtOAc/hexane from 50/50 to 100/0 in 35 minutes, 12+40 g silica) to give compound 13. Yield: 0.4 g, 64%. ¹H NMR (CDCl₃, ppm): δ 7.23 (dd, 1 H), 6.96 (m, 1 H), 6.80 (dd, 1 H), 4.18-4.38 (m, 6H), 3.38 (m, 1 H), 3.19 (m, 1 H), 2.90-3.10 (m, 4H), 2.34 (m, 1 H), 2.22 (m, 1 H), 1.87 (m, 2H), 1.39 (m, 6H).

### Method I, Step 3

Compound I3 (0.4 g, 0.78 mmole) was dissolved in 50 ml THF and the reaction was cooled to −78° C. before Sodium hydride (60% in oil, 62 mg) was added and the reaction was slowly warmed up to −20° C. was then stirred at −20° C. for 2 hours followed by addition of 100 ml water and 100 ml EtOAc. The organic layer was washed with brine (2×100 ml), dried with Na₂SO₄ and concentrated. The residue was purified by column (EtOAc/MeOH from 100/0 to 90/10 in 25 minutes) to give I3 as a mixture of two steroismers. Total yield: 0.2 g, 64%. ¹H NMR (CDCl₃, ppm) of I4: 7.32 (dd, 0.7H), 7.10 (dd, 0.3H), 6.96 (m, 1 H), 6.80 (m, 1 H), 4.15-4.40 (m, 6H), 3.07-3.25 (m, 1 H), 2.80-3.00 (m, 2H), 1.79-2.50 (m, 6H), 1.38 (m, 6H).

### Method I, Step 4

Compound 14 (193 mg, 0.51 mmole) was dissolved in THF and the reaction was cooled to −78° C. Butyllithium (2.5 ml in hexane, 0.22 ml) was added and the reaction was stirred at −78° C. for 30 minutes before compound 15 (R¹⁰=3-MeO-Phenyl, R⁹=4-(4-Methylimidazol-1-yl)) (110 mg, 0.51 mmole) in 10 ml THF (Pre-cooled to −78° C.) was added. The reaction was stirred at −78° C. for 1 hour, then at room temperature for one hour before solvent was removed and the residue partitioned between 100 ml EtOAc and 100 ml water. The organic layer was washed with water (2×100 ml), dried with Na₂SO₄ and concentrated. The residue was dissolved in 30 ml THF was treated with 50 mg NaBH₄ to reduce excess aldehyde to alcohol. The product was purified by column (DCM/MeOH from 100/0 to 90/10 in 25 minutes) to give compound 16 (R¹⁰=3-MeO-Phenyl, R⁹=4-(4-Methylimidazol-1-yl)) as an 86:14/E:Z mixture. Yield: 102 mg, 44%. The pure E isomer was obtained using chiral AS column separation. ¹H NMR (CDCl₃, ppm): δ 7.76 (s, 1 H), 7.55 (s, 1 H), 7.27 (d, 1 H), 722 (dd, 1 H), 6.93-7.06 (m, 4H), 6.84 (dd, 1 H), 4.37 (m, 2H), 3.87 (s, 3H), 2.90-3.10 (m, 3H), 2.58 (m, 1 H), 2.26-2.32 (m, 5H), 1.98 (m, 1 H), 1.86 (m, 1 H).

Two enantiomers of this compound can be separated using Chiral OD column using IPA/hexane (75/25) as the solvent.

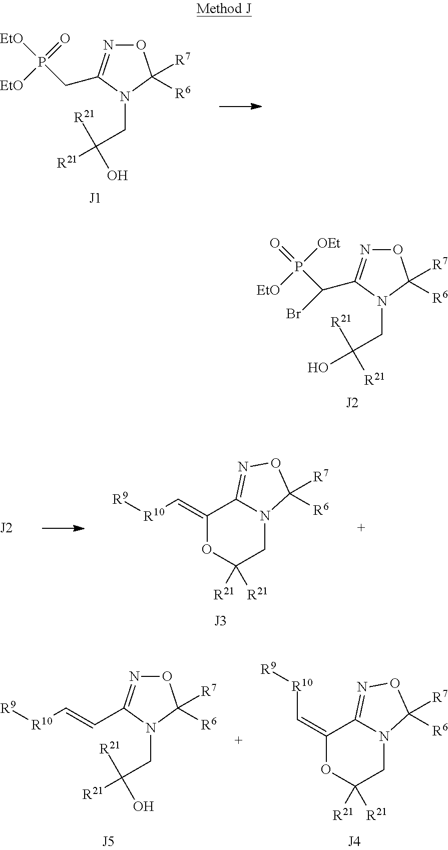

### Method J, Step 1

NBS (421 mg, 2.4 mmol) was added to a solution of J1 (R⁶=Me, R⁷=p-F-Phenyl, R²¹═R²¹=Me, obtained using method similar to that led to 12, 889 mg, 2.2 mmol) in CCl₄ (12 mL), and the reaction solution was stirred at room temperature for one hour. A catalytic amount of benzoyl peroxide (52 mg, 0.22 mmol) was added and the reaction solution was stirred at 60° C. for 12 hours. The reaction solution was clarified by filtration and the filtrate was concentrated under reduced pressure. The residue was diluted with ethyl acetate, and washed with saturated solution of sodium thiosulfate, and brine. The organic phase was dried over anhydrous sodium sulfate and concentrated under reduced pressure to obtain 1.0 g of product J2 (R⁶=Me, R⁷=p-F-Phenyl, R²¹═R²¹=Me) as a 1:1 mixture, which was used as is in the next reaction.

### Method J, Step 2

To a solution of J2 (R⁶=Me, R⁷=p-F-Phenyl, R²¹═R²¹=Me, 1.0 g, 2.1 mmol) and A1 (R¹⁰=3-MeO-Phenyl, R⁹=4-(4-Methylimidazol-1-yl) and R⁸═H, 427 mg, 1.98 mmol) [US 2007/0219181, page 62] in THF (40 mL) at room temperature was added sodium hydride (238 mg, 5.94 mmol) all at once, and the reaction solution was stirred at room temperature for 12 hours. The reaction solution was quenched with water, and extracted with ethyl acetate. The organic phase was dried over anhydrous sodium sulfate and concentrated under reduced pressure. The residue was dissolved in DMF (10 mL) and treated with sodium hydride (476 mg, 11.9 mmol). The reaction solution was stirred at room temperature for one hour before it was quenched with water. The layers ware separated and the organic layer was washed with water, dried over anhydrous sodium sulfate and concentrated. The residue was purified by column chromatography using Silica Gel (hexane:triethylamine=99:1) to obtain products J3 (R⁶=Me, R⁷=p-F-Phenyl, R²¹═R²¹⁼Me, R¹⁰=3-MeO-Phenyl, R⁹=4-(4-Methylimidazol-1-yl)), J4 (R⁶=Me, R⁷=p-F-Phenyl, R²¹═R²¹=Me, R¹⁰=3-MeO-Phenyl, R⁹=4-(4-Methylimidazol-1-yl)) and J5 (R⁶=Me, R⁷=p-F-Phenyl, R²¹═R²¹=Me, R¹⁰=3-MeO-Phenyl, R⁹=4-(4-Methylimidazol-1-yl)) in a ratio of 2:1:1, respectively.

¹H NMR (CDCl₃, ppm) of J3: δ 7.70 (s, 1 H), 7.56 (m, 2H), 7.49 (s, 1 H), 7.26 (m, 1 H), 7.18 (d, 1 H, J=8.0 Hz), 7.09 (m, 2H), 6.92 (s, 1 H), 6.55 (s, 1 H), 3.84 (s, 3H), 3.13 (d, 1 H, J=11.2 Hz), 2.78 (d, 1 H, J=11.2 Hz), 2.88 (s, 3H), 1.88 (s, 3H), 1.49 (s, 3H), 1.38 (s, 3H). MS (ES-LCMS, M+1) 463.3.

¹H NMR (CDCl₃, ppm) of J4: δ 7.89 (s, 1 H), 7.70 (s, 1 H), 7.54 (m, 2H), 7.18 (m, 2H), 7.08 (m, 2H), 6.92 (s, 1 H), 6.49 (s, 1 H), 3.90 (s, 3H), 3.06 (d, 1 H, J=10.8 Hz), 2.72 (d, 1 H, J=10.4 Hz), 3.90 (s, 3H), 1.86 (s, 3H), 1.40 (s, 3H), 1.30 (s, 3H). MS (ES-LCMS, M+1) 463.3.

¹H NMR (CDCl₃, ppm) J5: δ 7.70 (s, 1 H), 7.59-7.62 (m, 2H), 7.50 (d, 1 H, J=16.8 Hz), 7.22 (d, 1 H, J=8.4 Hz), 7.14 (d, 1 H, J=8.0 Hz), 6.06-7.10 (m, 3H), 6.92 (s, 1 H), 6.70 (d, 1 H, J=16.0 Hz), 3.86 (s, 3H), 3.01 (s, 2H), 2.29 (s, 3H), 1.93 (s, 3H), 1.62 (br, 1 H), 1.17 (s, 3H), 0.96 (s, 3H). MS (ES-LCMS, M+1) 465.3.

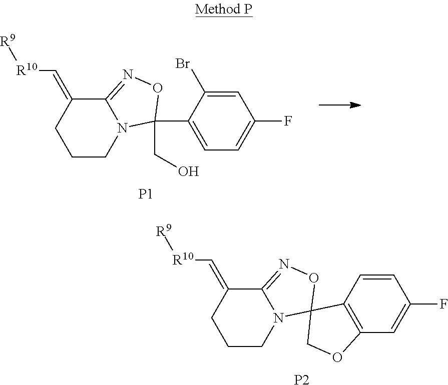

P1, obtained using method similar to method B and C, (0.19 g, 0.36 mmole, R¹⁰=3-MeO-Phenyl, R⁹=4-(4-Methyl-imidazol-1-yl)), palladium acetate (40 mg, 0.18 mmole), cesium carbonate (0.18 g, 0.54 mmole) and 1,1′-binaphthyl-2-yl-di-tert-butylphosphine (72 mg, 0.18 mmole) were placed in 100 ml RB flask before 15 ml dry toluene was added and the reaction was stirred at 90° C. overnight. Additional 0.5 eq. palladium acetate was added and the reaction was heated to 90° C. overnight. The reaction was cooled to room temperature before 100 ml EtOAc and 100 ml brine were added. The organic layer was washed with brine (2×100 ml), dried with Na2SO4 and concentrated. The product was purified by first with silica gel chromatograph eluted with DCM/MeOH followed by preparative TLC using EtOAc/Methanol/NEt3 as eluant and further followed by reverse phase HPLC to give pure P2 (R¹⁰=3-MeO-Phenyl, R⁹=4-(4-Methyl-imidazol-1-yl), 4.9 mg, 3%).

¹H NMR (CDCl₃, ppm) (R¹⁰=3-MeO-Phenyl, R⁹=4-(4-Methyl-imidazol-1-yl)): δ 7.72 (s, 1 H), 7.52 (s, 1 H), 7.40 (dd, 1 H), 7.26 (d, 1 H), 7.02 (d, 1 H), 6.99 (s, 1 H), 6.93 (s, 1 H), 6.72 (m, 1 H), 6.60 (dd, 1 H), 4.63 (dd, 2H), 3.10 (m, 1 H), 2.93 (m, 1 H), 2.77 (m, 2H), 2.30 (m, 3H), 1.91 (m, 2H).

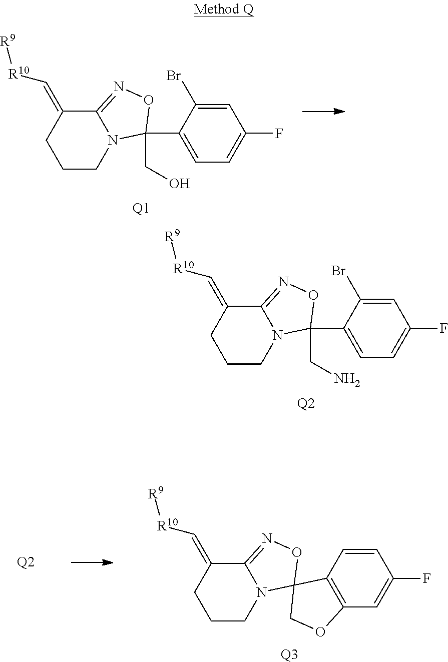

### Method Q, Step 1

Q1, synthesized using method similar to method B and C, (1 g, 1.9 mmole, R¹⁰=3-MeO-Phenyl, R⁹=4-(4-Methyl-imidazol-1-yl)), and Phthalimide (0.84 g, 5.7 mmole) was placed in a flask before tributylphosphine (1.15 g, 5.7 mmole), DEAD (0.99 g, 5.7 mmole) and 20 ml of toluenewere were added. The reaction was heated to 70° C. overnight with additional tributylphosphine (1.15 g, 5.7 mmole) and DEAD (0.99 g, 5.7 mmole) added and reaction heated to 70° C. for 6 hours. The reaction was cooled to room temperature before 100 ml EtOAc and 100 ml water were added. The organic layer was washed with water (100 ml), dried over Na₂SO₄ and concentrated. The residue was purified using a silica gel column eluted with DCM/MeOH. To the desired product dissolved in 10 ml DCM and 10 ml MeOH was added hydrazine (0.5 ml) and the reaction was heated to 40° C. for five hours before 50 ml EtOAc and 50 ml water were added. The organic layer was washed with brine (20 ml), dried with Na₂SO₄ and concentrated. The product was purified with silica gel column chromatograph eluted with DCM/MeOH. The fraction contains desired product was further purified by preparative TLC (DCM/MeOH) to give pure Q2 (R¹⁰=3-MeO-Phenyl, R⁹=4-(4-Methyl-imidazol-1-yl), 40 mg, 4% for two steps).

¹H NMR (CDCl₃, ppm) (R¹⁰=3-MeO-Phenyl, R⁹=4-(4-Methyl-imidazol-1-yl)): δ 7.71 (s, 1 H), 7.57 (dd, 1 H), 7.48 (s, 1 H), 7.45 (dd, 1 H), 7.24 (d, 1 H), 7.06 (m, 1 H), 7.00 (d, 1 H), 6.98 (s, 1 H), 6.92 (s, 1 H), 3.84 (s, 3H), 3.57 (dd, 2H), 3.28 (m, 1 H), 2.98 (m, 1 H), 2.74 (m, 2H), 2.29 (s, 3H), 1.92 (m, 2H).

### Method Q, Step 2

Q2 (40 mg, 0.076 mmole, R¹⁰=3-MeO-Phenyl, R⁹=4-(4-Methyl-imidazol-1-yl)), CuI (14 mg, 0.076 mmole) and K₂CO₃ were placed in a RB flask before N,N′-dimethylethlenediamine (13.4 mg, 0.152) in 20 ml toluene was added and the reaction was heated to 45° C. for five hours. The reaction was cooled to room temperature before 50 ml EtOAc and 50 ml water were added. The organic layer was washed with water (2×50 ml), dried over Na₂SO₄ and concentrated. The residue was purified with preparative TLC eluted with DCM/MeOH followed by reverse phase HPLC purification to give Q3 (R¹⁰=3-MeO-Phenyl, R⁹=4-(4-Methyl-imidazol-1-yl), 3.2 mg).

¹H NMR (CDCl₃, ppm) (R¹⁰=3-MeO-Phenyl, R⁹=4-(4-Methyl-imidazol-1-yl)): δ 7.74 (s, 1 H), 7.52 (s, 1 H), 7.34 (dd, 1 H), 7.26 (d, 1 H), 7.02 (d, 1 H), 7.99 (s, 1 H), 6.94 (s, 1 H), 6.51 (m, 1 H), 6.39 (dd, 1 H), 3.86 (s, 3H), 3.83 (m, 2H), 3.11 (m, 1 H), 2.93 (m, 1 H), 2.75 (m, 2H), 2.30 (s, 3H), 1.89 (m, 2H).

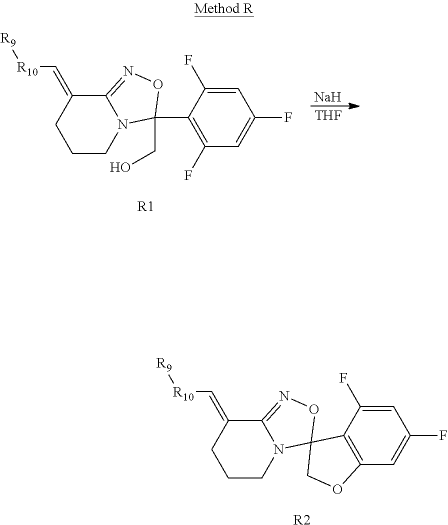

R1 (R¹⁰=3-MeO-Phenyl, R⁹=4-(4-Methyl-imidazol-1-yl)), obtained using methods similar to method B and C, was dissolved in 25 mL of THF. The solution was cooled to 0° C. before sodium hydride (15.8 mg, 60% dispersion in mineral oil) was added and the reaction was allowed to slowly warm to room temperature and stirred for two nights. The reaction was quenched with water, washed with ethyl acetate. The organic layer was washed with brine, dried over sodium sulfate and concentrated. The residue was purified with silica gel chromatograph eluted with DCM/MeOH yield 116.5 mg of R² (R¹⁰=3-MeO-Phenyl, R⁹=4-(4-Methyl-imidazol-1-yl)).

¹H NMR (CDCl3, ppm): δ 7.69 (s, 1 H), 7.47 (s, 1 H), 7.22 (d, J=8.1 Hz, 1 H), 6.99 (d, J=8.8 Hz, 1 H), 6.96 (s, 1 H), 6.91 (s, 1 H), 6.43-6.37 (m, 2H), 4.69 (d, J=11.7 Hz, 1 H), 4.56 (d, J=11.7 Hz, 1 H), 3.83 (s, 3H), 3.13-3.06 (m, 1 H), 2.96-2.83 (m, 2H), 2.58-2.48 (m, 1 H), 2.26 (s, 3H), 2.01-1.79 (m, 2H). MS (LCMS, M+1) 465.

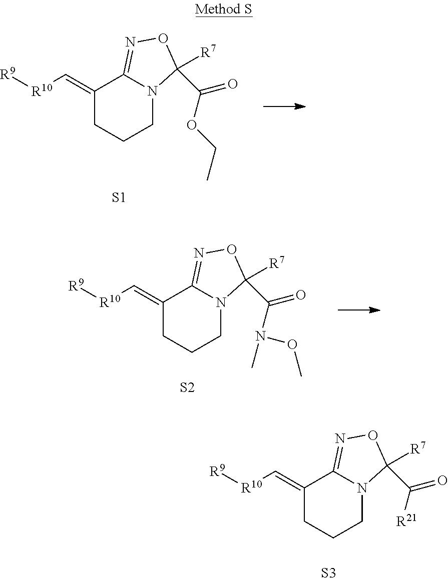

### Method S, Step 1

To a suspension of N,O-dimethylhydroxylamine hydrochloride (526 mg, 5.39 mmol) in DCM (4 ml), was added 2.70 m1 (5.39 mmol) of 2M AlMe₃ in toluene at 0° C. The mixture was stirred for 30 min. at ambient temperature, re-cooled to 0° C. and treated with a solution of S1, obtained using method similar to method B, (R⁷=3,5-di-F-Phenyl, R¹⁰=3-MeO-Phenyl, R⁹=4-(4-Methyl-imidazol-1-yl)) in DCM (2 ml). The mixture was stirred at ambient temperature over 1-2 days, quenched at 0° C. by dropwise addition of excess of 1M tartaric acid, and extracted by DCM (3×). The product was purified by chromatography on 24 g of SiO₂ using a gradient of 0-8% of MeOH in DCM to furnish S2 (R⁷=3,5-di-F-Phenyl, R¹⁰=3-MeO-Phenyl, R⁹=4-(4-Methyl-imidazol-1-yl)), ¹H NMR (CDCl₃, ppm): δ 7.70 (s, 1 H), 7.50 (s, 1 H), 7.24 (m, 1 H), 7.06-6.81 (m, 6H), 3.84 (s, 3H), 3.77 (m, 1 H), 3.64 (s, 3H), 3.22 (s, 3H), 2.77 (m, 2H), 2.62 (m, 1 H), 2.29 (s, 3H), 1.99 (m, 1 H), 1.74 (m, 1 H). LCMS (MH⁺)=524.2.

Two enantiomers of this compound can be separated using Chiral AD column using IPA/hexane (70/30) as the solvent to furnish (−)-enantiomer A of S2-1 (203 mg), and (+)-enantiomer B of S2-2 (200 mg).

### Method S, Step 2

To a solution of (−)-enantiomer A of S2-1 (R⁷=3,5-di-F-Phenyl, R¹⁰=3-MeO-Phenyl, R⁹=4-(4-Methyl-imidazol-1-yl)), in THF (3 mL) at −78° C. was added 0.21 mL (0.573 mmol) of 3M solution of MeMgBr in ether. The solution was stirred for 30 min and was allowed to warm up to ambient temperature. The reaction mixture was quenched with water and extracted with DCM. The product was purified by silica gel chromatography to furnish 156 mg of S3, (R⁷=3,5-di-F-Phenyl, R¹⁰=3-MeO-Phenyl, R⁹=4-(4-Methyl-imidazol-1-yl), R²¹=Me),

¹H NMR (CDCl₃, ppm) of the enantiomer A of S3 (R⁷=3,5-di-F-Phenyl, R¹⁰=3-MeO-Phenyl, R⁹=4-(4-Methyl-imidazol-1-yl) and R²¹=Me), ¹H NMR (CDCl₃, ppm): δ 7.71 (s, 1 H), 7.50 (s, 1 H), 7.26 (s, 1 H), 7.07-6.85 (ser. m., 6H), 3.85 (s, 3H), 3.62 (m, 1 H), 2.91 (m, 1 H), 2.79-2.59 (m, 1 H), 2.31 (s, 3H), 2.29 (s, 3H), 1.94 (m, 1 H), 1.78 (m, 1 H). LCMS (MH⁺)=479.2; retention time=2.062 min (gradient A).

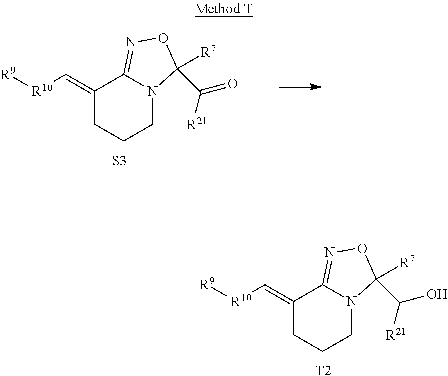

To a mixture of S3 (R⁷=3,5-di-F-Phenyl, R¹⁰=3-MeO-Phenyl, R⁹=4-(4-Methyl-imidazol-1-yl) and R²¹=Me; 136 mg) in 2.5 mL of THF was added 0.22 mL of 1.0 M solution of (S)-2-methyl-CBS-oxazaborolidine in toluene followed by 0.23 mL of 2M borane-dimethylsulfide complex in THF. The reaction mixture was stirred overnight, quenched with water, extracted with ethyl acetate, and concentrated to give a diastereomeric mixture of alcohol T2. The diastereomeric alcohols T2 (R⁷=3,5-di-F-Phenyl, R¹⁰=3-MeO-Phenyl, R⁹=4-(4-Methyl-imidazol-1-yl) and R²¹=Me) were separated by reverse-phase chromatography on a C-18 Column using a gradient of water-acetonitrile with 0.1% of TFA as a modifier. ¹H NMR (CDCl₃, ppm) of the minor diastereomer (11 mg obtained) of product T2-1 (R⁷=3,5-di-F-Phenyl, R¹⁰=3-MeO-Phenyl, R⁹=4-(4-Methyl-imidazol-1-yl) and R²¹=Me): δ 7.80 (s, 1 H), 7.47 (s, 1 H), 7.24-7.18 (ser m, 3H), 7.01-6.92 (ser m, 3H), 6.83 (s, 1 H), 4.51 (m, 1 H), 3.85 (s, 3H), 3.32 (m, 1 H), 2.95 (m, 1 H), 2.79 (m, 1 H), 2.54 (m, 1 H), 2.30 (s, 3H), 1.86 (m, 2H), 1.38 (d, J=6.4 Hz, 3H). LCMS (MH⁺)=481.2; retention time=3.22 min (gradient B).

¹H NMR (CDCl₃, ppm) of the major diastereomer 2 (25 mg obtained) of product T2-2, (R⁷=p-F-Phenyl, R¹⁰=3-MeO-Phenyl, R⁹=4-(4-Methyl-imidazol-1-yl), and R²¹=Me): δ 7.74 (s, 1 H), 7.45 (s, 1 H), 7.24 (m, 1 H), 7.06 (m, 2H), 6.96 (m, 3H), 6.83 (m, 1 H), 4.44 (m, 1 H), 3.84 (s, 3H), 3.45 (m, 1 H), 3.07 (m, 1 H), 2.79 (m, 1 H), 2.55 (m, 1 H), 2.30 (s, 3H), 1.91 (m, 2H), 1.17 (d, J=6.4 Hz, 3H). LCMS (MH⁺)=481.2; retention time=3.34 min (gradient B).

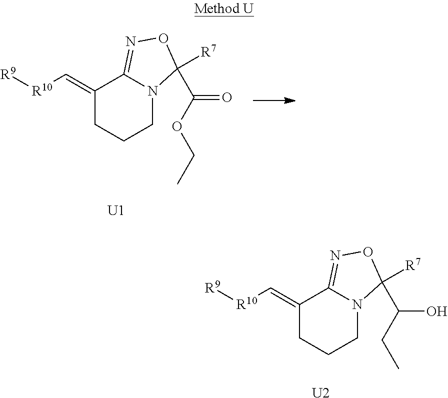

To a solution of 200 mg of U1, obtained using a method similar to method B, (R⁷=3,5-di-F-Phenyl, R⁶=carboethoxyl, R¹⁰=3-MeO-Phenyl, R⁶=4-(4-Methyl-imidazol-1-yl)) in 2.0 mL of THF was added 35 μL of titanium tetraisopropoxide. The mixture was chilled to 0° C., and 0.39 mL of 3M solution of ethylmagnesium bromide in ether was added dropwise. The mixture was quenched with water, extracted with DCM, and the product was purified chromatographically (SiO₂) using a gradient of MeOH in DCM (from 0 to 9%) as the solvent to furnish 20 mg of U2 (R⁷=3,5-di-F-Phenyl, R¹⁰=3-MeO-Phenyl, R⁹=4-(4-Methyl-imidazol-1-yl) and R²¹=Et), ¹H NMR (CDCl₃, ppm): δ 7.73 (s, 1 H), 7.46 (s, 1 H), 7.25-7.17 (ser m, 3H), 6.95 (ser m, 3H), 6.82 (m, 1 H), 4.20-4.14 (m, 1 H), 3.84 (s, 3H), 3.35 (m, 1 H), 2.95 (m, 1 H), 2.82-2.74 (m, 1 H), 2.57-2.49 (m, 1 H), 2.30 (s, 3H), 1.85 (m, 2H), 1.79-1.70 (m, 1 H), 1.63-1.52 (m, 1 H), 1.09 (t, J=7.3 Hz, 3H). LCMS (MH⁺)=495.5; retention time=2.002 min (gradient A).

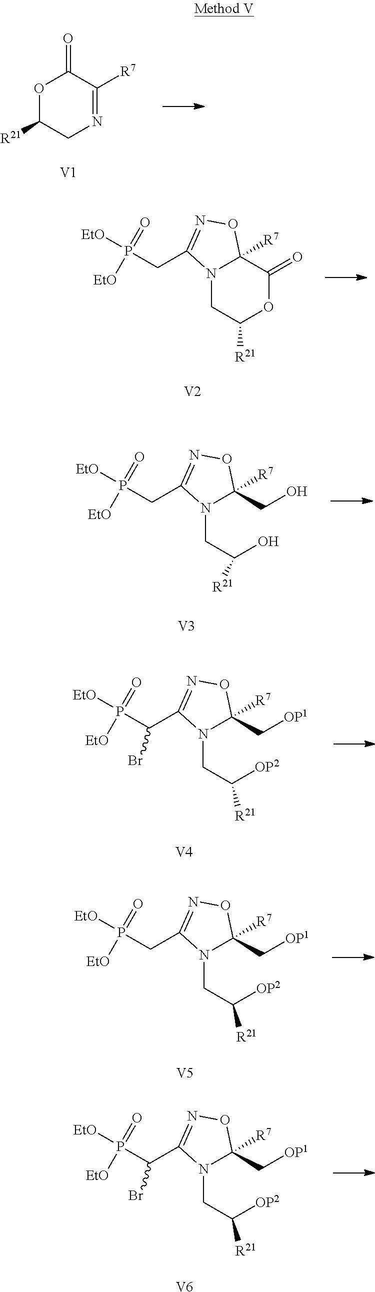

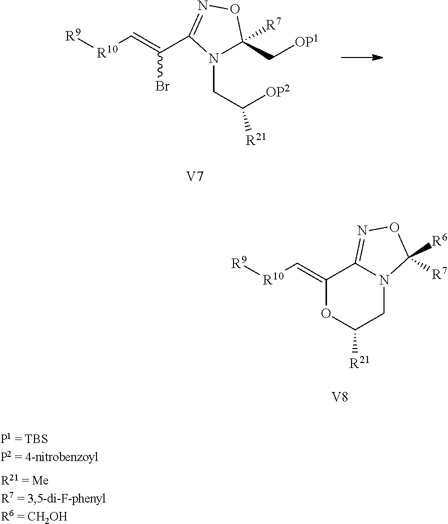

### Method V, Step 1

Compound V1 (R²¹=Me, R⁷=3,5-di-F-phenyl) was prepared according to Bieckert et al, Chem. Ber. 1961, 94, 2785 (Chem. Abstr. 56:31409) and converted to compound V2 (R²¹=Me, R⁷=3,5-di-F-phenyl) according to Method B, Step 3. LCMS (MH⁺)=419.2, (MNa⁺)=441.2, (2MNa⁺)=859.0; retention time=2.057 min (gradient A).

### Method V, Step 2

Lactone ring of compound V2 (R²¹=Me, R⁷=3,5-di-F-phenyl) was reduced to the diol V3 (R²¹=Me, R⁷=3,5-di-F-phenyl) according to the procedure of Method C, LCMS (MH⁺)=423.2; retention time=1.87 min (gradient A).

### Method V, Step 3

A mixture of 5.24 g (12.4 mmol) of the dial V3 (R²¹=Me, R⁷=3,5-di-F-phenyl) and 1.689 g (24.8 mmol) of imidazole in 62 mL of DMF was cooled with ice and treated dropwise with a mixture of 2.244 g of TBSCI in 62 mL of DMF. The reaction mixture was stirred overnight over which period of time it was allowed to warm up to room temperature, diluted with 200 mL of ethyl acetate and washed with 200 mL of water. The aqueous phase was extracted 2×100 mL of ethyl acetate. Combined organic phase was washed with brine and dried over Na₂SO₄ and concentrated, and the product was isolated by flash chromatography using a gradient 0-50% of ethyl acetate in hexanes as the solvent to furnish 3.76 g of V4 (R²¹=Me, R⁷=3,5-di-F-phenyl, P¹=TBS), LCMS (MH⁺)=536.9; retention time=2.46 min (gradient A).

### Method V, Step 4

A mixture of 3.76 g of V4 (R²¹=Me, R⁷=3,5-di-F-phenyl, P¹=TBS) and 3.678 g (14 mmol) of PPh₃ in 28 mL of THF was cooled to 0° C. before a solution of 2.344 g (14 mmol) of 4-nitrobenzoic acid in 42 mL of THF was added followed by addition of 2.21 mL (14 mmol) of DEAD and the reaction was stirred at ambient temperature for 1 h before it was diluted with 200 mL of EtOAc, washed with 100 mL of sat NaHCO₃, 50 mL of water, dried over Na₂SO₄ and solvent evaporated to approx 30 mL followed by addition of 10 mL of hexanes to precipitate the by-product triphenylphospine oxide. The mixture was filtered, filtrate concentrated, and the residue purified by flash chromatography using a gradient 0-50% of ethyl acetate in hexanes to furnish NMR 4.88 g of V5 (R²¹=Me, R⁷=3,5-di-F-phenyl, P¹=TBS, P²=4-nitrobenzoyl), LCMS (MH⁺)=685.8; retention time=2.74 min (gradient A).

### Method V, Step 5

V5 (R²¹=Me, R⁷=3,5-di-F-phenyl, P¹=TBS, P²=4-nitrobenzoyl) was converted to V6 (R²¹=Me, R⁷=3,5-di-F-phenyl, P¹=TBS, P²=4-nitrobenzoyl) using method similar J. Diastereomer V6-1, LCMS (MH⁺)=763.6 and 765.6; retention time=2.85 min, Diastereomer V6-2, LCMS (MH⁺)=763.6 and 765.6; retention time=2.88 min (gradient A)

### Method V, Step 6

V6-1 (R²¹=Me, R⁷=3,5-di-F-phenyl, P¹=TBS, P²=4-nitrobenzoyl) was converted to V7 using a method similar to method B (R²¹=Me, R⁷=3,5-di-F-phenyl, R¹⁰=3-MeO-Phenyl, R⁹=4-(4-Methyl-imidazol-1-yl, P¹=TBS, P²=4-nitrobenzoyl), LCMS (MH⁺)=826.2; retention time=2.55 min (gradient A).

### Method V, Step 7

(a) To accomplish cleavage of P¹ and P² groups, a mixture of 165 mg (0.2 mmol) of Compound V7 (R²¹=Me, R⁷=3,5-di-F-phenyl, R¹⁰=3-MeO-Phenyl, R⁹=4-(4-Methyl-imidazol-1-yl, P¹=TBS, P²=4-nitrobenzoyl) was dissolved in 10 mL of MeOH and treated with 165 mg of K₂CO₃, and the resulting suspension was stirred overnight. The reaction was quenched with water and extracted with ethyl acetate. The organic phase was washed with brine, dried over sodium sulfate, and concentrated.

(b) To accomplish cyclization, 60 mg of the material obtained in (a) was dissolved in 1 mL of 1:1 mixture of THF and DMF and treated with 10 mg of sodium hydride (60% dispersion in mineral oil). The mixture was stirred for 30 min, quenched with water and extracted with ethyl acetate. The organic phase was dried over sodium sulfate and concentrated. The residue was isolated by reverse-phase chromatograpy on C-18 phase using a gradient of water-acetonitrile with 0.1% TFA as an additive to yield 10 mg of V8 (R²¹=Me, R⁷=3,5-di-F-phenyl, R⁷═CH2OH, R¹⁰=3-MeO-Phenyl, R⁹=4-(4-Methyl-imidazol-1-yl)), NMR (CDCl₃, ppm): δ 7.72 (s, 1 H), 7.46 (s, 1 H), 7.24 (m, 1 H), 7.19 (m, 1 H), 7.03 (m, 2H), 6.92 (s, 1 H), 6.85 (m, 1 H), 6.41 (m, 1 H), 4.44 (m, 1 H), 4.17-4.08 (ser m, 2H), 4.00 (d, J=13.0 Hz, 1 H), 3.82 (s, 3H), 3.43 (dd, J=8.2, 3.0 Hz, 1 H), 3.08 (dd, J=9.7, 11.0 Hz, 1 H), 2.28 (s, 3H), 1.48 (d, J=6.4 Hz, 3H); LCMS (MH⁺)=483.2; retention time=1.97 min.

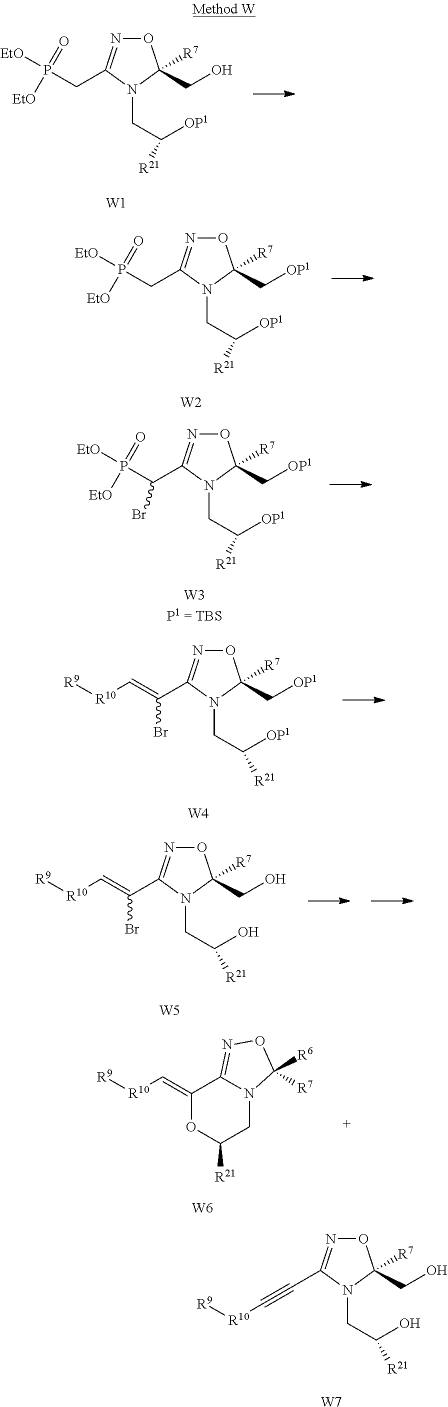

### Method W, Step 1

Compound W1, obtained using method similar to method V (R²¹=Me, R⁷=3,5-di-F-phenyl) was converted into bis-TBS ether W2 (R²¹=Me, R⁷=3,5-di-F-phenyl). LCMS (MH⁺)=651.2; retention time=3.25 min.

### Method W, Step 2

Compound W2 (R²¹=Me, R⁷=3,5-di-F-phenyl) was brominated using a method similar to method J to furnish compound W3 (R²¹=Me, R⁷=3,5-di-F-phenyl). Diastereomer W3-1, LCMS (MH⁺)=729.2; retention time=3.46 min, Diastereomer W3-2, LCMS (MH⁺)=729.2; retention time=3.55 min.

### Method W, Step 3

Using compound W3-2 (R²¹=Me, R⁷=3,5-di-F-phenyl) was converted to W4 (R²¹=Me, R^{7=3,5}-di-F-phenyl, R¹⁰=3-MeO-Phenyl, R⁹=4-(4-Methyl-imidazol-1-yl, P¹═P²=TBSI)) using a method similar to method B, LCMS (MH⁺)=790.28; retention time=3.43 min.

### Method W, Step 4

W4 (R²¹=Me, R⁷=3,5-di-F-phenyl, R¹⁰=3-MeO-Phenyl, R⁹=4-(4-Methyl-imidazol-1-yl, P¹═P²=TBSI) was treated with TBAF according to procedure of Method K, Step 3, to furnish W5 (R²¹=Me, R⁷=3,5-di-F-phenyl, R¹⁰=3-MeO-Phenyl, R⁹=4-(4-Methyl-imidazol-1-yl)), LCMS (MH⁺)=563.0; retention time=1.97 min.

### Method W, Step 5

To a solution of 255 mg of W5 (R²¹=Me, R⁷=3,5-di-F-phenyl, R¹⁰=3-MeO-Phenyl, R⁹=4-(4-Methyl-imidazol-1-yl)) in 8.0 mL of 1:1 mixture of DMF and THF at 0° C. was added 39 mg of 60% suspension of NaH in mineral oil. The reaction was stirred for a period of 1 hr 40 min, quenched with water, and extracted with EtOAc. The organic phase was washed with brine, dried over Na₂SO₄ and concentrated. The residue was purified by flash chromatography using a gradient of 0-50% of acetonitrile in DCM, to provide a mixture of compounds W6 (R²¹=Me, R⁷=3,5-di-F-phenyl, R⁶═CH2OH, R¹⁰=3-MeO-Phenyl, R⁹=4-(4-Methyl-imidazol-1-yl). The components of the mixture were separated by chromatography on AD column using 40-60% of IPA in hexanes as the solvent to furnish 108 mg of W6-1 (R²¹=Me, R⁷=3,5-di-F-phenyl, R⁶=CH2OH, R¹⁰=3-MeO-Phenyl, R⁹=4-(4-Methyl-imidazol-1-yl)), NMR (CDCl₃, ppm): δ 7.72 (s, 1 H), 7.48 (s, 1 H), 7.27-7.24 (ser m, 2H), 7.08 (m, 2H), 6.93 (s, 1 H), 6.87 (m, 1 H), 6.46 (s, 1 H), 4.33 (m, 1 H), 4.11 (s, 2H), 3.86 (s, 3H), 3.43 (t, J=9.7 Hz, 1 H), 3.13 (dd, J=3.0, 11.0 Hz, 1 H), 2.29 (s, 3H), 1.51 (d, J=6.2 Hz, 3H). LCMS (MH⁺)=483.2; retention time=1.98 min; and 2.0 mg of W6-2 (R²¹=Me, R⁷=3,5-di-F-phenyl, R¹⁰=3-MeO-Phenyl, R⁹=4-(4-Methyl-imidazol-1-yl)), NMR (CDCl₃, ppm): δ 7.75 (s, 1 H), 7.24 (m, 2H), 7.15 (m, 2H), 7.06 (m, 2H), 6.93 (s, 1 H), 4.21 (d, J=13.2 Hz, 1 H), 4.10 (d, J=13.2 Hz, 1 H), 3.87 (s, 3H), 3.48 (dd, J=1.8, 15.0 Hz, 1 H), 2.86 (dd, J=10.0, 15.5 Hz, 1 H), 2.28 (s, 3H), 1.20 (d, J=6.2 Hz, 3H). LCMS (MH⁺)=483.2; retention time=1.91 min.

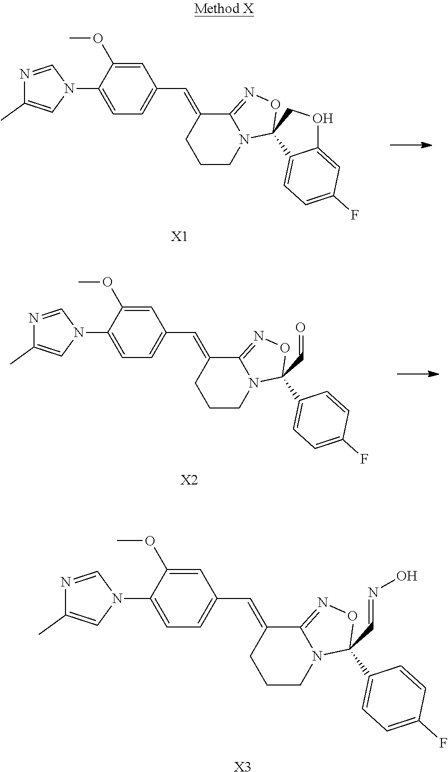

### Method X, Step 1

Anhydrous DMSO (0.633 mL) was added dropwise under nitrogen atmosphere to a solution of oxalyl chloride (0.453 mL) in 24.6 mL of anhydrous DCM at −78° C. The reaction mixture was stirred for 10 min at −78° C. before a solution of X1 (2.0 g) in 6.0 mL of anhydrous DCM was added dropwise. The reaction mixture was stirred 1.5 h before addition of TEA (2.5 mL) at −78° C., stirred for an additional 1 h between −78° C. and r.t., and diluted with 30 mL of water and extracted with EtOAc. The organic phase was dried over anhydrous magnesium sulfate, filtered and solvent evaporated to give 2.1 g of X2.

### Method X, Step 2

Potassium acetate (57.2 mg) in 0.6 mL of MeOH was added dropwise to a stirred solution of X2 (200 mg) in 2.2 mL of MeOH at 0° C. A solution of hydroxylamine hydrochloride (40.5 mg) in 0.6 mL of MeOH was then added. The reaction mixture was stirred at r.t. overnight then quenched with iced-brine and extracted with EtOAc. The organic phase was dried over anhydrous sodium sulfate, filtered and evaporated. The crude was purified via a reverse-phase column with MeCN/Water containing 0.1% formic acid to give 32.5 mg of X3.

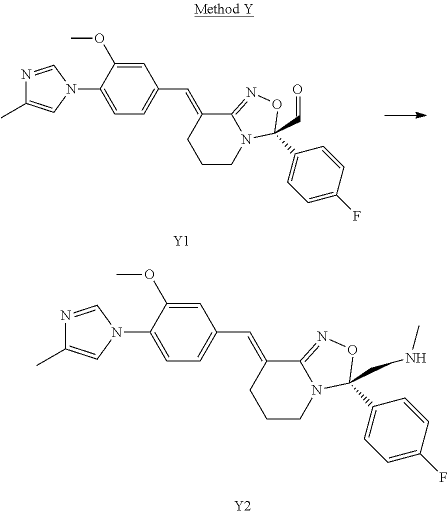

Solid sodium triacetoxy borohydride (252 mg) was slowly added to a stirred reaction mixture of Y1, obtained using a method similar to method X, (200 mg) methylamine (0.5 mL of 2M solution in THF), and acetic acid (0.8 mL of 1.12M solution in DCE) in 12 mL of DCE at 0° C. under nitrogen atmosphere. The reaction mixture was stirred at r.t. overnight, quenched with saturated aqueous sodium bicarbonate, and extracted with EtOAc. The organic phase was dried over anhydrous sodium sulfate and evaporated. Residue was purified via a reverse-phase column with MeCN/Water containing 0.1% formic acid to give 87.9 mg of Y2.

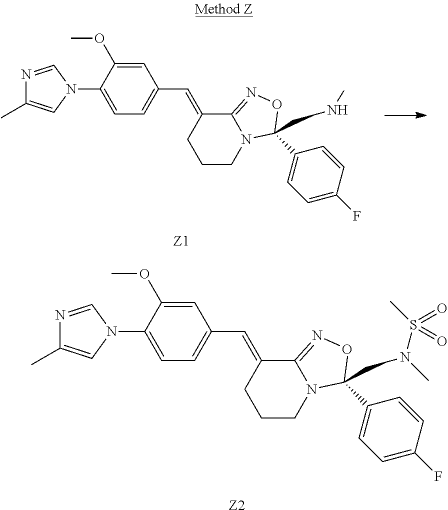

A solution of methanesulfonyl chloride (40.5 μL) was added dropwise to a stirred reaction mixture of Z1, obtained using a method similar to method Y, (190 mg) and TEA (172 μL) in 3.4 mL. of anhydrous DCM at 0° C. under nitrogen atmosphere. The reaction mixture was stirred 30 min at 0° C., then stirred 30 min between 0° C. and r.t. quenched with saturated aqueous sodium bicarbonate, and extracted with EtOAc. The organic phase was dried over anhydrous sodium sulfate and evaporated. Residue was purified via a reverse-phase column with MeCN/Water with 0.1% formic acid to give 54.5 mg of Z2.

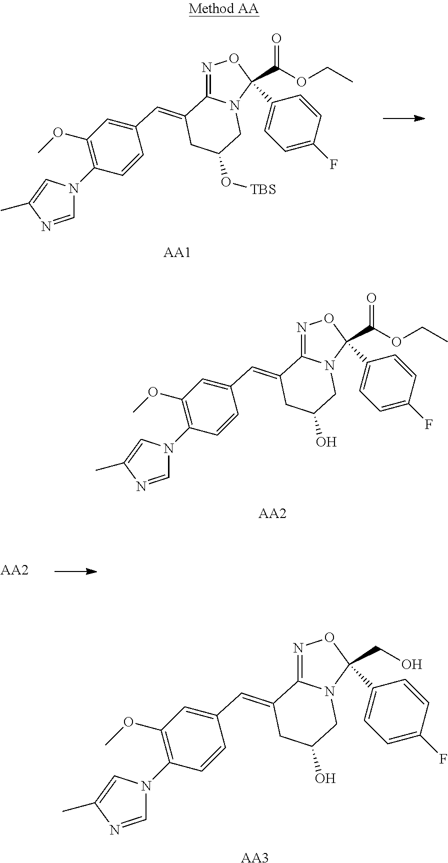

### Method AA, Step 1,

AA1, prepared from (S)-1-amino-3-bromopropan-2-ol hydrobromide using the procedure similar to method B, was treated with 1M tetrabutylammonium fluoride in THF to give product AA2.

### Method AA, Step 2,

AA3 was prepared from AA2 using the procedure similar to method C.

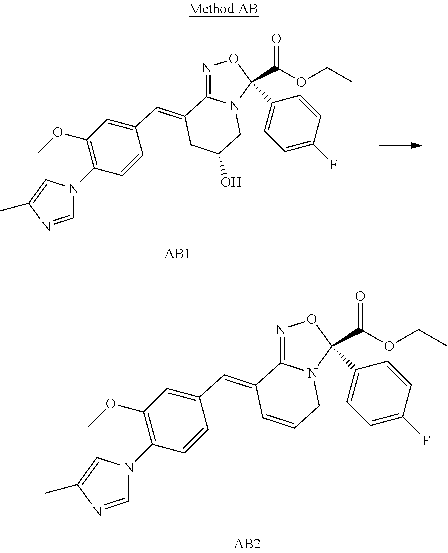

[Bis(2-methoxyethyl)-amino]sulfur trifluoride in 60 μl. anhydrous DCM was added to a solution of AB1 in 100 μL anhydrous DCM at −78° C. under N₂ protection. The reaction mixture was stirred at −78° C. for 1.5 h and at r.t. for additional 1.5 h. Then the reaction mixture was quenched with ice and aqueous NaHCO₃, extracted with EtOAC, and purified on a C18 Column to give AB2.

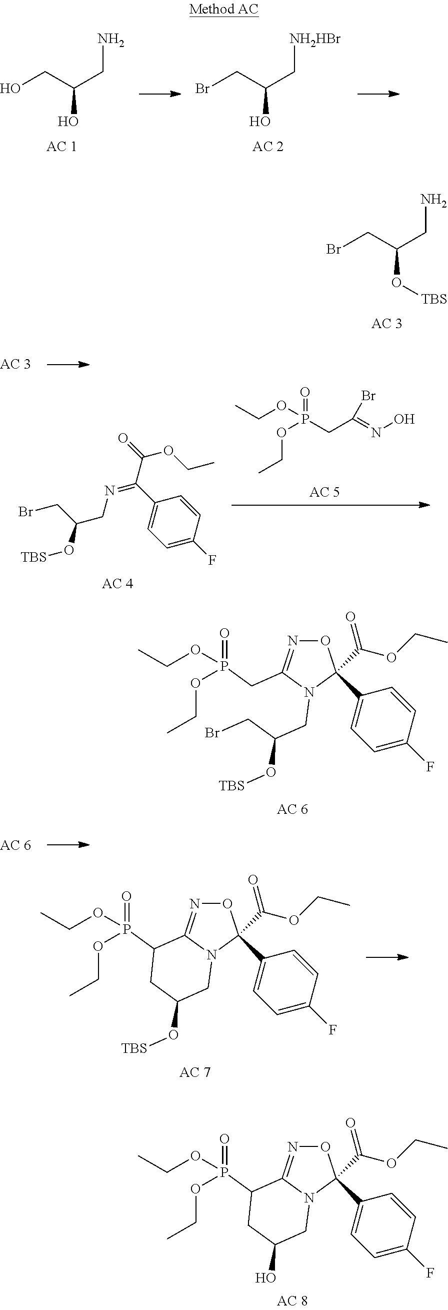

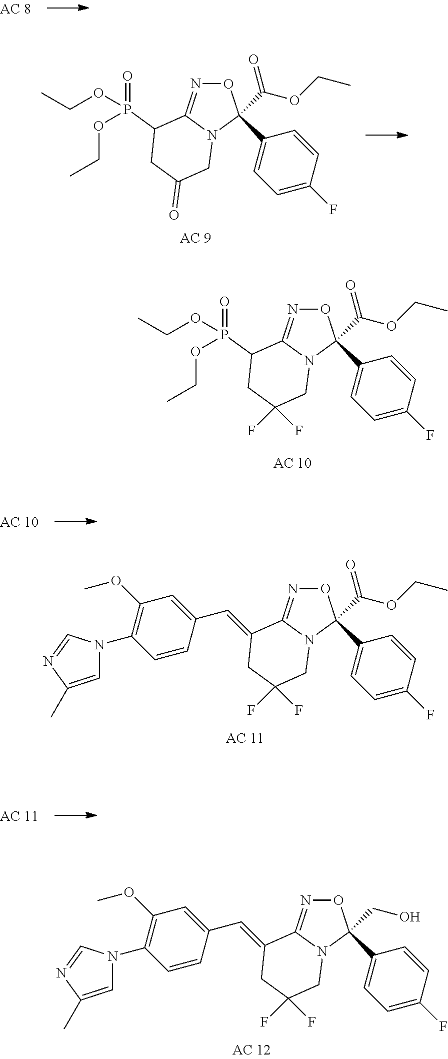

### Method AC, Step 1

AC2 was prepared from (R)-3-amino-1,2-propanediol (AC 1) using a similar method described in WO 2007/011162. AC1 (25 g) was treated with 45% HBr in HOAc at 40° C. for 3 h. Then the reaction solution was refluxed in 150 mL anhydrous EtOH for 3.5 h. After concentration, the residue was washed with ether to give AC 2, 63% yield.

### Method AC, Step 2

AC2 (5.1 g) was treated with 3.3 g TBS-Cl in 40 mL DCM and 7 mL Et₃N at r.t. overnight to give AC3, 91% yield.

### Method AC, Step 3

AC4 was prepared from AC3 and ethyl 4-fluorobenzoylformate using the procedure similar to method B.

### Method AC, Step 4

AC6 was prepared from AC4 and AC5 using a procedure similar to method

### Method AC, Step 5

AC7 was prepared from AC6 using a procedure similar to method B.

### Method AC, Step 6

AC7 was treated with 1.2 equiv of 1M tetrabutylammonium fluoride in THF for 40 min to give AC8.

### Method AC, Step 7

A solution of 72 mg of AC8 in 1 mL DCM was added to a mixture of Dess-Martin reagent in 0.2 mL DCM. The reaction mixture was stirred at r.t. for 2.5 h, and quenched with aqueous Na₂S₂O₃—NaHCO₃. The crude was purified on a silica gel column to give AC9, 65% yield.

### Method AC, Step 8

Under N₂ protection, 40 mg of [Bis(2-methoxyethyl)-amino]sulfur trifluoride in 21 μL of anhydrous DCM was added to a solution of 47 mg of AC9 in 30 μl. of anhydrous DCM at r.t. Then 1.2 μL of anhydrous EtOH in 32 μL of anhydrous DCM was added dropwise. The reaction solution was stirred at r.t. for 2 h, and quenched with a mixture of aqueous NaHCO₃ and ice. The crude was purified on a silica gel column to give AC10, 62% yield.

### Method AC, Step 9

AC11 was prepared from AC10 using a procedure similar to method B.

### Method AC, Step 10

AC12 was prepared from AC11 using a procedure similar to method C.

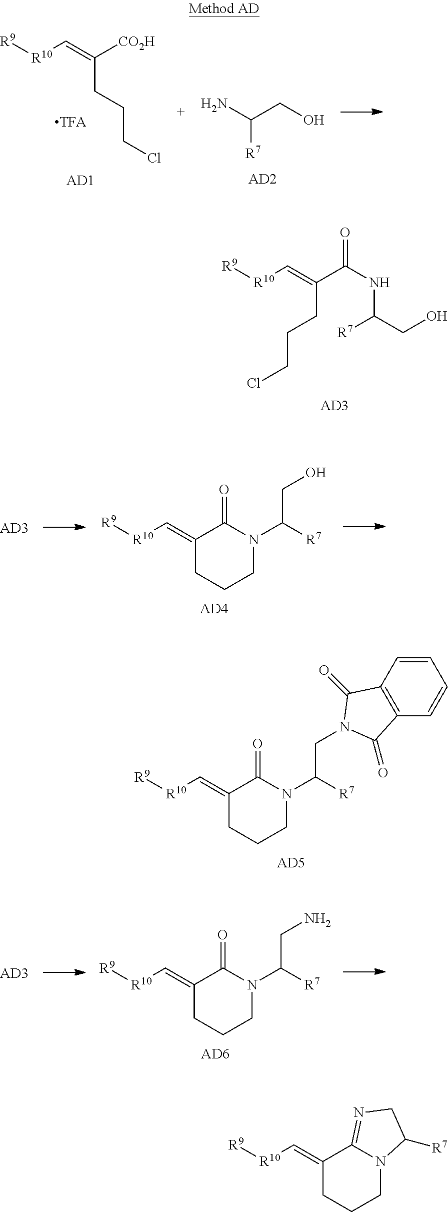

### Method AD, Step 1

To the solution of AD1 (R¹⁰=3-MeO-Phenyl, R⁹=4-(4-Methyl-imidazol-1-yl), 6.68 mmol, 3 g) and AD2 (R⁷=4-F-Phenyl, 7.35 mmol, 1.14 g) in DMF (30 ml) at room temperature was added DIPEA (23.4 mmol, 4 ml), HOBt (13.37 mmol, 1.81 g) and EDCI (13.37 mmol, 2.56 g) and allowed to stir overnight. The reaction was concentrated to remove DMF. The residue was dissolved in ethyl acetate and washed with water and brine. The combined organic extract was dried with anhydrous sodium sulfate. It was then filtered and concentrated to give 5.61 g of desired product AD3 (R⁷=4-F-Phenyl, R′⁹=3-MeO-Phenyl, R⁹=4-(4-Methyl-imidazol-1-yl)).

### Method AD, Step 2

To the solution of AD3 (R⁷=4-F-Phenyl, R¹⁰=3-MeO-Phenyl, R⁹=4-(4-Methyl-imidazol-1-yl), 11.9 mmol, 5.61 g) in THF at 0° C., NaH (29.7 mmol, 1.2 g) was added slowly. The reaction mixture was allowed to stir overnight at room temperature. The reaction was quenched using ammonium chloride solution at 0° C. It was then extracted three times with ethyl acetate. The combined organic extract was dried with anhydrous sodium sulfate. It was then filtered and concentrated. The residue was purified by silica gel chromatography using (0-5) MeOH/CH₂Cl₂ to give 1.61 g of AD4 (R⁷=4-F-Phenyl, R¹⁰=3-MeO-Phenyl, R⁹=4-(4-Methyl-imidazol-1-yl).

### Method AD, Step 3

To the solution of AD4 (R⁷=4-F-Phenyl, R¹⁰=3-MeO-Phenyl, R⁹=4-(4-Methyl-imidazol-1-yl), 2.3 mmol, 1.0 g) in 15 ml THF, was added ADDP (5.74 mmol, 1.45 g), nPBu₃ (5.74 mmol, 1.43 ml) and pthalimide (5.74 mmol, 861 mg). The reaction mixture was refluxed overnight at 80° C. The reaction mixture was cooled to room temperature quenched with water and extracted three times with ethylacetate. The combined organic extract was dried with anhydrous sodium sulfate. It was then filtered and concentrated. The residue was purified by silica gel chromatography using (0-5) % MeOH/CH₂Cl₂ to give 1.06 g of AD5 (R⁷=4-F-Phenyl, R¹⁰=3-MeO-Phenyl, R⁹=4-(4-Methyl-imidazol-1-yl)).

### Method AD, Step 4

To a solution of AD5 (R⁷=4-F-Phenyl, R¹⁰=3-MeO-Phenyl, R⁹=4-(4-Methyl-imidazol-1-yl), 1.88 mmol, 1.06 g) in MeOH/CH₂Cl₂ (10 ml/10 ml) hydrazine Hydrate (28.2 mmol, 0.9 ml) was added. The reaction mixture was allowed to stir overnight at room temperature. The reaction mixture was filtered. The filtrate was concentrated and purified by silica gel chromatography using (0-5) % (2N NH3/MeOH)/CH₂Cl₂ to give 224 mg of AD6 (R⁷=4-F-Phenyl, R¹⁰=3-MeO-Phenyl, R⁹=4-(4-Methyl-imidazol-1-yl)).

### Method AD, Step 5

In 25 ml of POCl₃, 224 mg of AD6 (R⁷=4-F-Phenyl, R¹⁰=3-MeO-Phenyl, R⁹=4-(4-Methyl-imidazol-1-yl)) was stirred at 60° C. overnight. The reaction was concentrated and the residue was dissolved in methylene chloride and washed with sodium bicarbonate solution. The combined organic extract was dried with anhydrous sodium sulfate. It was then filtered and concentrated. It was purified by silica gel chromatography using (0-5) % (2N NH₃/MeOH)/CH₂Cl₂ to give 131 mg of AD7 (R⁷=4-F-Phenyl, R¹⁰=3-MeO-Phenyl, R⁹=4-(4-Methyl-imidazol-1-yl)). ¹H NMR (CD3OD, ppm): δ 7.94-7.92 (m, 1 H), 7.79-7.76 (m, 1 H), 7.59-7.50 (m, 3H), 7.37-7.33 (m, 1 H), 7.32-7.24 (m, 3H), 7.19-7.16 (m, 1 H), 4.47-4.40 (m, 1 H), 3.98 (s, 3H), 3.45-3.36 (m, 1 H), 3.22-3.13 (m, 1 H), 3.02-3.00 (m, 1 H), 2.98-2.92 (m, 2H), 2.90-2.87 (m, 1 H), 2.28 (s, 3H), 2.08-1.93 (m, 2H); (ES-LCMS, M+1) 417.2. Retention time: 2.30 min.

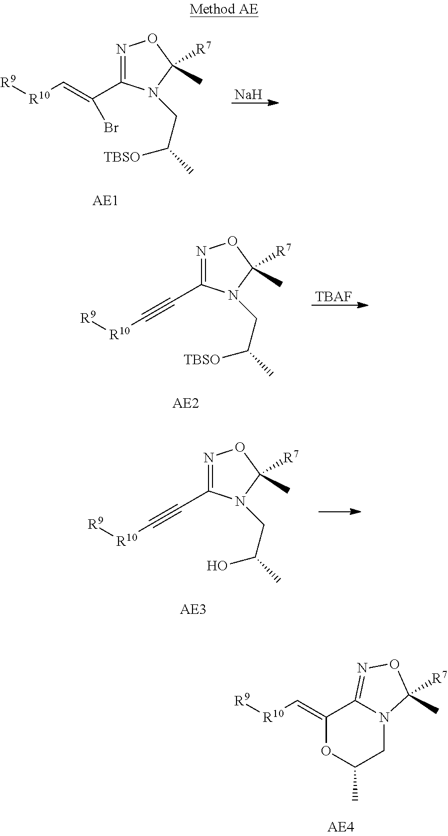

### Method AE, Step 1

NaH (17 mg, 0.43 mmol.) was added to a solution of AE1 (R7=4-F-Phenyl, R10=3-MeO-Phenyl, R9=4-(4-Methyl-imidazol-1-yl), 46 mg, 0.07 mmol.) in 2:1 mixture of THF and DMF (2 mL). After stirring at room temperature for 1 hour, the reaction mixture was quenched with water. The organic material was extracted with ethyl acetate, dried over anhydrous MgSO₄ and concentrated to give 33.2 mg of the title compound (R7=4-F-Phenyl, R10=3-MeO-Phenyl, R9=4-(4-Methyl-imidazol-1-yl)).

### Method AE, Step 2:

TBAF (0.2 mL, 0.2 mmol., 1M THF) was added to a solution of compound AE2 (R⁷=4-F-Phenyl, R¹⁰=3-MeO-Phenyl, R⁹=4-(4-Methyl-imidazol-1-yl), 33 mg, 0.06 mmol.) in THF (1 mL). The mixture was stirred at room temperature for 1 hour before it was diluted with ethyl acetate and washed with 0.5 N HCl followed by brine. The organic layer dried over anhydrous MgSO4 and concentrated to give the crude product which was purified using prep TLC eluting with ethyl acetate/methanol (10:1) to give 6 mg of AE3 (R⁷=4-F-Phenyl, R¹⁰=3-MeO-Phenyl, R⁹=4-(4-Methyl-imidazol-1-yl)).

¹H NMR (CDCl₃) δ: 7.61-7.57 (m, 2 H); 7.25 (m, 2 H); 7.20 (m, 2H); 7.10 (m, 2H); 6.95 (s, 1 H); 3.88 (s, 3H); 3.80 (AB quart, J=5.6, 6.4 Hz, 1 H); 3.10 (m, 2H); 2.30 (s, 3H); 1.95 (s, 3H); 1.65 (br. S, 1 H); 1.12 (d, J=6.4 Hz, 3 H). Electrospray LCMS: Obs. Mass: 449.2.

### Method AE, Step 3.

NaH (6 mg, 0.15 mmol.) was added to a solution of compound AE3 (R⁷=4-F-Phenyl, R¹⁰=3-MeO-Phenyl, R⁹=4-(4-Methyl-imidazol-1-yl)) (6 mg, 0.013 mmol.) in DMF (1.5 mL). The reaction solution was stirred at room temperature for one hour before it was quenched with water. The organic matter was extracted with ethyl acetate and the combined organic layer was washed with water, brine and dried over anhydrous sodium sulfate and concentrated. The residue was purified using prep TLC eluting with hexane/triethylamine (99:1) to obtain products AE4 (R⁷=4-F-Phenyl, R¹⁰=3-MeO-Phenyl, R⁹=4-(4-Methyl-imidazol-1-yl)). ¹H NMR (CDCl₃, ppm): 7.84 (s, 1 H), 7.70 (s, 1 H), 7.50 (m, 2H), 7.18 (s, 2H), 7.08 (t, 2H, J=8.8 Hz, 8.8 Hz), 6.23 (s, 1 H), 6.52 (s, 1 H), 4.12 (m, 1 H), 3.88 (s, 3H), 3.10 (dd, 1 H, J=3.2 Hz, 3.2 Hz), 2.73 (dd, 1 H, J=9.2 Hz, 8.8 Hz), 2.29 (s, 3H), 1.90 (s, 3H), 1.33 (d, 3H, J=6.0 Hz). MS (ES-LCMS, M+1) 449.

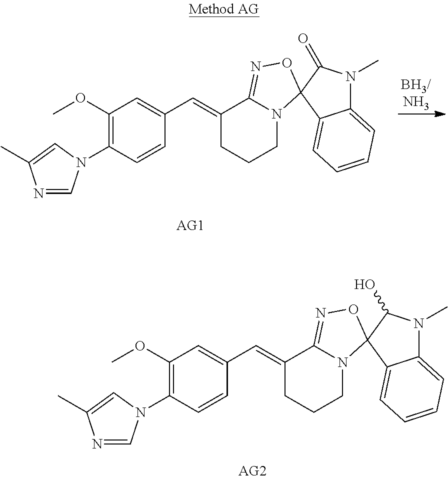

To a suspension of borane-ammonia complex (8 mg, 0.264 mmol) in THF (1 mL) was added n-BuLi 2.6 N in hexanes (0.10 ml, 0.264 mmol) at 0 C. The resulting solution was stirred at 0 C for 5 min, then at RT for 5 min. A solution of AG1 (40 mg, 0.088 mmol) in THF (1 mL) was then added at −78 C slowly and the reaction was stirred 1 h at this temperature. After quenching with MeOH and concentration, the crude was purified over silica gel (eluted with 0 to 10% MeOH in DCM) to give compound AG2. δ ¹H NMR (CDCl₃ 400 MHz): δ 7.72 (s, 1 H), 7.51 (s, 1 H), 7.45 (s, 1 H) 7.20-7.37 (m, 3H), 6.93-7.03 (m, 3H), 6.79-6.85 (m, 1 H), 6.56-6.61 (m, 1 H), 4.91 (s, 1 H), 4.74 (5, 1 H), 3.85 (s, 3H), 3.06-3.3 (m, 2H), 2.86 (s, 3H), 2.70 (m, 2H), 2.29 (s, 3H), 1.82-2.0 (m, 2H), LCMS (MH⁺)=458.3; retention time=2.89 min.

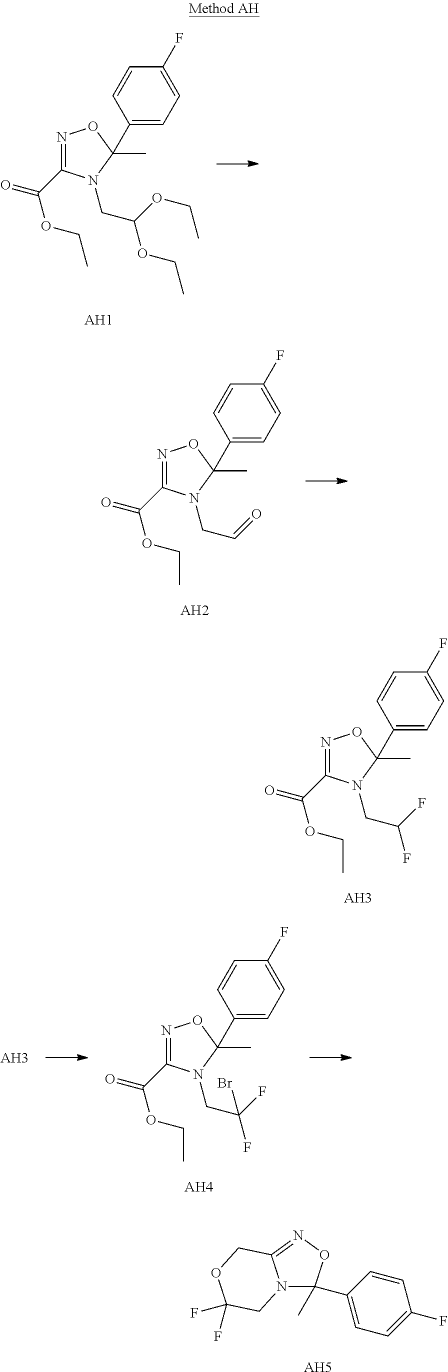

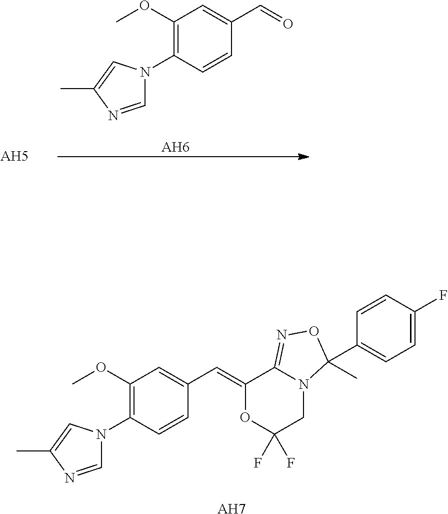

### Method AH, Step 1

Compound AH1 will be prepared using method similar to method A.

### Method AH, Step 2

Compound AH2 will be prepared treating AH1 with dilute HCl in acetone.

### Method AH, Step 3

Compound AH3 will be prepared by treating compound AH2 with DAST.

### Method AH, Step 4

Compound AH4 will be obtained by treating AH3 with NBS and a radical initiator such as AIBN under reflux CCl4

### Method AH, Step 5

Compound AH5 will be prepared by treating AH4 with NaBH₄

### Method AH, Step 6,

Compound AH7 will be prepared by treating AH5 with BuLi followed by AH6

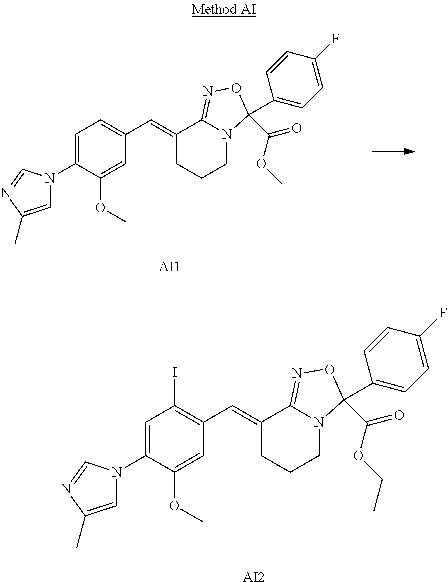

To a solution of AI1 (144 mg, 0.302 mmol, 1.0 equiv.) in 1.5 mL neat CF₃SO₃H was added NIS (135 mg, 0.605 mmol, 2 equiv.) at 0° C., and the resulting mixture was stirred for 15 minutes at this temperature. Then, the reaction mixture was poured into ice water containing sodium thiosulfate. The resulting mixture was extracted with ethyl acetate, dried with MgSO4, concentrated and purified using C18 Column (0.1% TFA in water and 0.1% TFA in acetonitrile was used as eluent) to yield AI2 in 70% yield. ¹H NMR: δ 8.02 (s, 1 H), 7.73 (s, 1 H), 7.52-7.49 (m, 2H), 7.37 (s, 1 H), 7.12 (t, J=8.8 Hz, 2H), 6.9 (s, 1 H), 6.88 (s, 1 H), 4.34 (m, 2H), 3.8 (s, 3H), 3.60 (m, 1 H), 3.13 (m, 1 H), 2.85 (m, 1 H), 2.50 (m, 2H), 2.33 (s, 3H), 1.93 (m, 1 H), 1.76 (m, 1 H), 1.33 (t, J=6.9 Hz, 3H).

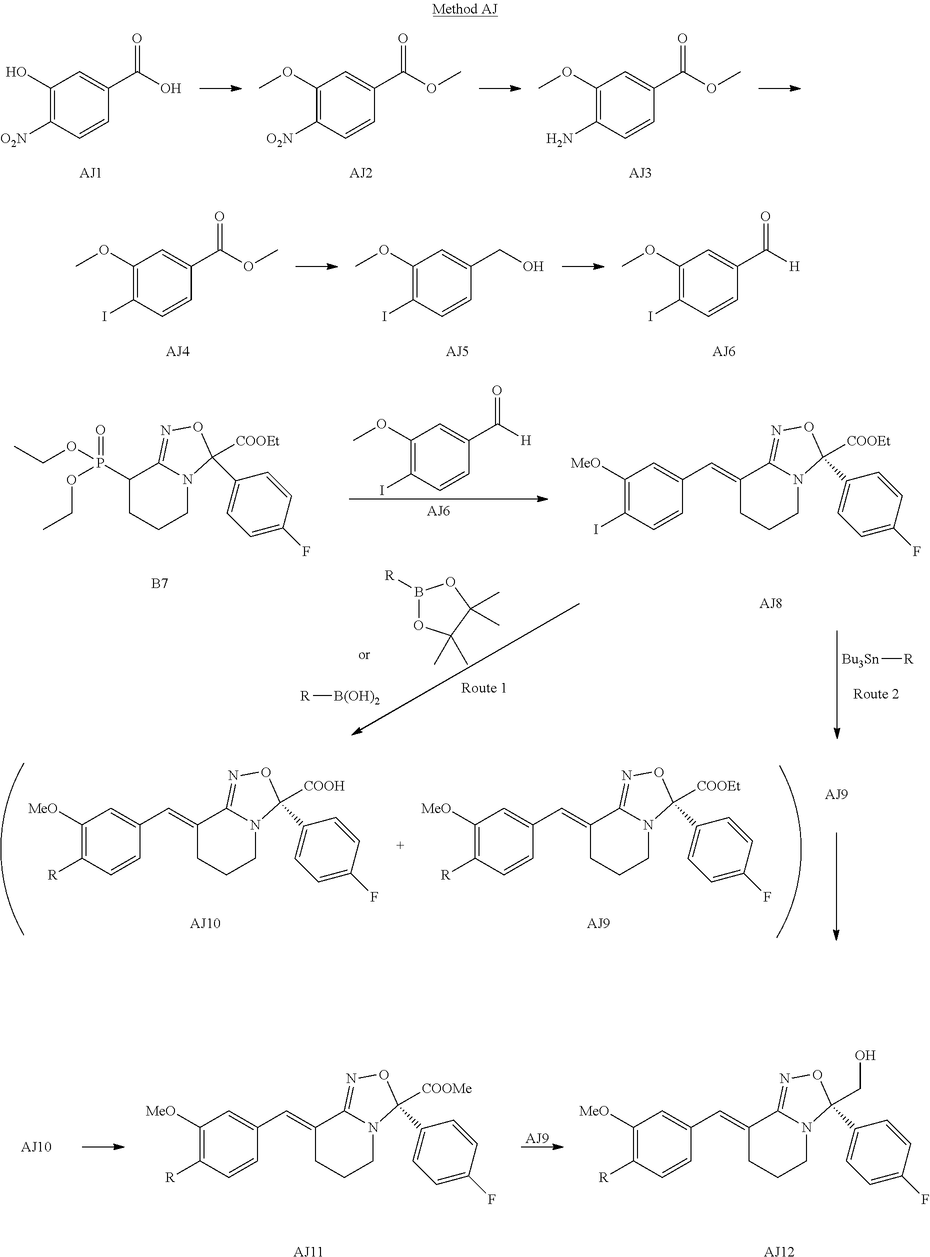

### Step 1, AJ2

To a 12 L 3-necked round bottomed flask equipped with an addition funnel, under nitrogen and containing a solution of AJ1 (302.7 g, 1.65 mol) in DMF (2.5 L) was added K₂CO₃ (905.3 g, 6.55 mol) portionwise over 5 min. Methyl iodide was then added dropwise via addition funnel over 70 min. and then the mixture was stirred overnight. The reaction mixture was slowly poured into an XL extractor containing a stirring mixture of water (7 L) and ice (3 L). The resulting mixture was extracted with ethyl acetate (1×6 L, 1×4 L), washed with water (1×4 L), and brine (1×2 L). The combined organic layers were dried over MgSO₄, filtered, and concentrated in vacuo to afford AJ2 (344 g, 97%) as yellow needles. ¹HNMR (CDCl₃, 400 MHz) δ 7.80 (d, 1 H), 7.73 (d, 1 H), 7.66 (dd, 1 H), 3.99 (s, 3H), 3.94 (s, 3H).

### Step 2, AJ3

To a 2 L Parr bottle containing a mixture of AJ2 (95 g, 0.45 mol) in MeOH (anhydrous, 1.3 L) under nitrogen was added (Raney nickel slurry in water (15 ml) exchanged with methanol 3 times). The reaction mixture was hydrogenated in a Parr shaker at 45 psi overnight. The reaction sat for 30 min. The top layer of the reaction mixture was decanted and filtered. The residue was diluted with DCM (1 L), swirled for 5 min., and filtered resulting in AJ3 (> quantitative) as an off-white solid. ¹HNMR (CDCl₃, 400 MHz) δ 7.52 (dd, 1 H), 7.43 (d, 1 H), 6.63 (d, 1 H), 4.21 (s, 2H), 3.88 (s, 3H), 3.84 (s, 3H).

### Step 3, AJ4

To a 12 L 3-necked round bottomed flask equipped with a mechanical stirrer, thermometer, addition funnel, nitrogen inlet, and containing a suspension of AJ3 (252 g, 1.39 mol) in water (3.5 L) at 0° C. was added H₂SO₄ (20% vol., 700 mL). A solution of NaNO₂ (105.6 g, 1.53 mol) in water (550 mL) was added slowly over 1 h at 0° C. to 3° C. and the reaction mixture was stirred further for 1 h. Next, urea (25 g, 0.417 mol) was added to the reaction mixture portionwise and stirred for 15 min. Then a solution of KI (242.3 g, 1.46 mol) in water (600 mL) was added to the 0° C. reaction mixture over 30 min. The reaction mixture was then heated at 55° C. for 1.5 h. Next, ethyl acetate (4 L) was used to dissolve the reaction mixture and the resulting solution was poured slowly into a solution of Na₂S₂O₅ (650 g) in ice water (4 L) and the flask was rinsed with ethyl acetate (2 L) and stirred for 15 min. The resulting layers were separated and the aqueous phase (pH-3) was extracted with ethyl acetate (2 L). The combined organic layers were washed with water (2 L×2), brine (1 L), dried over Mg₂SO₄, filtered, and concentrated in vacuo. The crude material was purified via silica gel plug (ethyl acetate/hexanes) to afford AJ4 (370 g, 91%) as a white solid. ¹HNMR (CDCl₃, 400 MHz) δ 7.83 (d, 1 H), 7.43 (d, 1 H), 7.35 (dd, 1 H), 3.93 (s, 3H), 3.90 (s, 3H).

### Step 4, AJ5

To a 12 L 3-necked round bottomed flask equipped with a mechanical stirrer, thermometer, nitrogen inlet, and containing a solution of AJ4 (270 g, 0.925 mol) in THF (4 L) was added LiBH₄ (60.4 g, 2.77 mol) portionwise at room temperature. The reaction mixture was placed in an ice bath and methanol (135 mL) was added dropwise. After the addition was complete the ice bath was removed and the reaction was heated to 65° C. for 1 h. The reaction was then cooled in an ice bath and poured into an ice cold solution of saturated aq. NH₄Cl (2 L) and ethyl acetate (4 L) followed by rinsing of the flask with ethyl acetate (2 L). The solution was stirred for 15 min., the layers were separated and the aqueous layer was extracted with ethyl acetate (4 L). The combined organic layers were washed with water (2 L×2), brine (1 L), dried over MgSO₄, filtered and concentrated in vacuo to afford AJ5 (> quantitative) as a light-yellow oil. ¹HNMR (CDCl₃, 400 MHz) δ 7.70 (d, 1 H), 6.86 (d, 1 H), 6.67 (dd, 1 H), 4.64 (d, 2H), 3.88 (s, 3H).

### Step 5, AJ6

To a 12 L 3-necked round bottomed flask equipped with a mechanical stirrer, thermometer, addition funnel, nitrogen inlet, and containing a solution of (COCl)₂ (123.7 g, 0.975 mol) in DCM (3.5 L) at −70° C. was added a solution of DMSO (173 g, 2.215 mol) in DCM (250 mL) over 30 min. and was stirred an additional 30 min. at −72° C. Next, a solution of AJ5 (234 g, 0.886 mol) in DCM (1 L) was added over 1.5 h to the reaction solution keeping the reaction temperature between −65° C. and −70° C. and then the reaction solution was stirred for an additional 30 min. at −70° C. Next, triethylamine (363 g, 3.587 mol) was added over 15 min. and then the reaction mixture was stirred for an additional 1 h at −65° C. The cooling bath was removed and the reaction mixture was poured into an extractor filled with ice water (3 L) and stirred for 15 min. The layers were separated and the aqueous layer was extracted with DCM (2 L). The combined organic layers were washed with HCl (1 N, 1.5 L), water (2 L×3), brine (1 L), dried over MgSO₄, filtered, and dried in vacuo. The crude material was triturated with hexanes (300 mL), filtered, washed with hexanes (100 mL×2), and dried under vacuum to afford AJ6 (212.7 g, 92%) as an off-white solid. ¹HNMR (CDCl₃, 400 MHz) δ 9.93 (s, 1 H), 7.96 (d, 1 H), 7.27 (d, 1 H), 7.17 (dd, 1 H), 3.94 (s, 3H).

### Step 7, AJ8

To a vacuum dried round bottomed flask, equipped with an addition funnel, under nitrogen, and containing the phosphonate AJ7 (1.72 g, 4.02 mmol) was added a solution of the aldehyde AJ6 (1.0 g, 3.82 mmol) in THF (24 mL). The reaction vessel was then cooled to −78° C. In a separate flask, t-BuOK (0.495 g, 4.42 mmol) was dried under vacuum, placed under nitrogen, and dissolved in THF (16 mL). The t-BuOK solution was transferred to the addition funnel and was added dropwise to the phosphonate flask at −78° C. After the reaction was warmed to −30° C. over 4 h, t-BuOK (0.045 g, 0.4 mmol) was added. After 1 hr at −30° C., t-BuOK (0.045 g, 0.4 mmol) was added. After an additional 1 hr at −30° C., the reaction was poured over a 0° C. mixture of brine and saturated aq. NH₄Cl. The resulting mixture was then extracted with ethyl acetate. The combined organic layers were washed with brine, dried over Na₂SO₄, and concentrated in vacuo. The crude material was purified by silica gel chromatography with methanol/DCM. This material was then resolved by chiral AS column with isopropyl alcohol/hexanes to afford compound AJ8 (0.434 g, 42%) as a yellow foam. ¹HNMR (CDCl₃, 400 MHz) δ 7.73 (d, 1 H), 7.48 (m, 3H), 7.10 (t, 2H), 6.72 (d, 1 H), 6.67 (d, 1 H), 4.32 (m, 2H), 3.86 (s, 3H), 3.55 (m, 1 H), 2.85 (m, 1 H), 2.65 (m, 2H), 1.92 (m, 1 H), 1.72 (m, 1 H), 1.33 (t, 3H); MS (LCMS, M+1) 537.3.

### Route 1

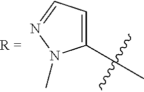

Step 1: To a microwave vial under nitrogen was added compound AJ8 (0.075 g, 0.139 mmol), Pd(PPh₃)₄ (0.016 g, 0.0139 mmol) 1-methyl-1 H-pyrazole-5-boronic acid pinacol ester (0.091 g, 0.417 mmol), Na₂CO₃ (0.044 g, 0.417 mmol) in water (0.5 mL), and acetonitrile (2.5 mL). This mixture was then heated in a microwave to 130° C. for 30 min. on high absorption. The resulting mixture was then poured over iced-brine, and then extracted with ethyl acetate. The combined organic layers were dried over Na₂SO₄, and concentrated in vacuo. The crude material was purified by silica gel chromatography with methanol/ammonium hydroxide/DCM to afford crude compounds AJ9 (MS (LCMS, M+1) 491.2) and AJ10 (MS (LCMS, M+1) 477.2).
Alternative Method for Step 1: To a round bottomed flask under nitrogen was added compound AJ8 (1 equiv.), boronic ester/acid (1.2 equiv.), Pd(PPh₃)₄ (0.06 equiv.), Na₂CO₃ (2.4 equiv.), toluene, ethanol, and water. The reaction mixture was heated to 100° C. overnight.
Alternative Method for Step 1: To a round bottomed flask under nitrogen was added compound AJ8 (1 equiv.), boronic ester/acid (4 equiv.), Pd(C1)₂dppf (0.1 equiv.), K₃PO₄ (10 equiv.), and dioxane. The reaction mixture was heated to 85° C. overnight.

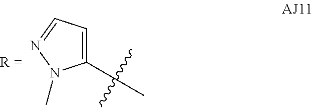

Step 2: To a vial under nitrogen containing a mixture of the crude acid AJ10 (0.188 g) in DCM/methanol (2 mL/1 mL) was added TMS-diazomethane (2 M, 2 mL). The vial was capped and the reaction mixture stirred for 1.5 h. The reaction mixture was then concentrated in vacuo and purified by silica gel chromatography with methanol/ammonium hydroxide/DCM to afford crude compound AJ11. MS (LCMS, M+1) 477.2.

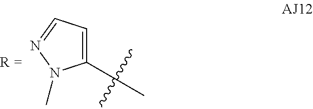

Step 3: To a solution of crude ester 9 (0.020 g) and crude ester AJ11 (0.022 g) in methanol/ethanol (0.5 mL/1 mL) at 0° C. was added NaBH₄ (0.003 g). The mixture was removed from the ice-bath after 20 min and stirred for an additional 40 min. The reaction mixture was again cooled to 0° C. and NaBH₄ (0.003 g) was added. The mixture was removed from the ice-bath after 20 min and stirred for an additional 50 min. The reaction mixture was again cooled to 0° C. and NaBH₄ (0.010 g) was added. After 1 hr the reaction mixture was poured over iced-brine, and then extracted with ethyl acetate. The combined organic layers were dried over Na₂SO₄, and concentrated in vacuo. The crude material was purified by silica gel chromatography with methanol/ammonium hydroxide/DCM to afford compound AJ12 (0.042 g, 42% over steps 1-3). ¹HNMR (CDCl₃, 400 MHz) δ 7.50 (m, 4H), 7.22 (d, 1 H), 7.09 (t, 2H), 6.97 (d, 1 H), 6.89 (s, 1 H), 6.23 (d, 1 H), 4.18 (d, 1 H), 4.03 (d, 1 H), 3.79 (s, 3H), 3.73 (s, 3H), 3.32 (m, 1 H), 2.97 (m, 1 H), 2.73 (m, 2H), 2.39 (s, 1 H), 1.92 (m, 2H); MS (LCMS, M+1) 449.2.

### Route 2

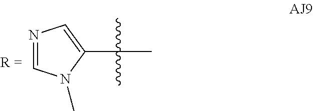

To a 2 mL microwave vial under nitrogen was added AJ8 (0.100 g, 0.186 mmol), 1-methyl-5-(tributylstannyl) imidazole (0.138 g, 0.373 mmol), Pd(PPh₃)₄ (0.016 g, 0.0139 mmol), i-Pr₂NEt (0.097 mL, 0.558 mmol), THF (0.93 mL), and PhCF₃ (0.31 mL). The reaction mixture was heated in a microwave vial at 150° C. for 45 min. under normal absorption. The resulting mixture was poured over iced-brine, and then extracted with ethyl acetate. The combined organic layers were dried over Na₂SO₄, and concentrated in vacuo. The crude material was purified via silica chromatography (methanol/NH₄OH/DCM) to afford crude compound AJ9 as a yellow film. MS (LCMS, M+1) 491.2.

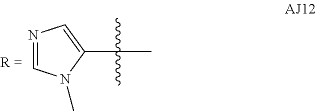

Compound AJ12 was synthesized as described in Route 1 Step 3. MS (LCMS, M+1) 449.4.

The following compounds were synthesized using a method similar to the method listed in the last column.

| | | | Obs | Synthetic |
|---|---|---|---|---|
| # | Structure | Rt (min) | Mass | Method |
| 201 | 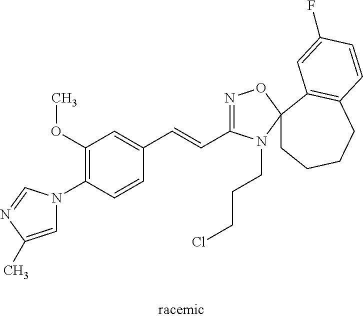 | 3.4 | 509 | A |
| 202 | 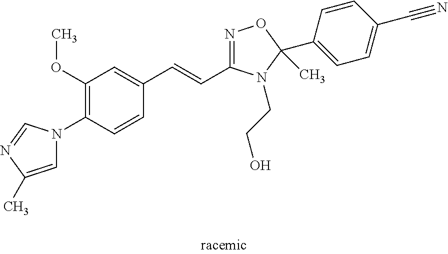 | 2.6 | 444 | A |
| 203 | 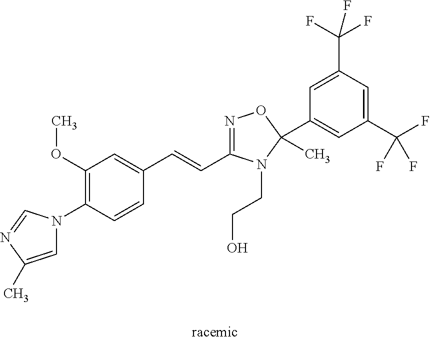 | 3.3 | 555 | A |
| 204 | 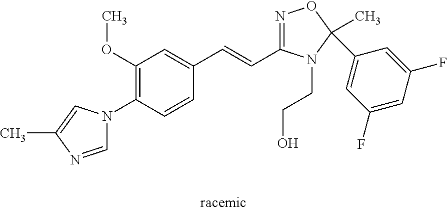 | 1.9 | 455 | A |
| 205 | 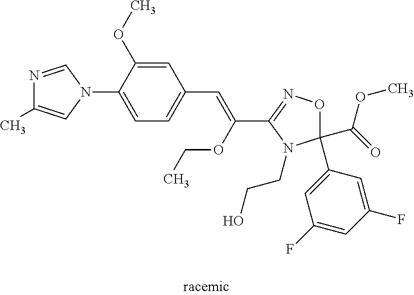 | 3.3 | 543 | A |
| 206 | 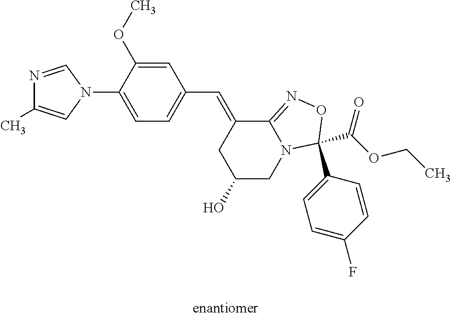 | 3 | 507 | AA |
| 207 | 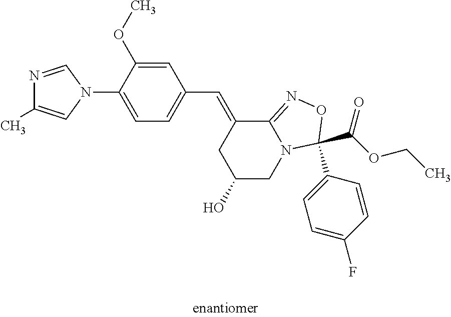 | 3 | 507 | AA |
| 208 | 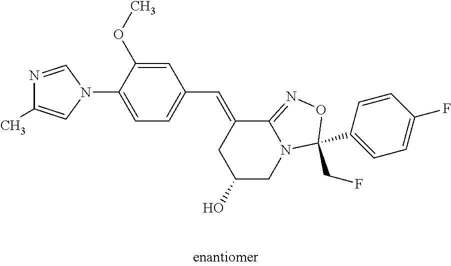 | 2.4 | 465 | AA |
| 209 | 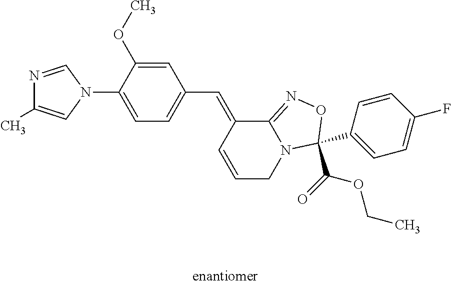 | 3.3 | 489 | AB |
| 210 | 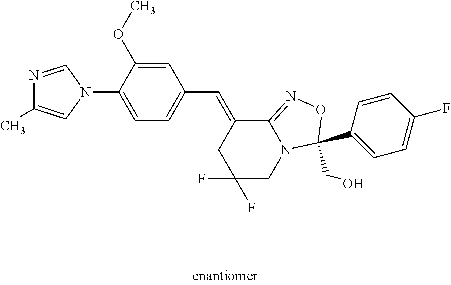 | 3.05 | 485 | AC |
| 211 | 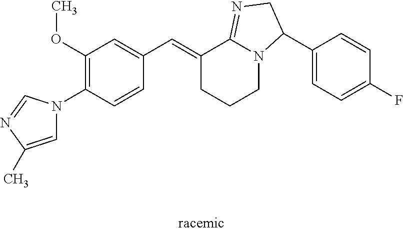 | 2.3 | 417 | AD |
| 212 | 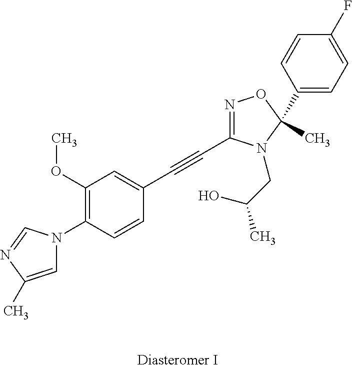 | 2.7 | 449 | AE |
| 213 | 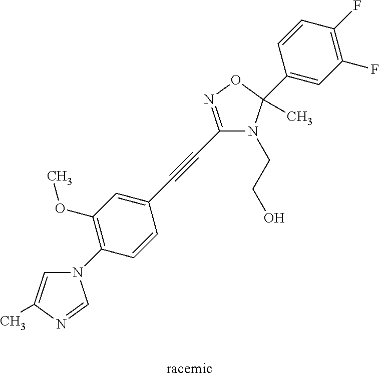 | 2.9 | 453 | AE |
| 214 | 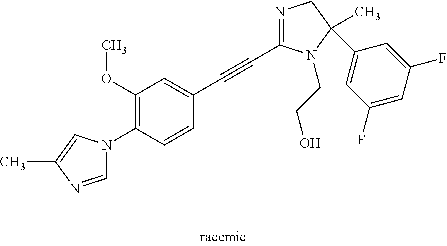 | 3 | 453 | AE |
| 216 | 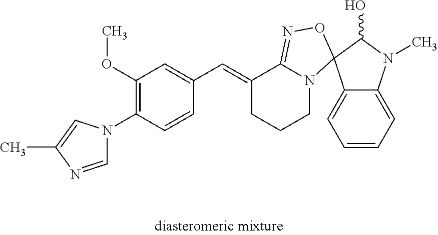 | 2.9 | 458 | AG |
| 217 | 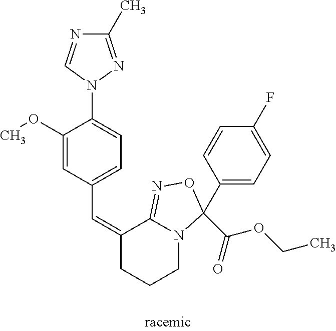 | 4.5 | 492 | B |
| 218 | 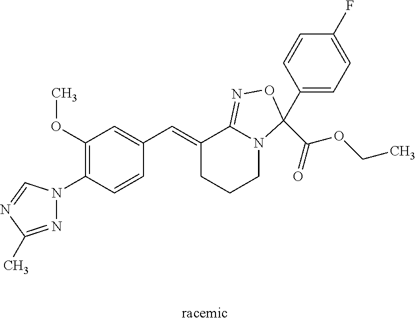 | 4.5 | 492 | B |
| 219 | 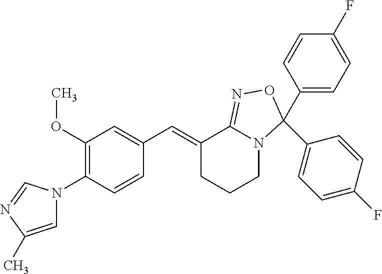 | 3.9 | 513 | B |
| 220 | 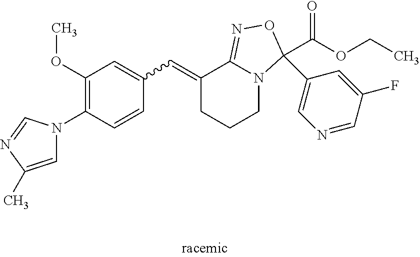 | 2.83 | 492 | B |
| 221 | 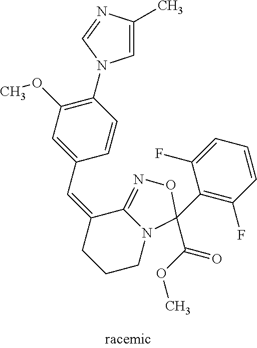 | 3 | 495 | B |
| 222 | 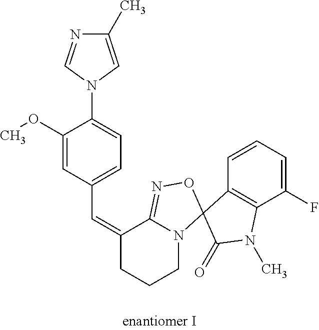 | 3.2 | 474 | B |
| 223 | 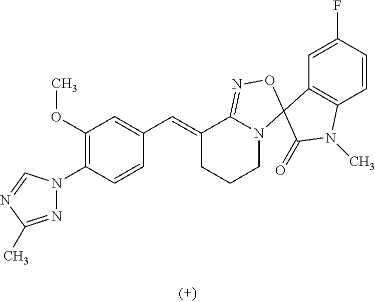 | 3.7 | 474 | B |
| 224 | 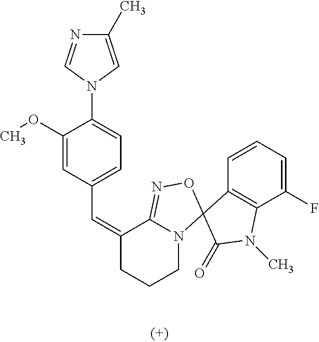 | 3.1 | 474 | B |
| 225 | 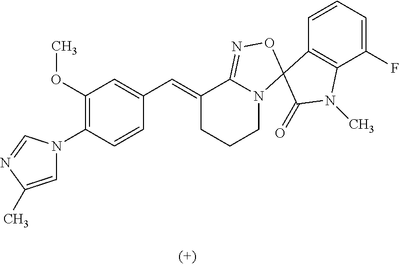 | 3.1 | 474 | B |
| 226 | 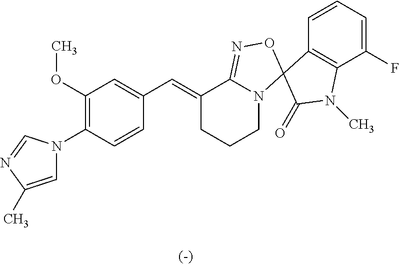 | 3.1 | 474 | B |
| 227 | 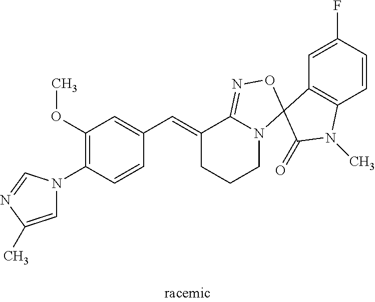 | 3.8 | 474 | B |
| 228 | 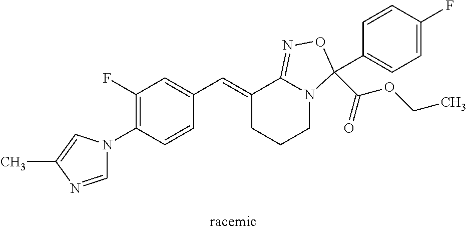 | 3.13 | 479 | B |
| 229 | 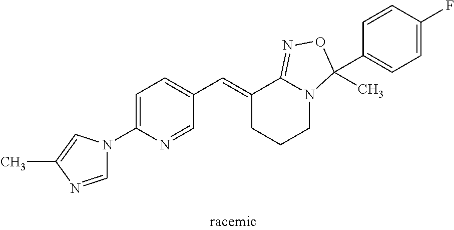 | 3.17 | 404 | B |
| 230 | 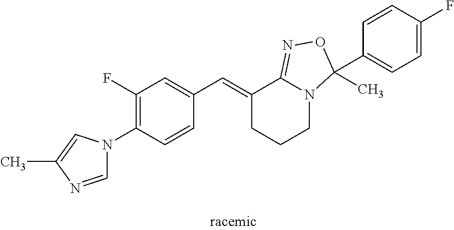 | 3.04 | 421 | B |
| 231 | 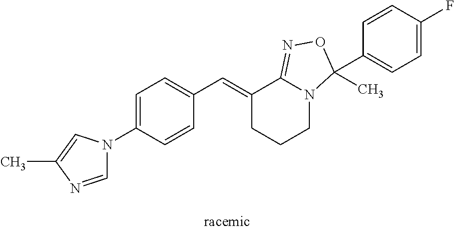 | 3 | 403 | B |
| 232 | 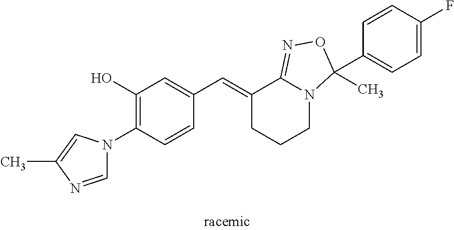 | | | B |
| 233 | 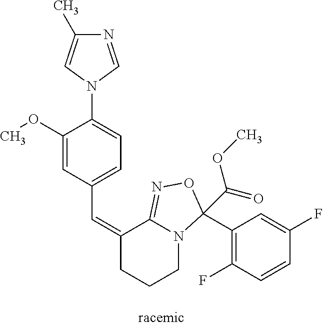 | 3.2 | 495 | B |
| 234 | 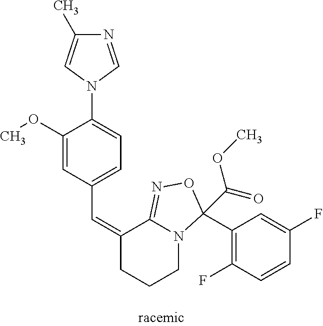 | 3.3 | 495 | B |
| 235 | 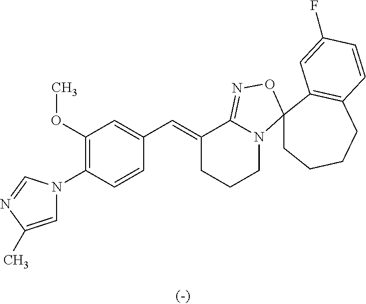 | 3.4 | 473 | B |
| 236 | 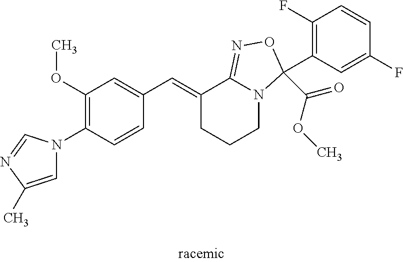 | 3.2 | 495 | B |
| 237 | 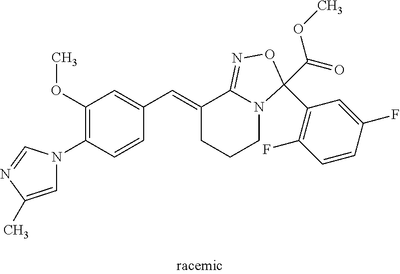 | 3.3 | 495 | B |
| 238 | 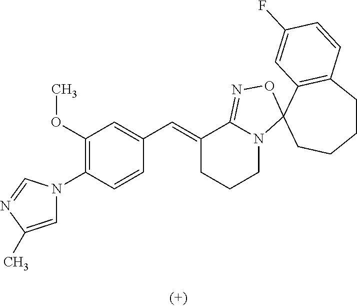 | 3.4 | 473 | B |
| 239 | 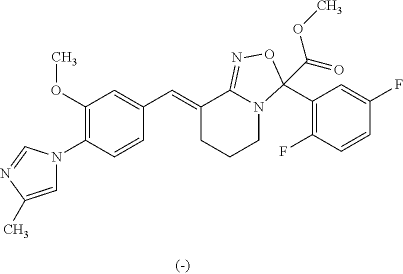 | 3.3 | 495 | B |
| 240 | 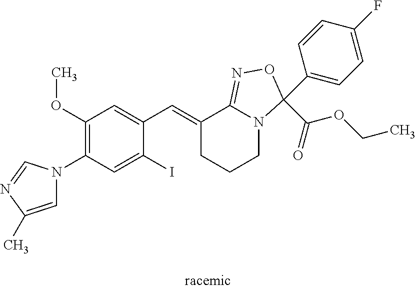 | 3.5 | 617 | B |
| 241 | 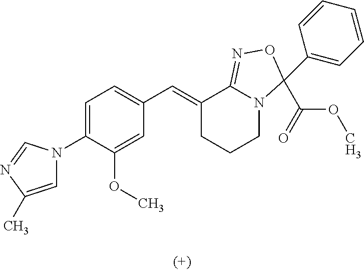 | 1.975(A) | 459 | B |
| 242 | 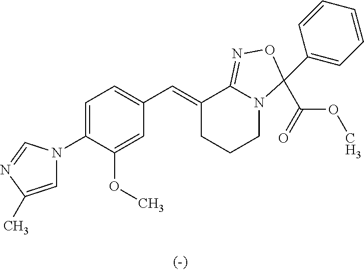 | 1.98(A) | 459 | B |
| 243 | 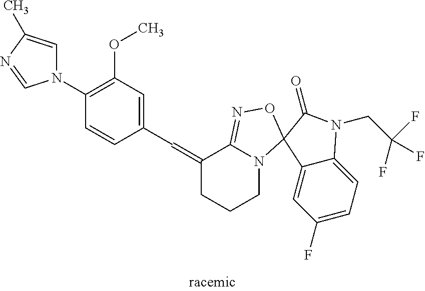 | 3.1 | 542 | B |
| 244 | 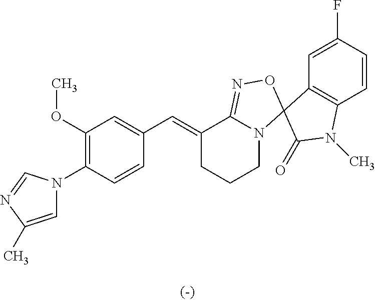 | 3 | 474 | B |
| 245 | 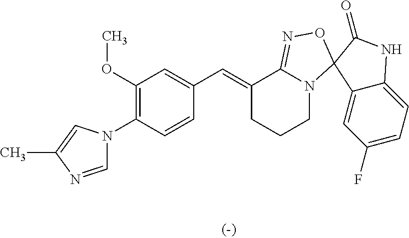 | 2.6 | 460 | B |
| 246 | 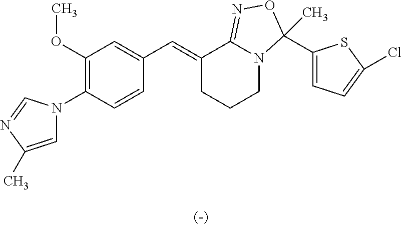 | | | B |
| 247 | 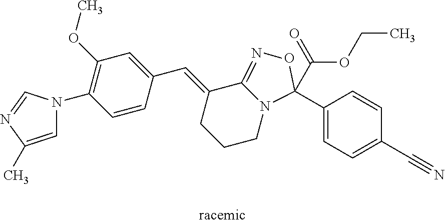 | 3.1 | 498 | B |
| 248 | 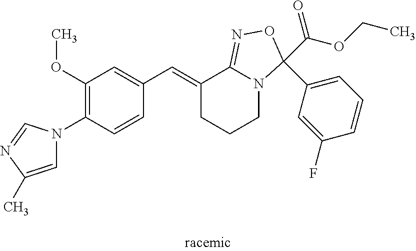 | 3.2 | 491 | B |
| 249 | 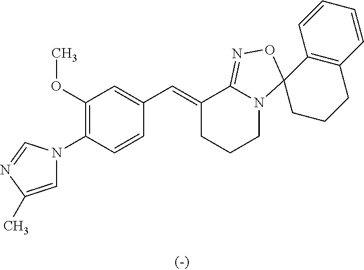 | 3.7 | 441 | B |
| 250 | 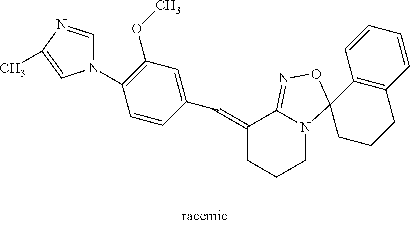 | 3.1 | 441 | B |
| 251 | 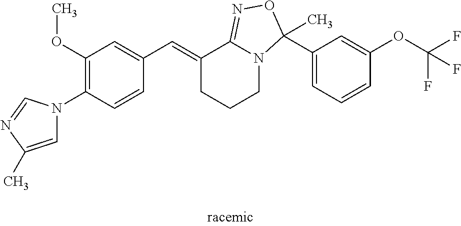 | 3.4 | 499 | B |
| 252 | 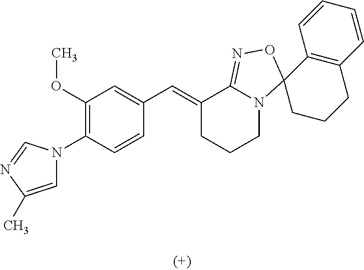 | 3.6 | 441 | B |
| 253 | 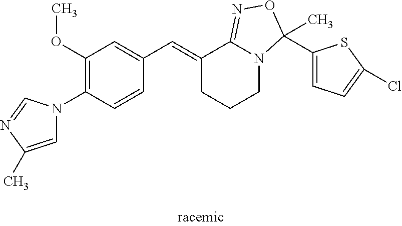 | 3.1 | 455 | B |
| 254 | 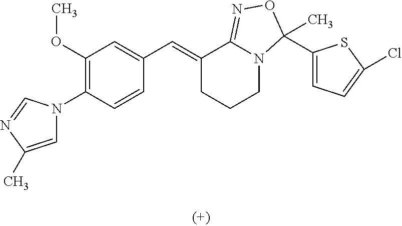 | | | B |
| 255 | 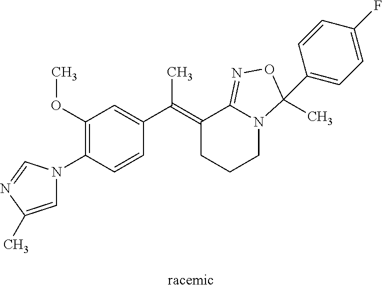 | 3.1 | 447 | B |
| 256 | 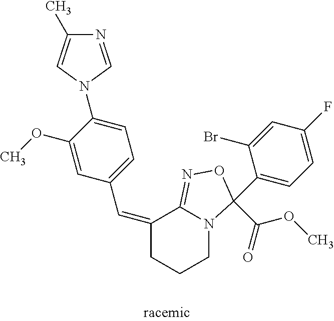 | 3.3 | 556 | B |
| 257 | 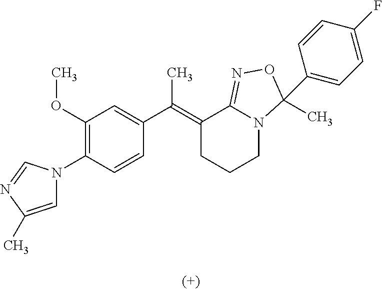 | 3.1 | 447 | B |
| 258 | 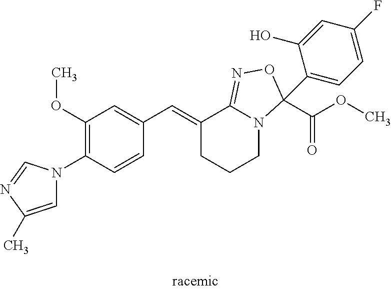 | 2.8 | 493 | B |
| 259 | 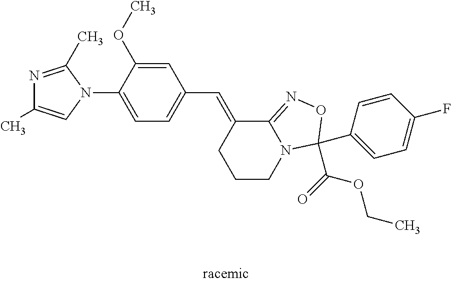 | | | B |
| 260 | 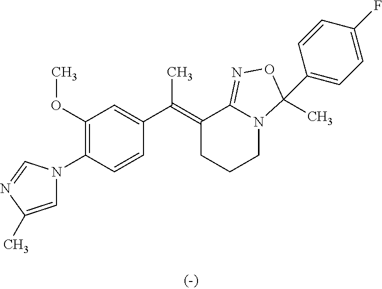 | 3.1 | 447 | B |
| 262 | 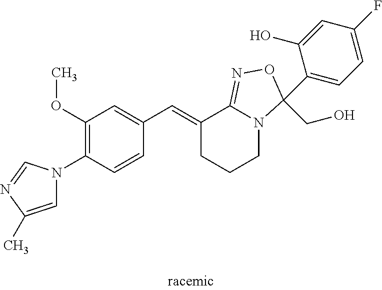 | 2.6 | 465 | B |
| 264 | 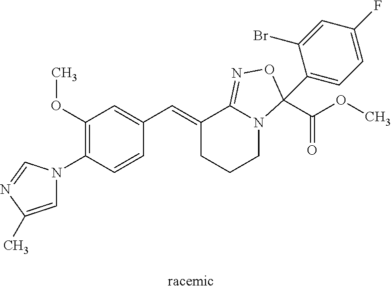 | 3.4 | 566 | B |
| 265 | 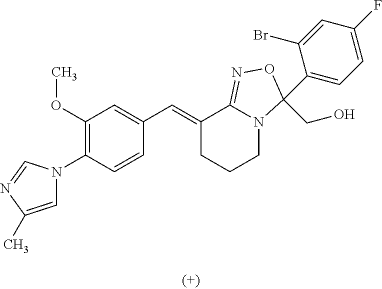 | 2.9 | 528 | B |
| 266 | 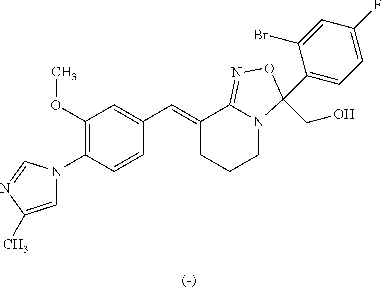 | 2.9 | 528 | B |
| 268 | 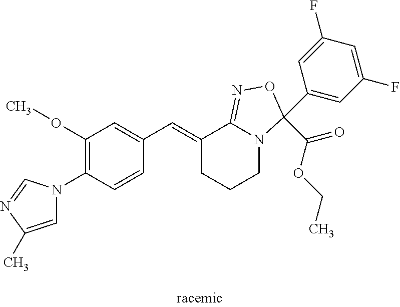 | 3.76(A) | 509 | B |
| 269 | 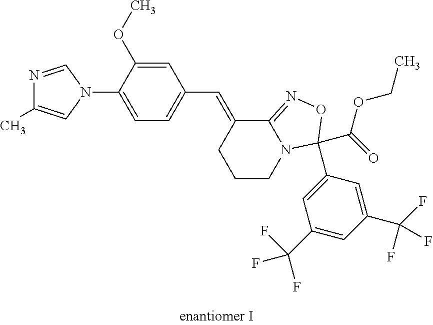 | 3.9 | 609 | B |
| 270 | 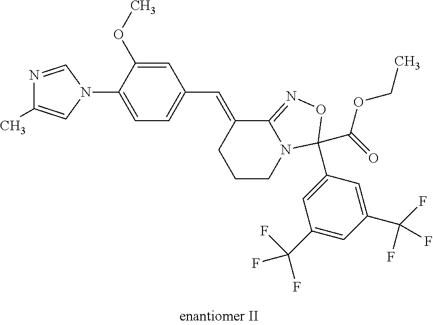 | 3.9 | 609 | B |
| 271 | 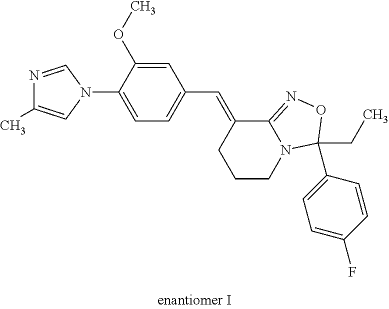 | 3.4 | 447 | B |
| 272 | 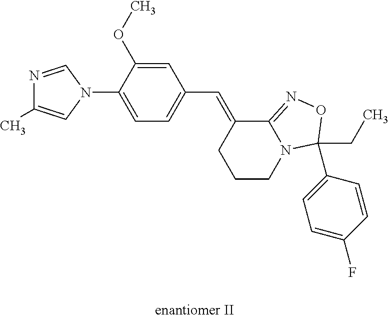 | 3.4 | 447 | B |
| 273 | 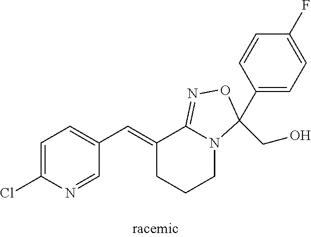 | | | C |
| 274 | 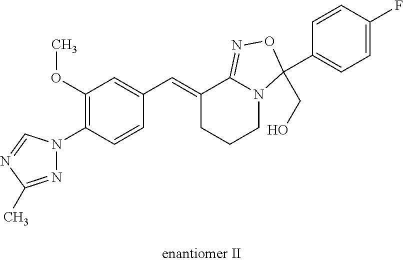 | 3.68 | 450 | C |
| 275 | 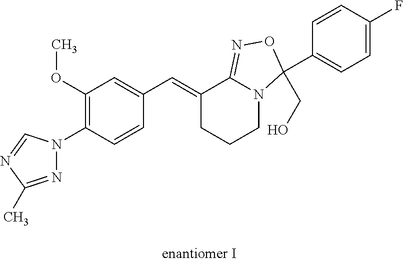 | 3.69 | 450 | C |
| 276 | 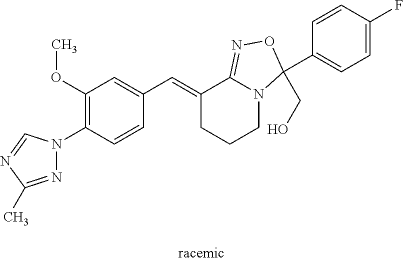 | 3.6 | 450 | C |
| 277 | 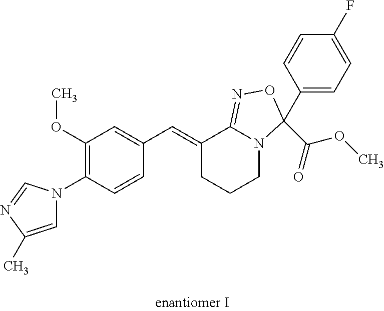 | 3.22 | 479 | C |
| 278 | 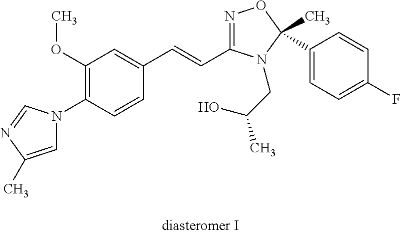 | 2.88 | 451 | C |
| 279 | 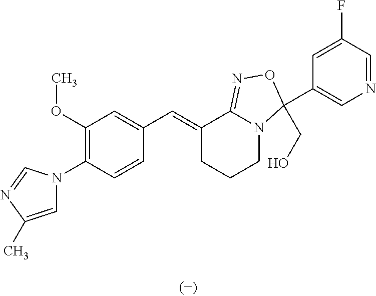 | 2.4 | 450 | C |
| 280 | 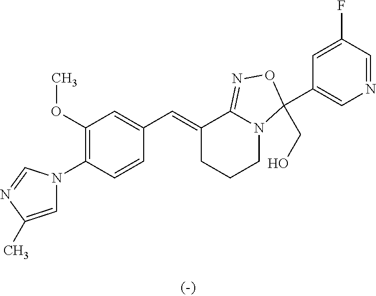 | 2.4 | 450 | C |
| 281 | 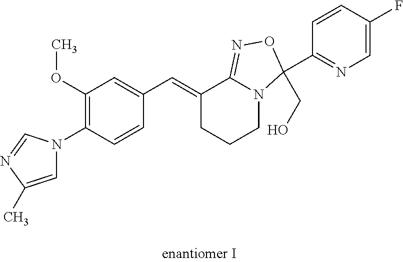 | 2.6 | 450 | C |
| 282 | 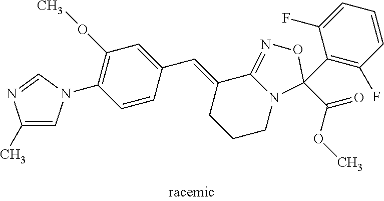 | 2.9 | 495 | C |
| 283 | 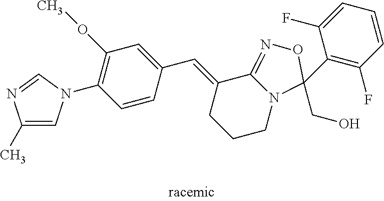 | 2.7 | 467 | C |
| 284 | 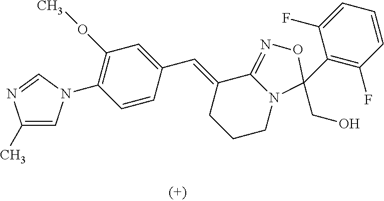 | 2.5 | 467 | C |
| 285 | 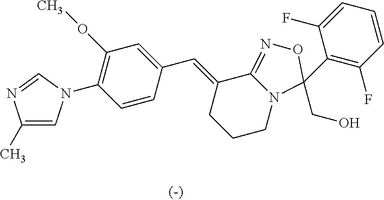 | 2.5 | 467 | C |
| 286 | 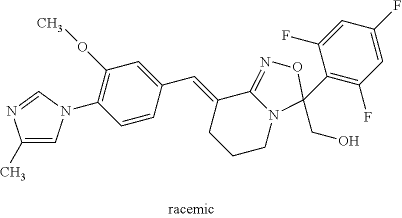 | 2.6 | 485 | C |
| 287 | 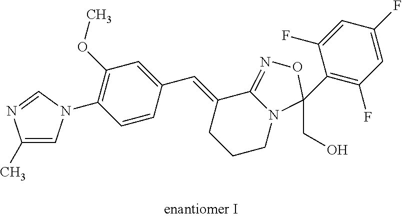 | 2.8 | 485 | C |
| 288 | 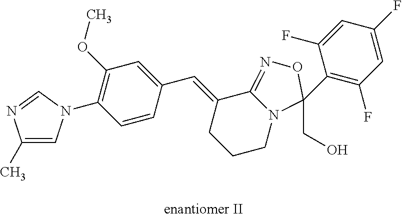 | 2.8 | 485 | C |
| 289 | 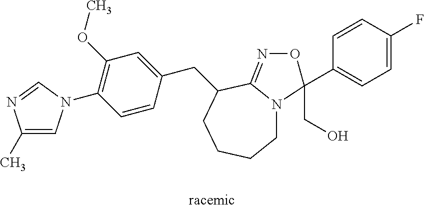 | 2.8 | 463 | C |
| 290 | 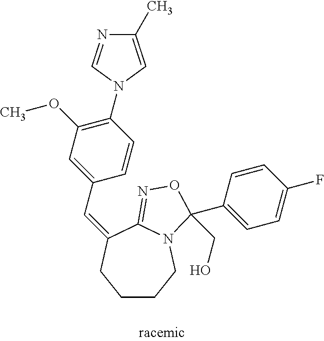 | 2.9 | 463 | C |
| 291 | 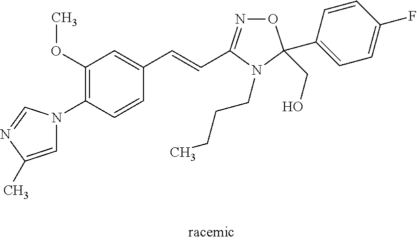 | 3.1 | 465 | C |
| 292 | 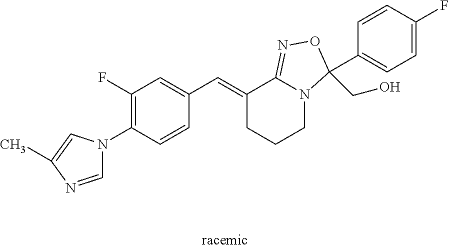 | 2.4 | 437 | C |
| 293 | 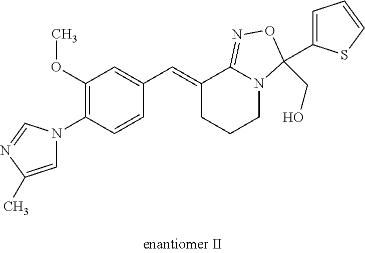 | 2.6 | 437 | C |
| 294 | 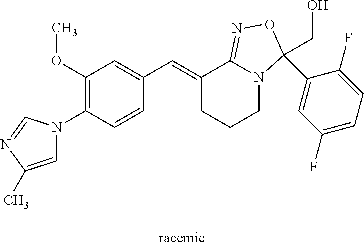 | 2.8 | 467 | C |
| 295 | 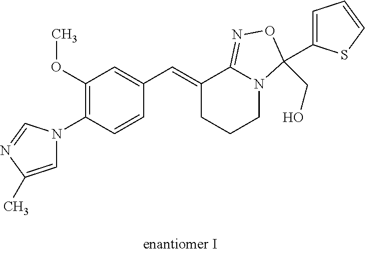 | 2.6 | 437 | C |
| 296 | 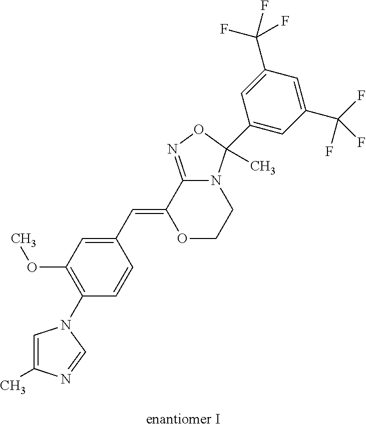 | 3.5 | 553 | C |
| 297 | 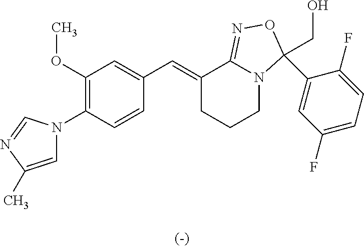 | 2.8 | 487 | C |
| 298 | 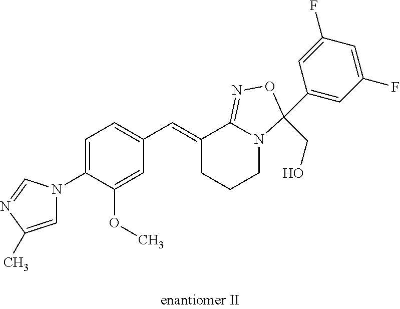 | 2.9 | 467 | C |
| 299 | 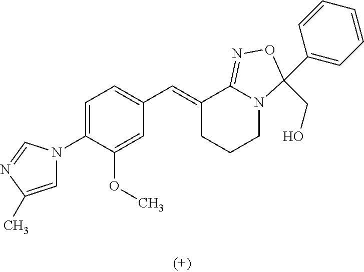 | 1.85(A) | 431 | C |
| 300 | 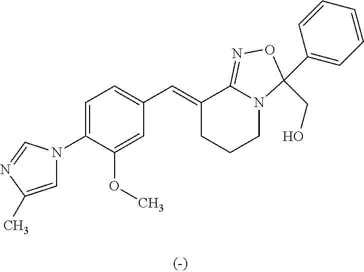 | 1.86(A) | 431 | C |
| 301 | 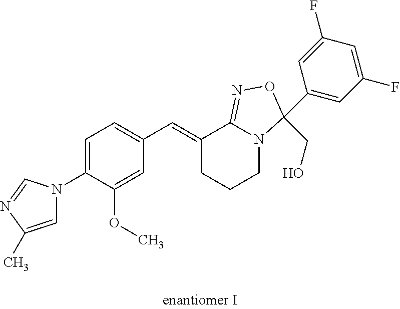 | 2.8 | 467 | C |
| 302 | 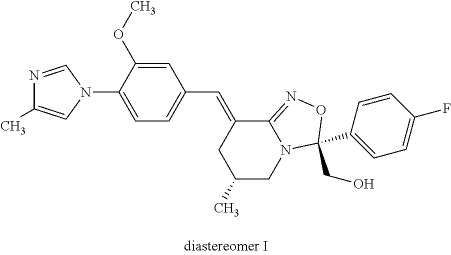 | 2.9 | 463 | C |
| 303 | 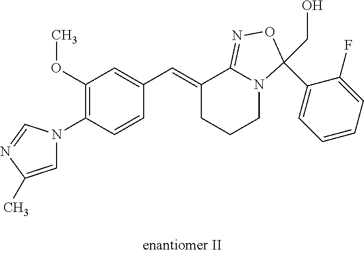 | 2.6 | 449 | C |
| 304 | 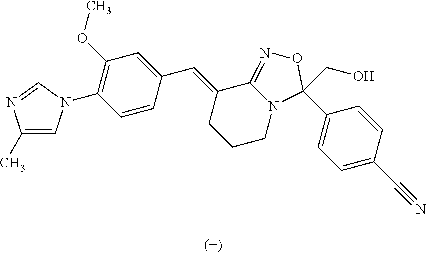 | 2.6 | 456 | C |
| 305 | 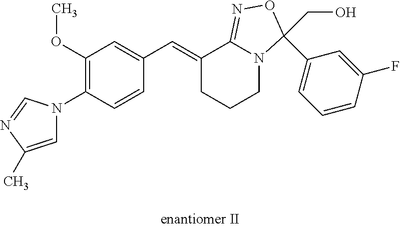 | 2.8 | 449 | C |
| 306 | 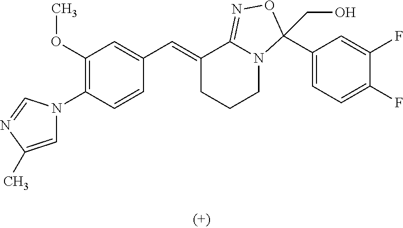 | | | C |
| 307 | 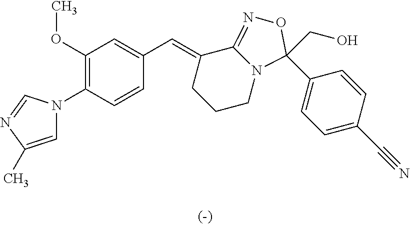 | 2.6 | 456 | C |
| 308 | 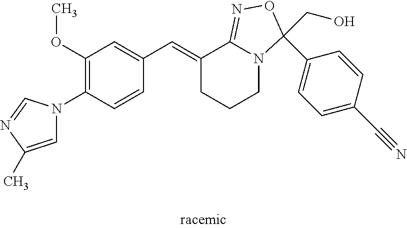 | 2.4 | 456 | C |
| 309 | 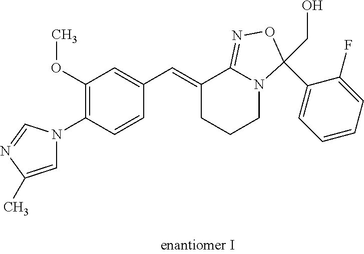 | 2.6 | 449 | C |
| 310 | 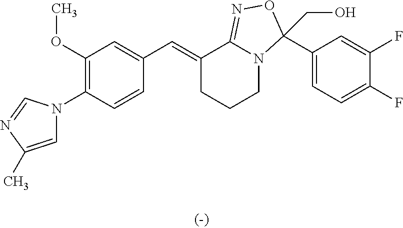 | 3 | 467 | C |
| 311 | 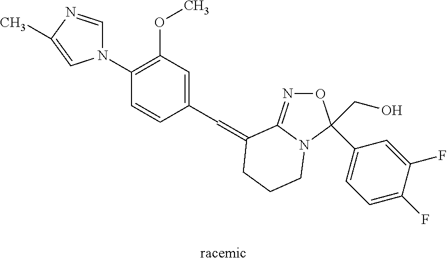 | 2.6 | 467 | C |
| 312 | 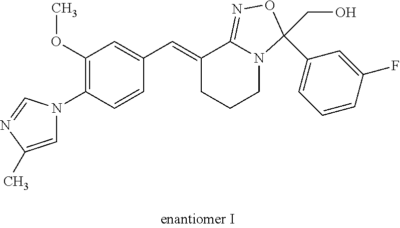 | 2.7 | 449 | C |
| 313 | 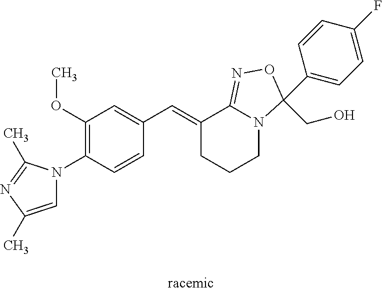 | | | C |
| 314 | 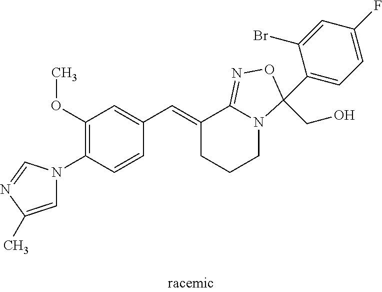 | 2.9 | 528 | C |
| 315 | 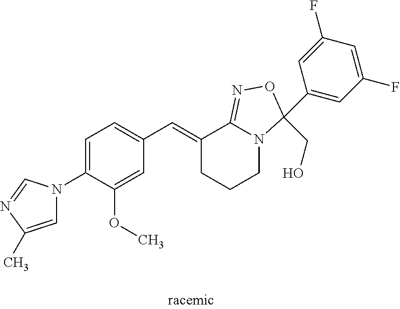 | 3.2 | 467 | C |
| 316 | 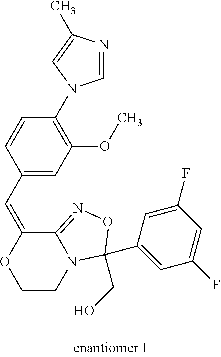 | 2.9 | 469 | C |
| 317 | 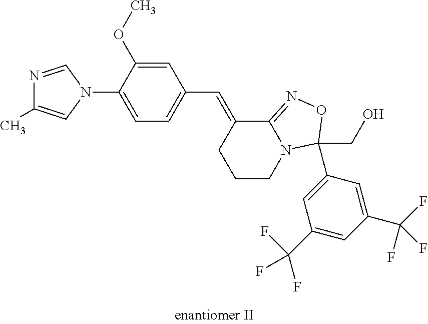 | 3.2 | 567 | C |
| 318 | 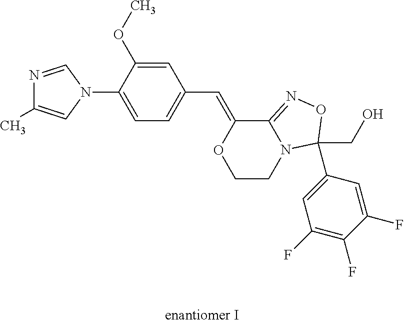 | 3.1 | 487 | C |
| 319 | 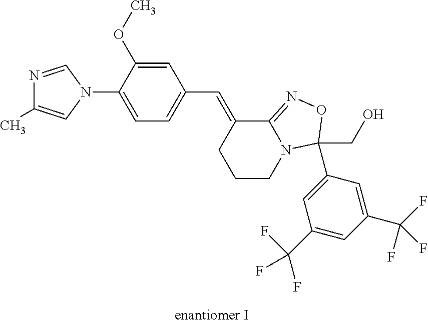 | 3.2 | 567 | C |
| 320 | 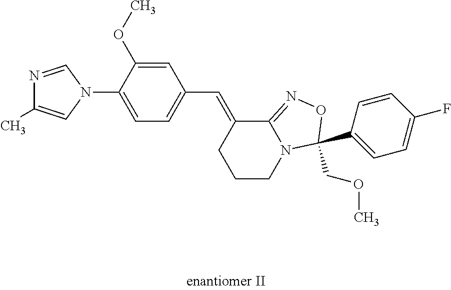 | 3.1 | 463 | D |
| 321 | 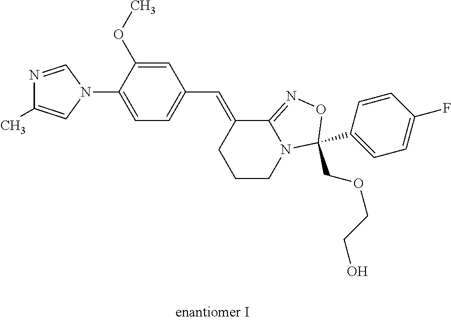 | 2.4 | 493 | D |
| 322 | 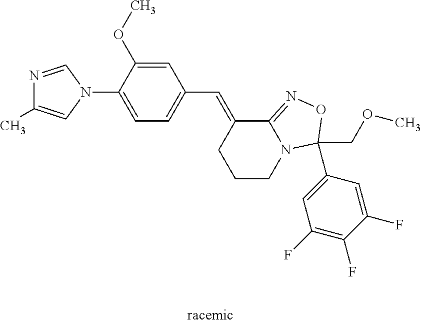 | 3 | 499 | D |
| 323 | 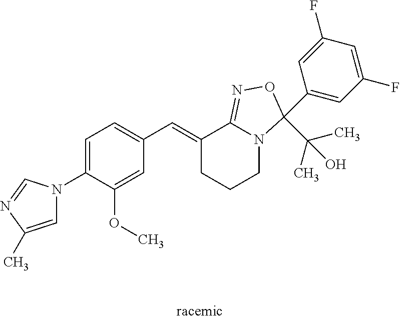 | 2.02(A) | 495 | F |
| 324 | 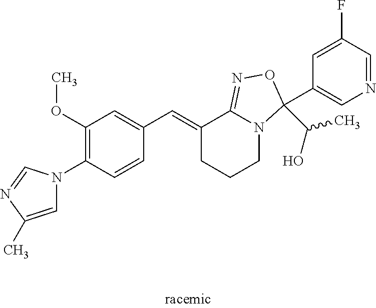 | 1.76 | 464 | H |
| 325 | 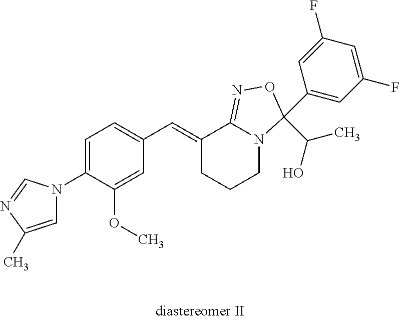 | 3.34(B) | 481 | H |
| 326 | 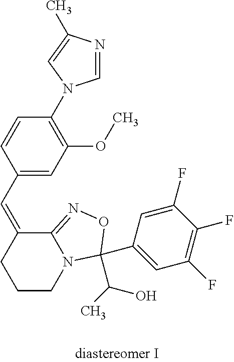 | 2.9 | 499 | H |
| 327 | 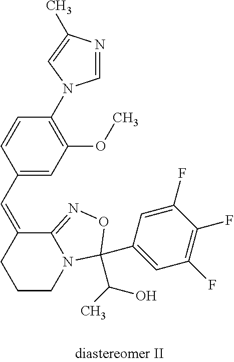 | 2.9 | 499 | H |
| 328 | 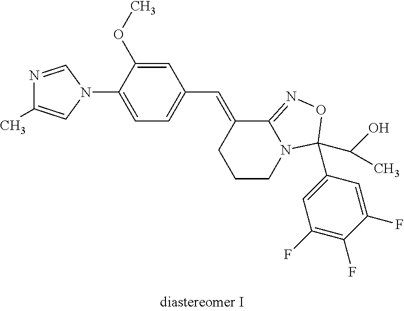 | 3.4 | 499 | H |
| 329 | 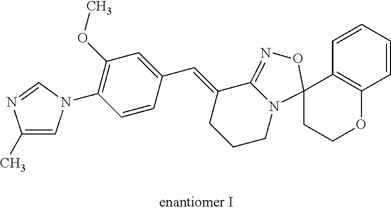 | 3.2 | 443 | I |
| 330 | 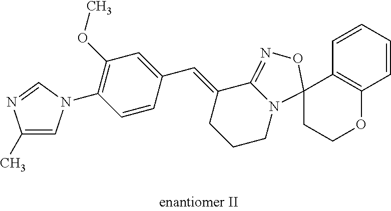 | 3.2 | 443 | I |
| 331 | 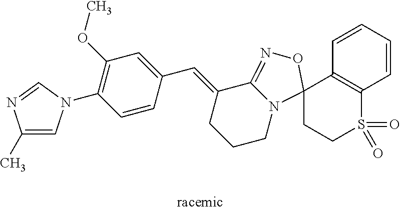 | 3 | 491 | I |
| 332 | 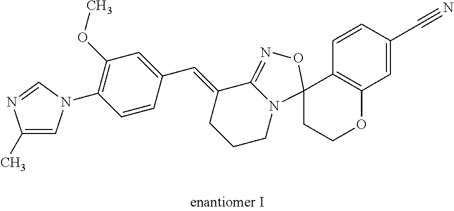 | 3.2 | 468 | I |
| 333 | 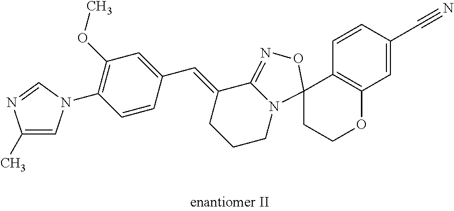 | 3.2 | 468 | I |
| 334 | 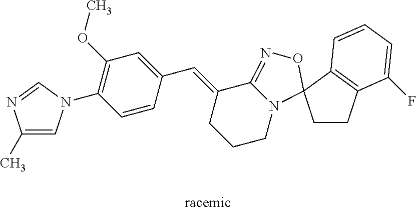 | 3.3 | 445 | I |
| 335 | 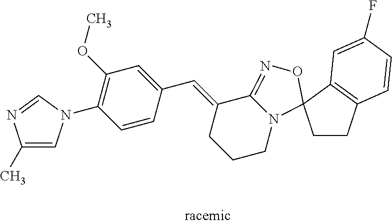 | 3.3 | 445 | I |
| 336 | 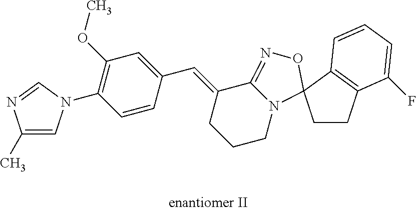 | 3.3 | 445 | I |
| 337 | 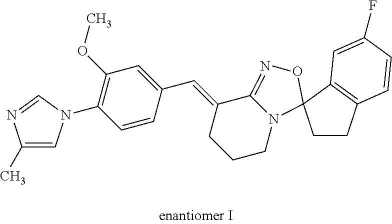 | 3.2 | 445 | I |
| 338 | 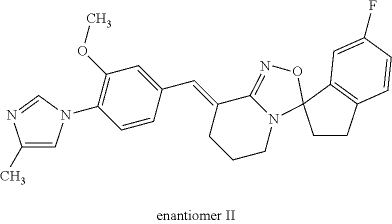 | 3.2 | 445 | I |
| 339 | 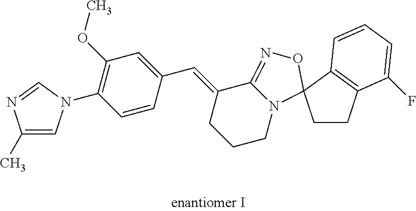 | 3.3 | 445 | I |
| 340 | 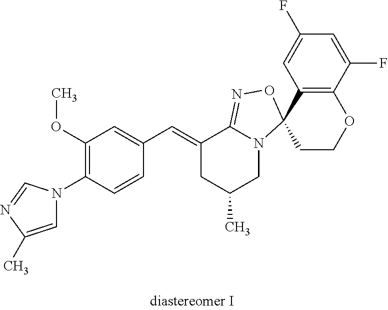 | 3.2 | 493 | I |
| 341 | 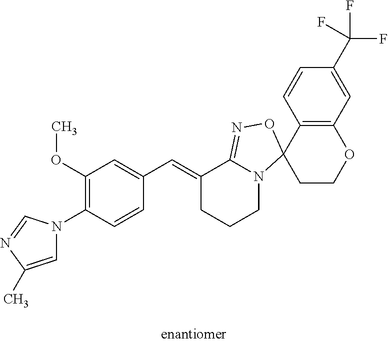 | 3.8 | 511 | I |
| 342 | 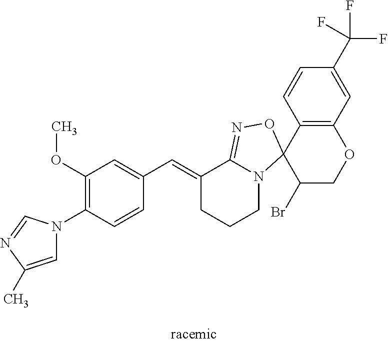 | 3.4 | 590 | I |
| 343 | 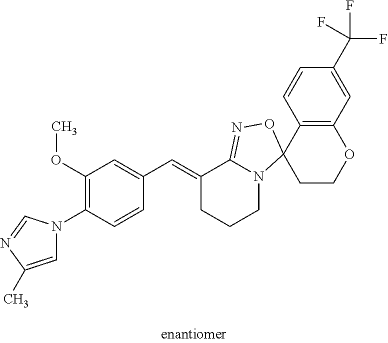 | 3.3 | 511 | I |
| 344 | 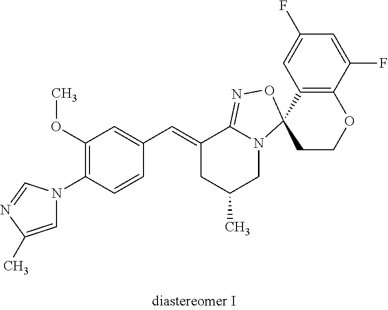 | 3.7 | 493 | I |
| 345 | 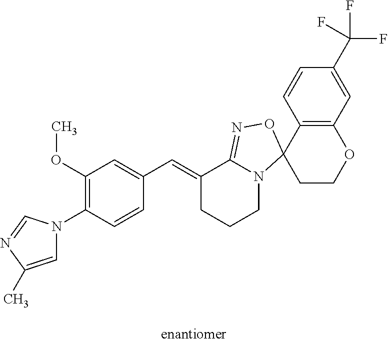 | 3.8 | 511 | I |
| 346 | 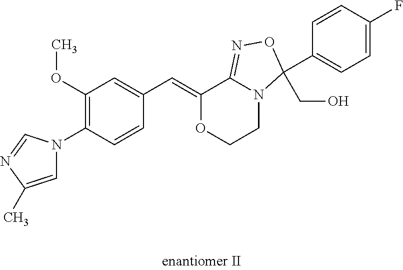 | 2.5 | 451 | J |
| 347 | 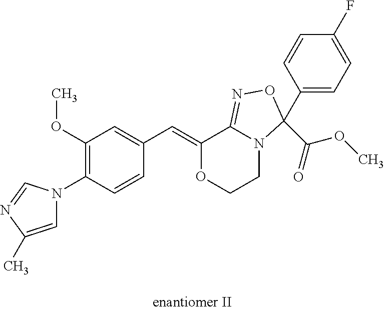 | 3.22 | 479 | J |
| 348 | 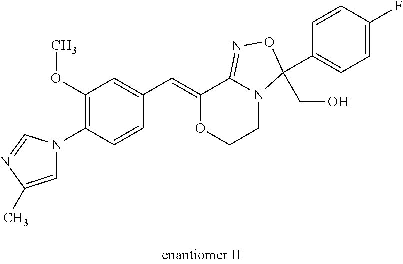 | 2.5 | 451 | J |
| 349 | 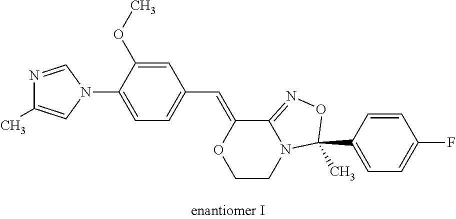 | 3.2 | 435 | J |
| 350 | 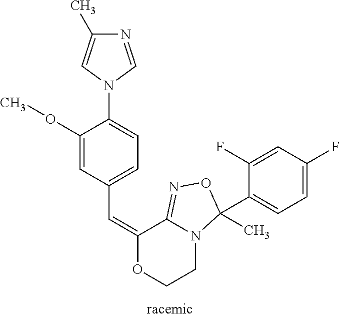 | 2 | 453 | J |
| 351 | 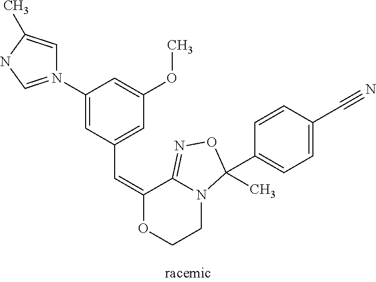 | 3 | 442 | J |
| 352 | 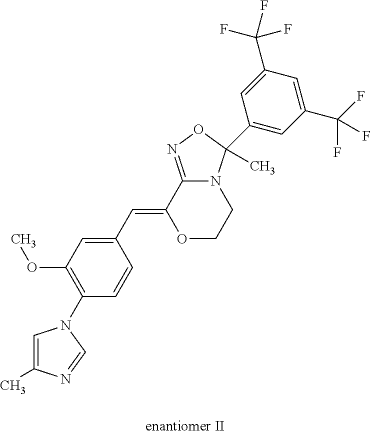 | 3.5 | 563 | J |
| 353 | 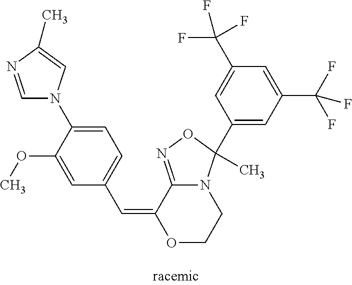 | 3.6 | 553 | J |
| 354 | 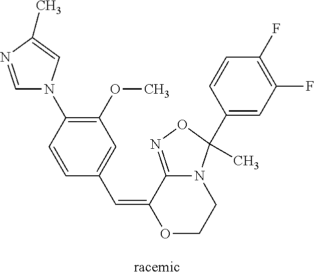 | 3.3 | 453 | J |
| 355 | 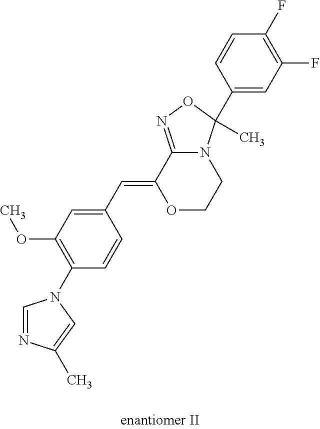 | 2.7 | 453 | J |
| 356 | 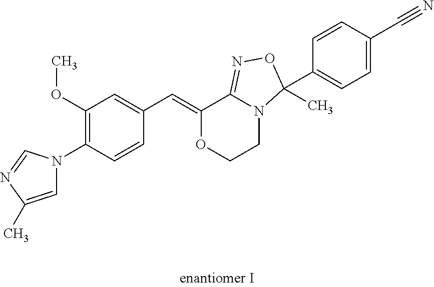 | 3 | 442 | J |
| 357 | 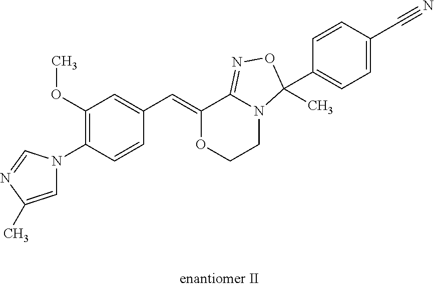 | 3 | 442 | J |
| 358 | 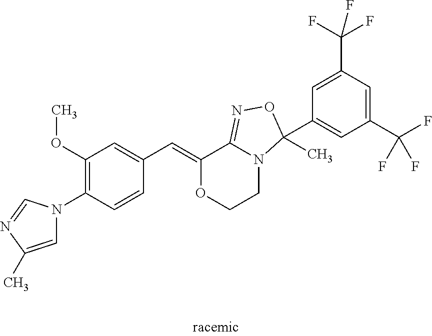 | 3.6 | 553 | J |
| 359 | 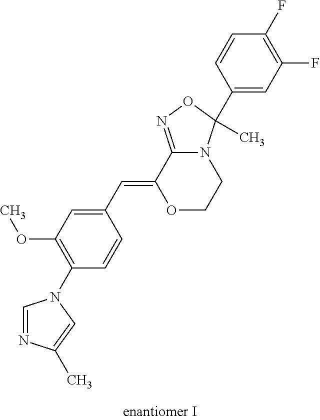 | 2.7 | 453 | J |
| 360 | 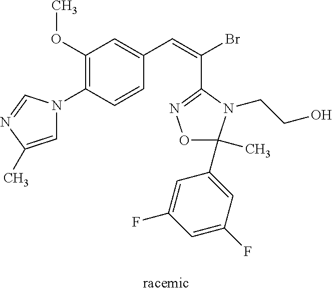 | 1.9 | 535 | J |
| 361 | 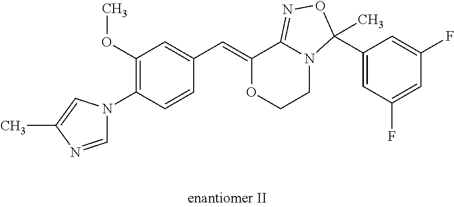 | 3 | 453 | J |
| 362 | 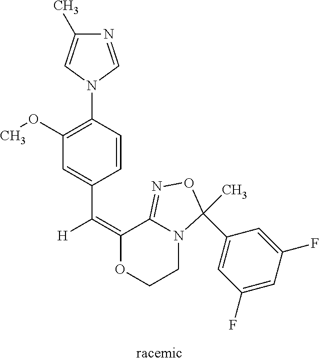 | 3.1 | 453 | J |
| 363 | 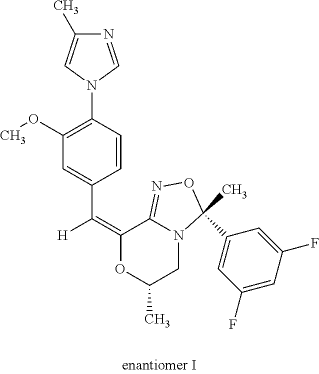 | 3.4 | 467 | J |
| 364 | 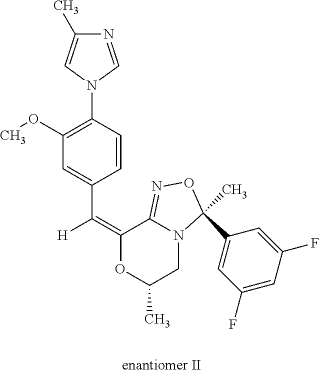 | 3.4 | 467 | J |
| 365 | 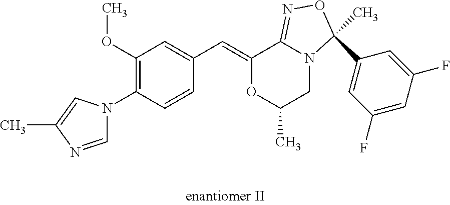 | 3.4 | 467 | J |
| 366 | 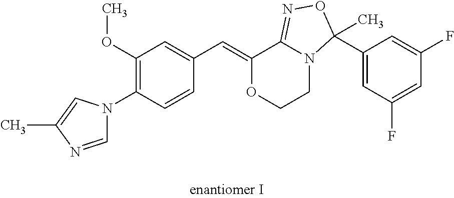 | 3.2 | 453 | J |
| 367 | 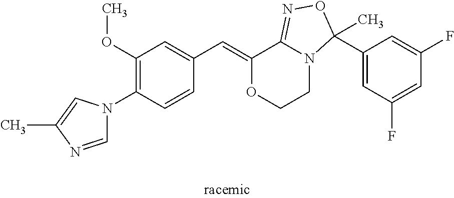 | 3.1 | 453 | J |
| 368 | 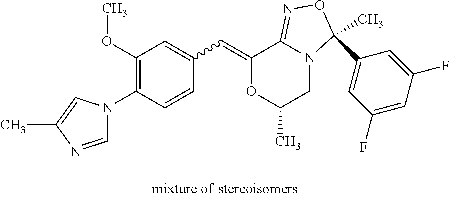 | 2.8 | 467 | J |
| 369 | 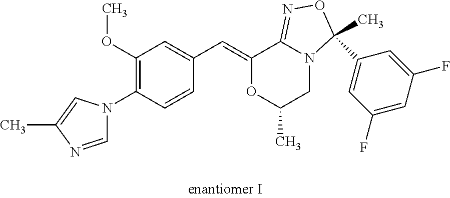 | 3.3 | 467 | J |
| 370 | 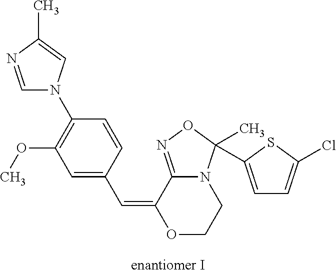 | 3.3 | 467 | J |
| 371 | 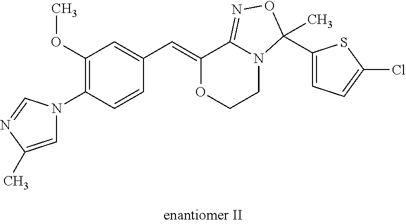 | | | J |
| 372 | 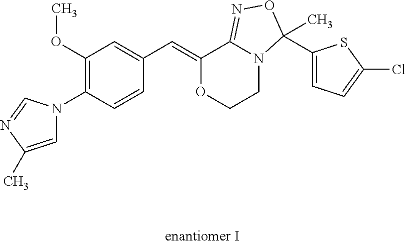 | 3.4 | 457 | J |
| 373 | 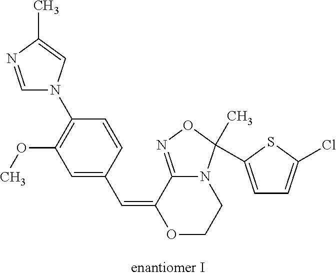 | 3.3 | 457 | J |
| 374 | 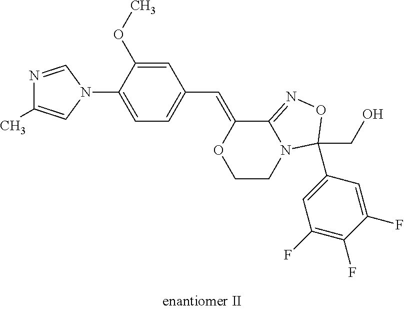 | 3 | 487 | J |
| 375 | 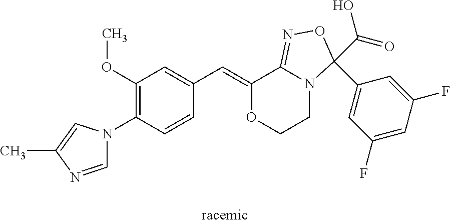 | 2.8 | 483 | J |
| 376 | 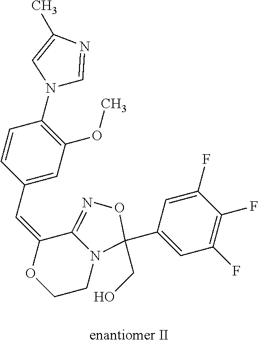 | 3.1 | 487 | J |
| 377 | 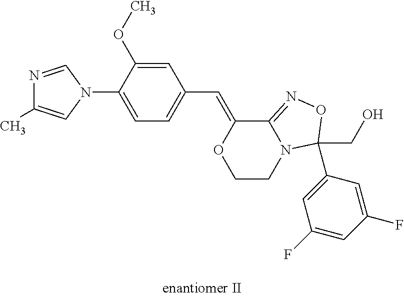 | 2.9 | 469 | J |
| 378 | 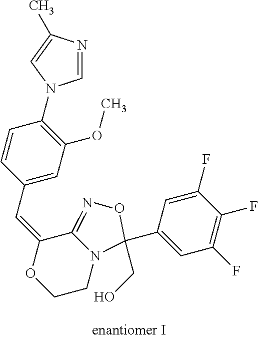 | 3.1 | 487 | J |
| 379 | 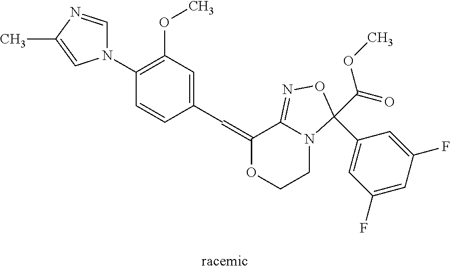 | 3.2 | 497 | J |
| 380 | 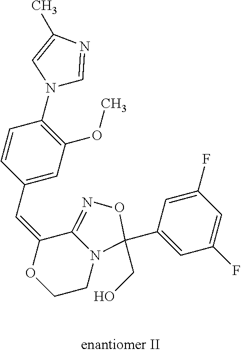 | 2.9 | 469 | J |
| 381 | 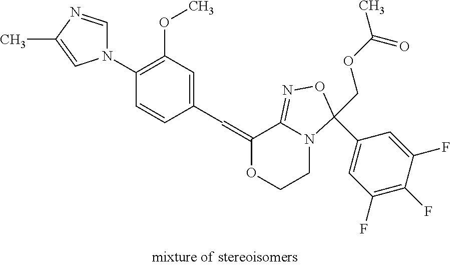 | 3.4 | 529 | J |
| 382 | 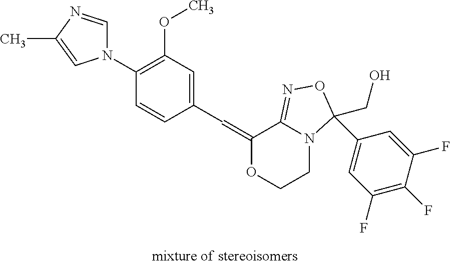 | 3.1 | 487 | J |
| 383 | 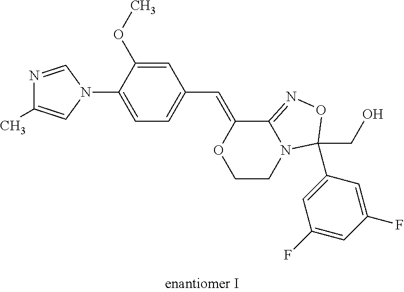 | 2.9 | 469 | J |
| 384 | 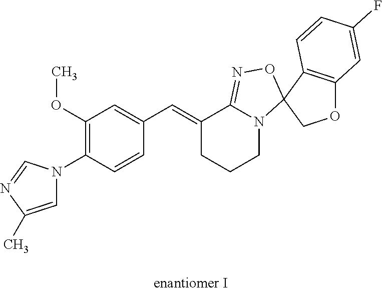 | 3.1 | 447 | P |
| 385 | 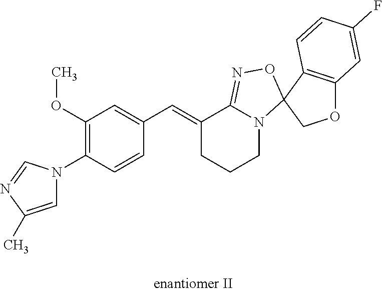 | 3.3 | 447 | P |
| 386 | 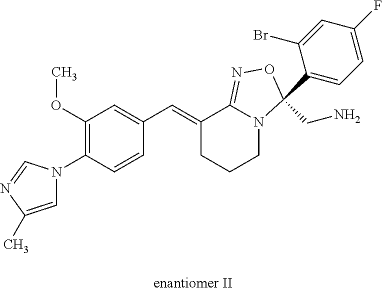 | 2.2 | 527 | Q |
| 387 | 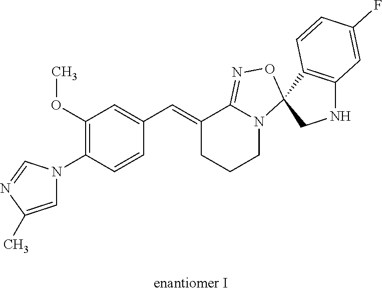 | 2.9 | 446 | Q |
| 388 | 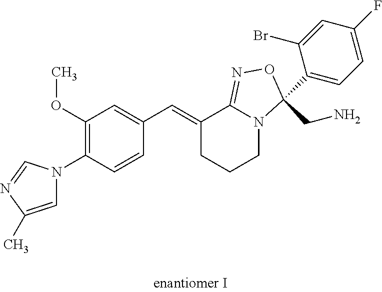 | 2.5 | 527 | Q |
| 389 | 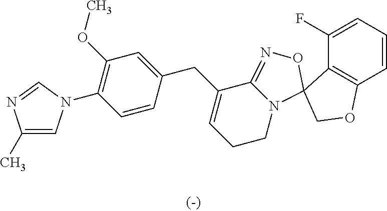 | 3.1 | 447 | R |
| 390 | 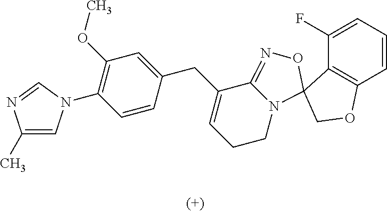 | 3.1 | 447 | R |
| 391 | 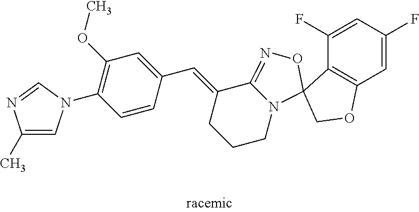 | 3 | 465 | R |
| 392 | 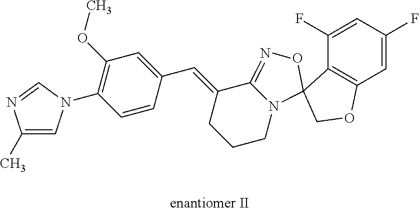 | 3 | 465 | R |
| 393 | 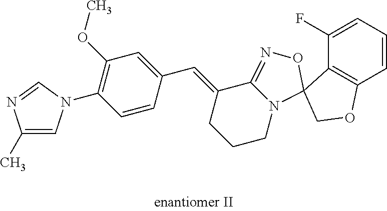 | 2.9 | 447 | R |
| 394 | 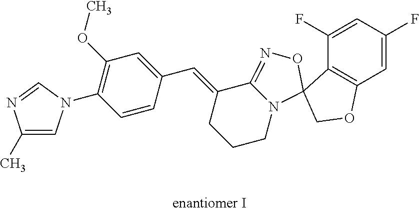 | 3.3 | 465 | R |
| 395 | 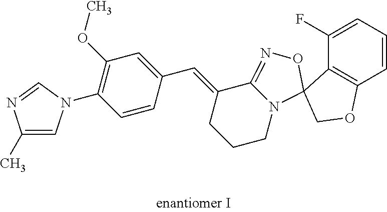 | 3.2 | 447 | R |
| 396 | 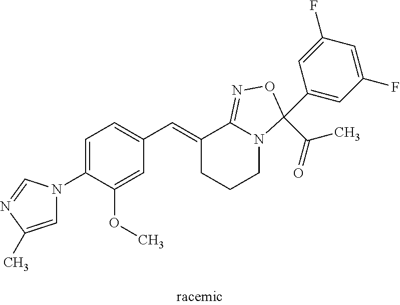 | 2.06(A) | 479 | S |
| 397 | 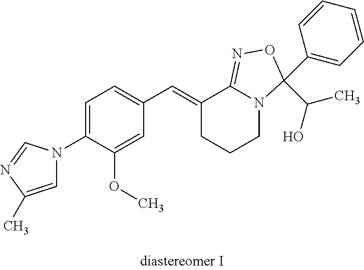 | 3.1 | 445 | T |
| 398 | 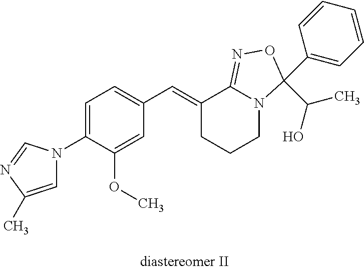 | 3.011(B) | 445 | T |
| 399 | 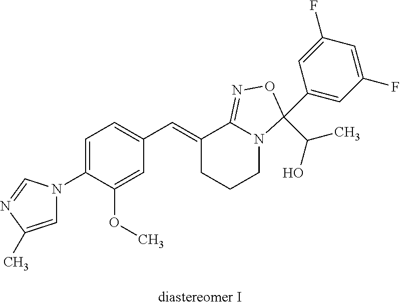 | 3.22(B) | 481 | T |
| 400 | 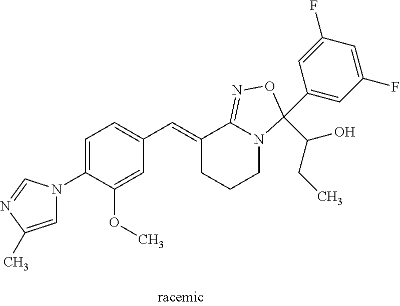 | 2.002(A) | 495 | U |
| 401 | 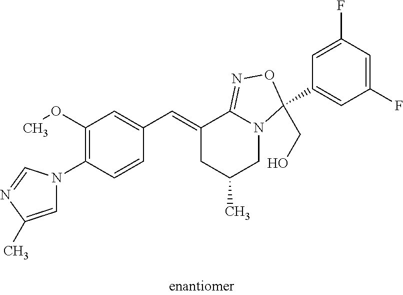 | 1.97(A) | 483 | V |
| 402 | 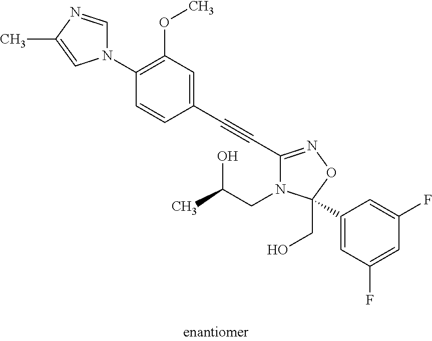 | 1.91(A) | 483 | W |
| 403 | 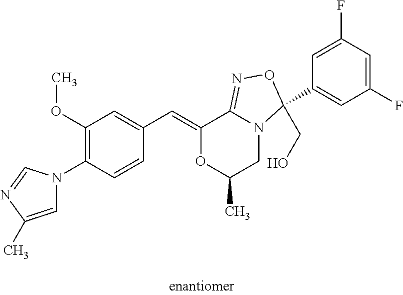 | 1.98(A) | 483 | W |
| 404 | 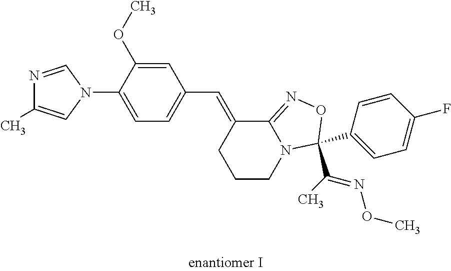 | 3.4 | 490 | X |
| 405 | 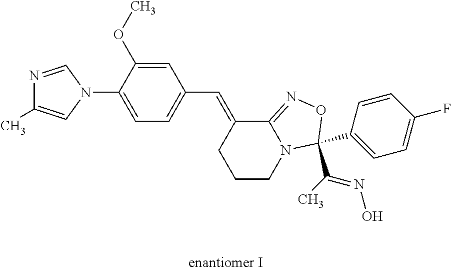 | 3 | 476 | X |
| 406 | 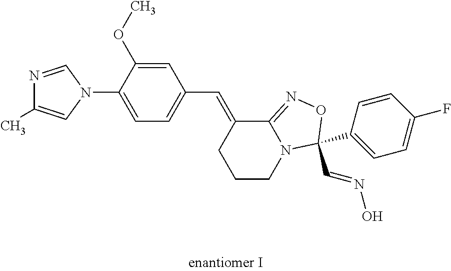 | 3 | 462 | X |
| 407 | 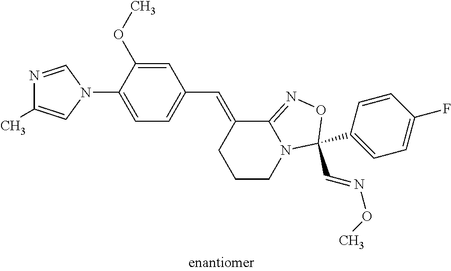 | 3.4 | 476 | X |
| 408 | 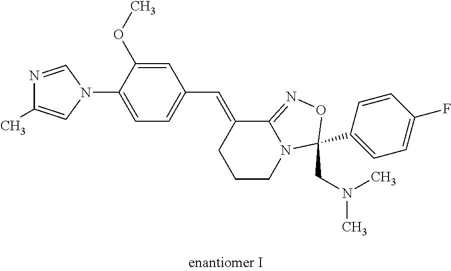 | 2.3 | 476 | Y |
| 409 | 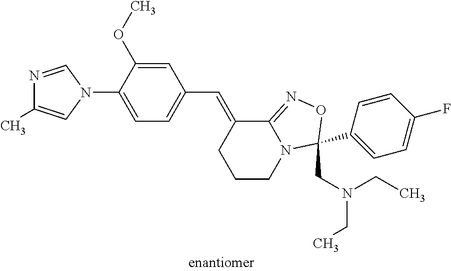 | 2.5 | 504 | Y |
| 410 | 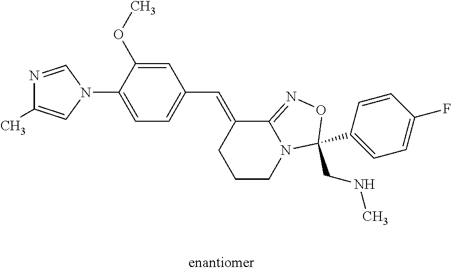 | 2.4 | 462 | Y |
| 411 | 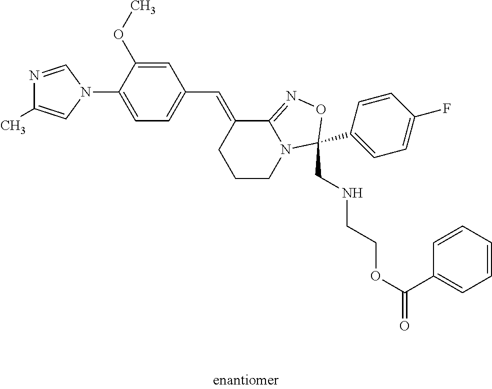 | 2.7 | 596 | Y |
| 412 | 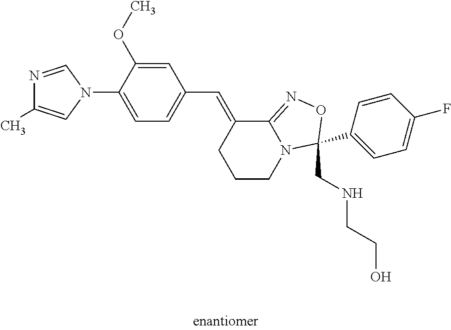 | | | Y |
| 413 | 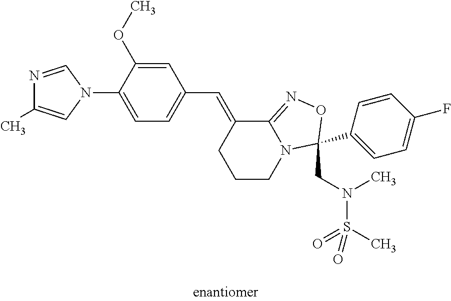 | 3 | 540 | Z |
| 414 | 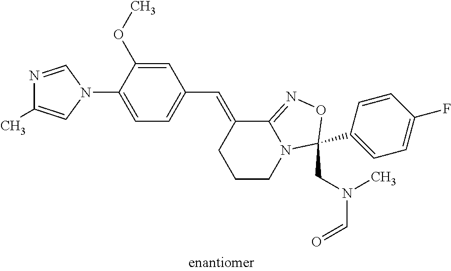 | 2.8 | 490 | Z |
| 415 | 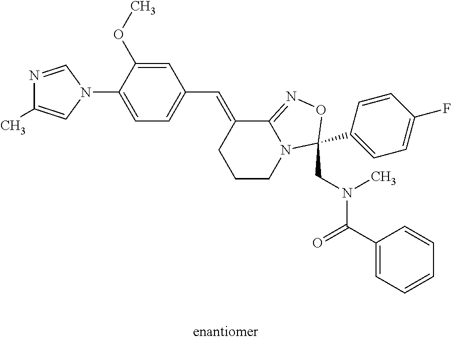 | 3.2 | 566 | Z |
| 416 | 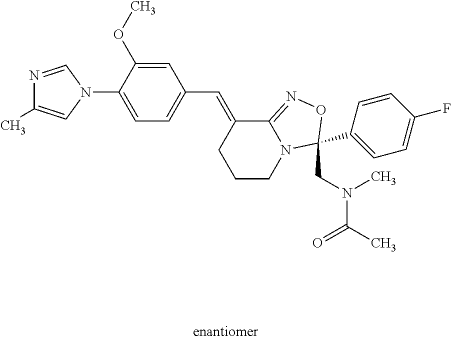 | 2.9 | 504 | Z |
| 417 | 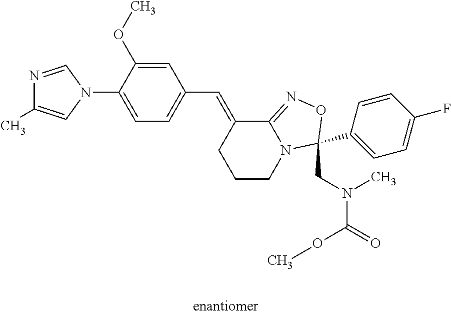 | | | Z |
| 418 | 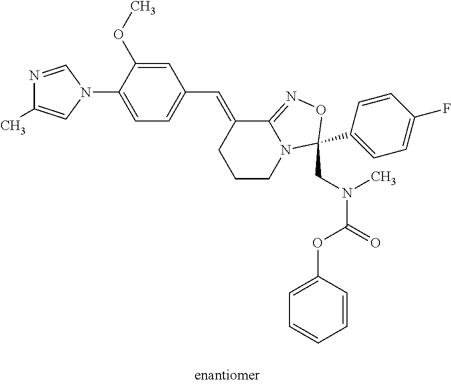 | | | Z |
| 419 | 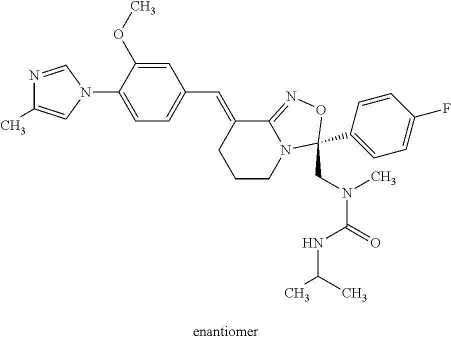 | | | Z |
| 420 | 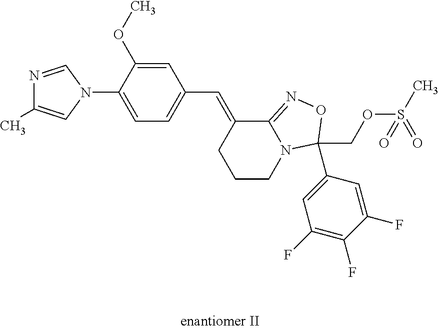 | 3.3 | 563 | Z |

The following compounds will be synthesized using method similar to that listed in the synthetic method column.

| | | | Obs | Synthetic |
|---|---|---|---|---|
| # | Structure | Rt (min) | Mass | Method |
| 421 | 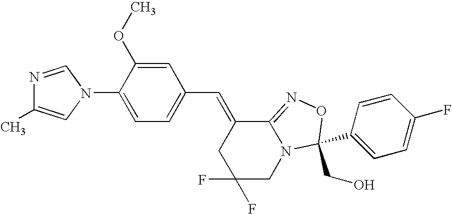 | | | AC |
| 422 | 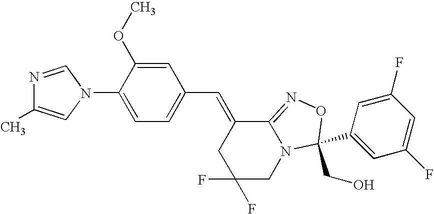 | | | AC |
| 423 | 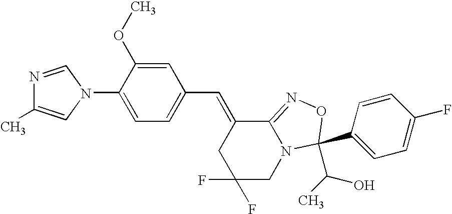 | | | AC |
| 424 | 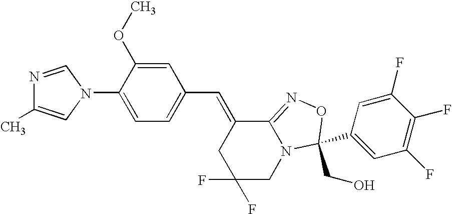 | | | AC |
| 425 | 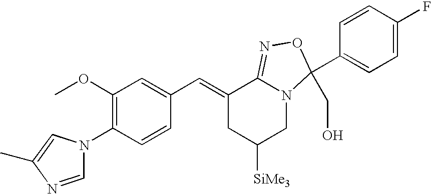 | | | AF |
| 428 | 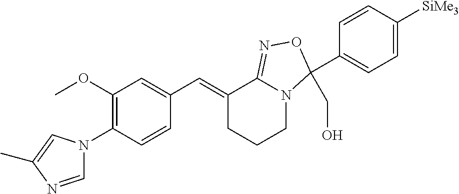 | | | AF |
| 434 | 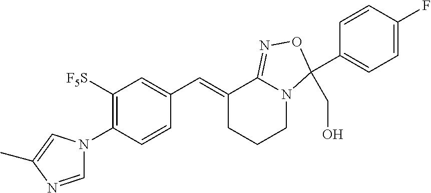 | | | AF |
| 435 | 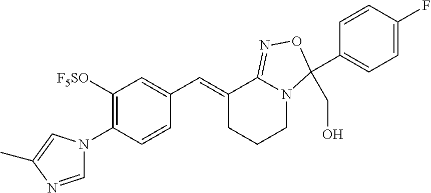 | | | AF |
| 436 | 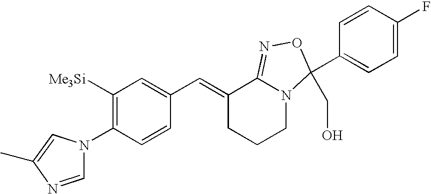 | | | AF |
| 437 | 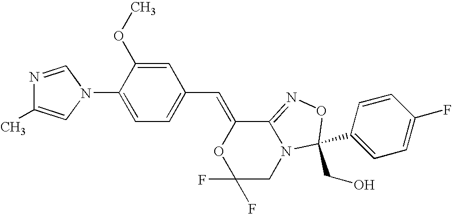 | | | AH |
| 438 | 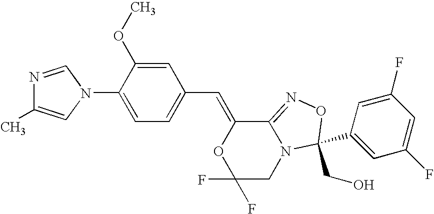 | | | AH |
| 439 | 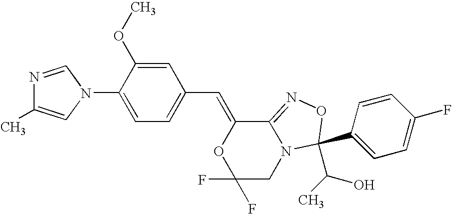 | | | AH |
| 440 | 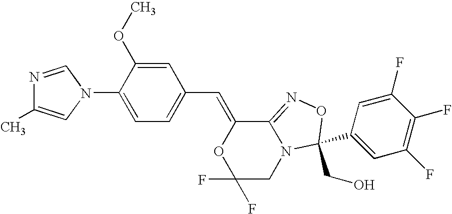 | | | AH |
| 441 | 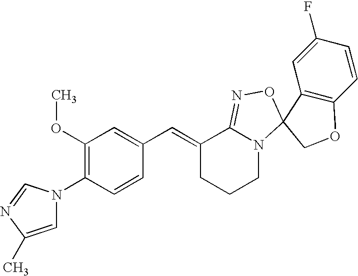 | | | P |
| 442 | 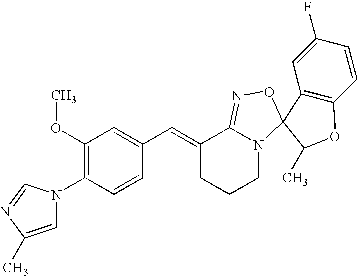 | | | P |
| 443 | 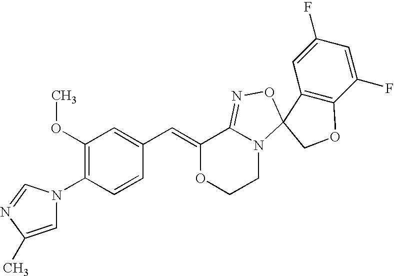 | | | P |
| 444 | 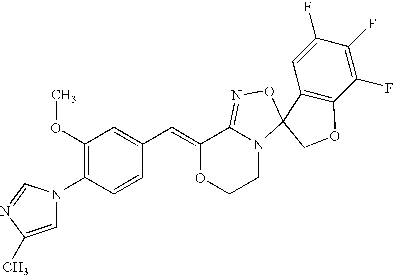 | | | P |
| 445 | 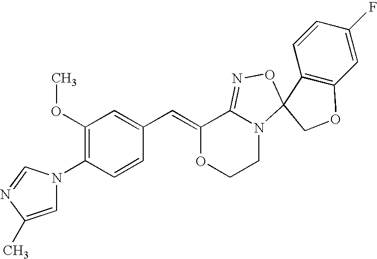 | | | P |
| 446 | 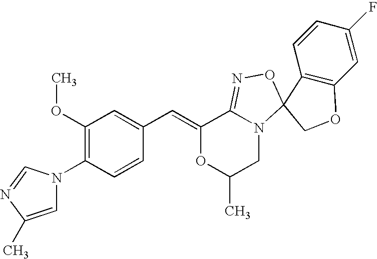 | | | P |

The following compounds were synthesized using a method similar to the method listed in the last column.

| | | Observed | | Synthetic |
|---|---|---|---|---|
| # | Structure | Mass | Rt | Method |
| 447 | 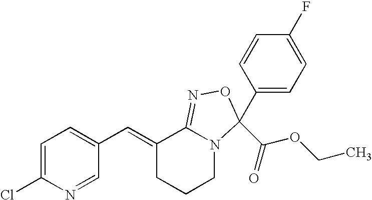 | 416.2 | — | B |
| 448 | 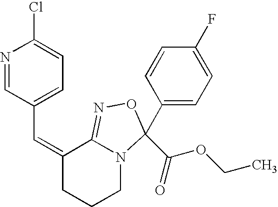 | 416.2 | — | B |
| 449 | 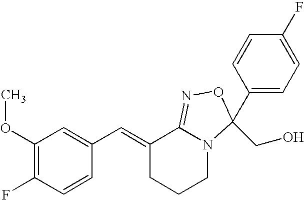 | 387.21 | 4.2 | C |
| 450 | 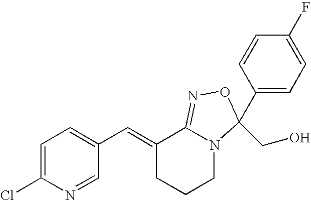 | 374.21 | 4 | C |
| 451 | 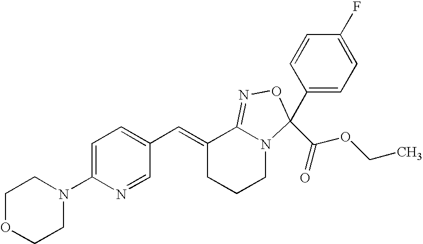 | 467.26 | 3.2 | AK |
| 452 | 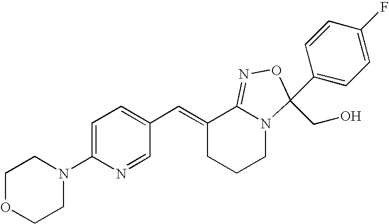 | 425.23 | 2.5 | AK |
| 453 | 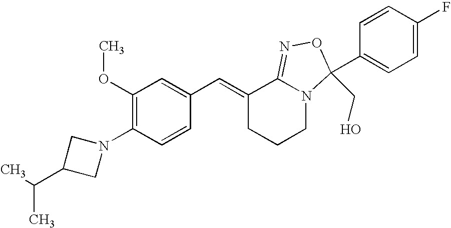 | 467.26 | 4.3 | C |
| 454 | 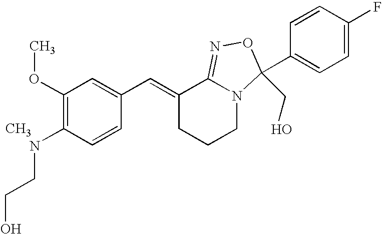 | 442.24 | 2.5 | C |
| 455 | 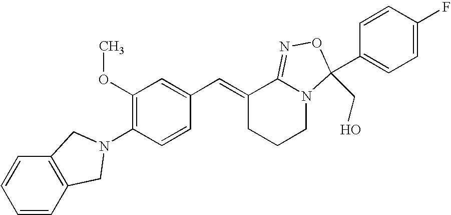 | 486.27 | 4.5 | C |
| 456 | 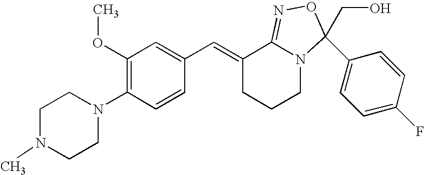 | 467.26 | 4.2 | C |
| 457 | 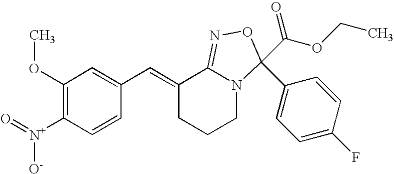 | 456.3 | — | B |
| 458 | 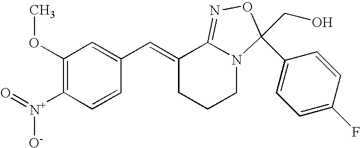 | 414.23 | 4.2 | C |
| 459 | 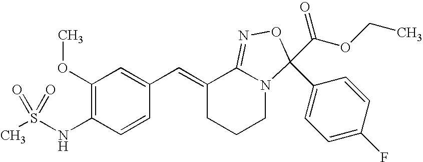 | 504.3 | — | Y |
| 460 | 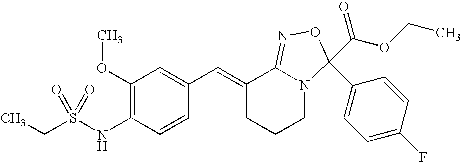 | 518.3 | — | Y |
| 461 | 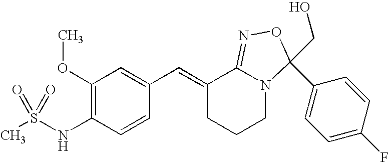 | 462.3 | — | Y |
| 462 | 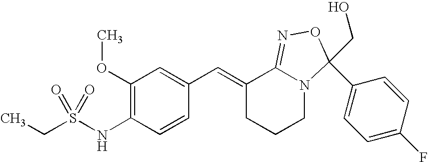 | 476.3 | — | Y |
| 463 | 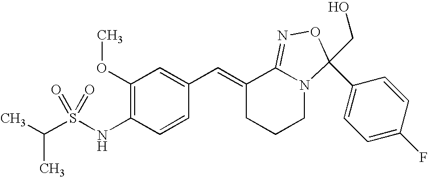 | 490.3 | — | Y |
| 464 | 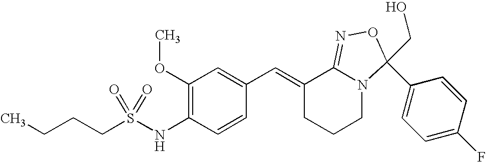 | 504.3 | — | Y |
| 465 | 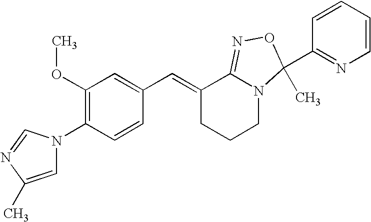 | | | B |
| 468 | 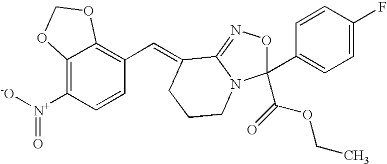 | 470.26 | 5 | B |
| 469 | 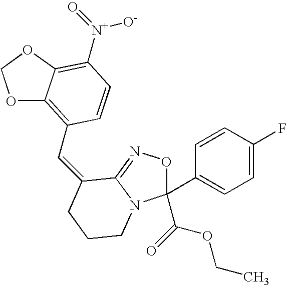 | 470.26 | 4.9 | B |
| 470 | 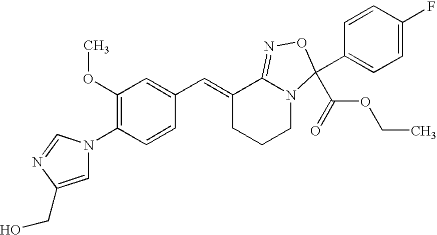 | 507.28 | 3.2 | B |
| 471 | 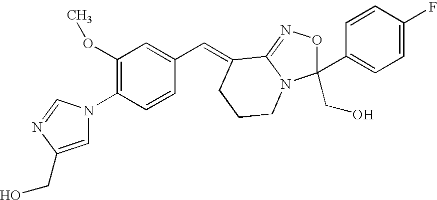 | 465.26 | 2.7 | B |
| 472 | 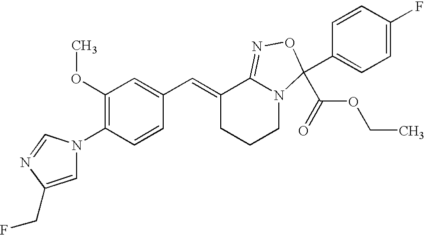 | 509.28 | 3.5 | B |
| 473 | 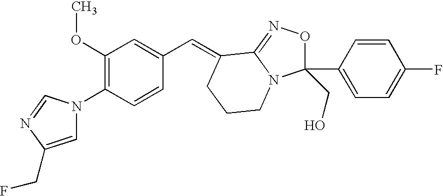 | 467.26 | 3.6 | C |
| 474 | 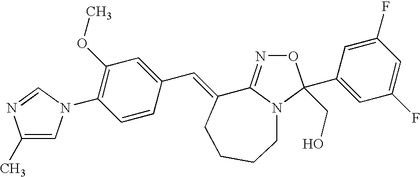 | 481.26 | 2.9 | C |
| 475 | 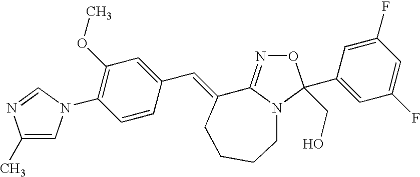 | 481.26 | 2.9 | C |
| 476 | 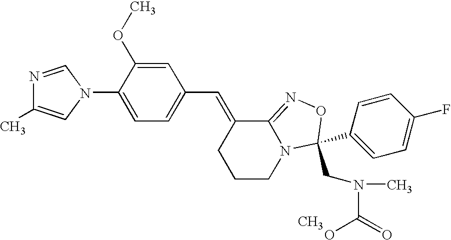 | 520.29 | 3.1 | Y |
| 477 | 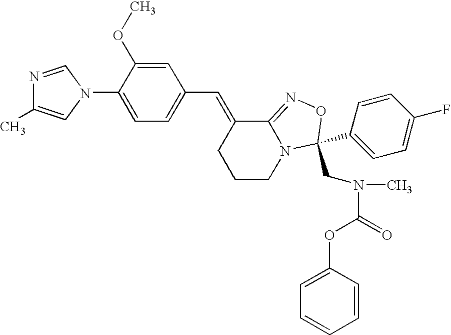 | 582.32 | 3.5 | Y |
| 478 | 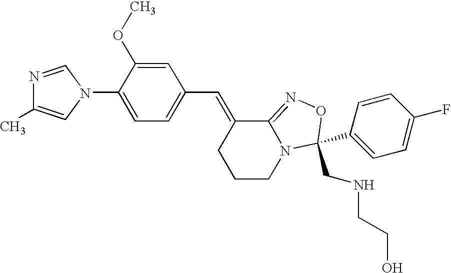 | 492.27 | 2.3 | Y |
| 479 | 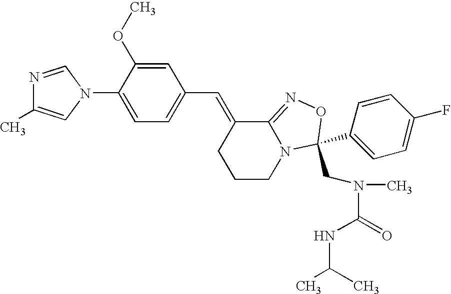 | 547.3 | 3.1 | Y |
| 480 | 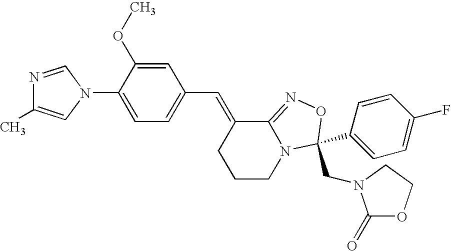 | 518.28 | 3 | Y |
| 481 | 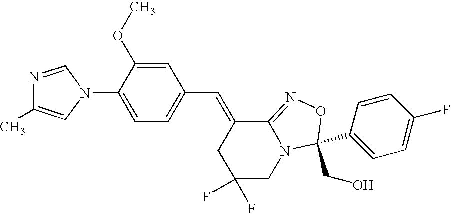 | 485.27 | 2.9 | AC |
| 482 | 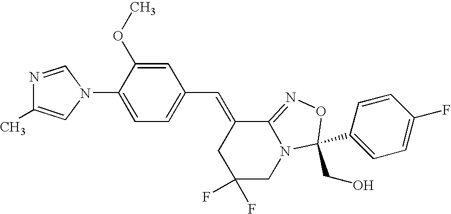 | 485.27 | 2.9 | AC |
| 483 | 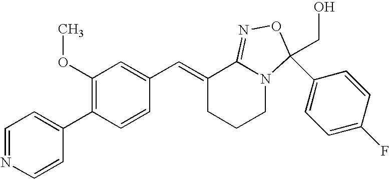 | 446.25 | 2.9 | AJ |
| 484 | 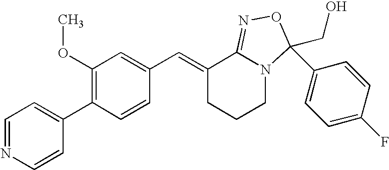 | 446.25 | 2.8 | AJ |
| 485 | 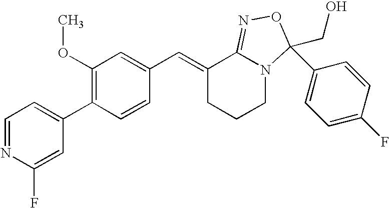 | 464.26 | 5.4 | AJ |
| 486 | 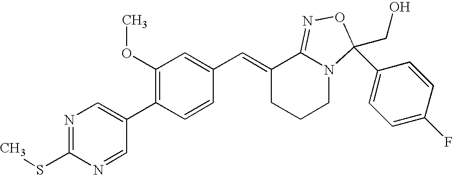 | 493.3 | — | AJ |
| 487 | 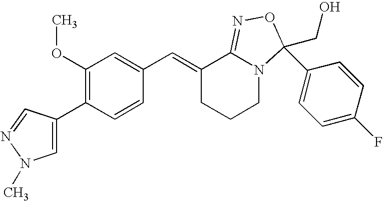 | 449.25 | 5.2 | AJ |
| 488 | 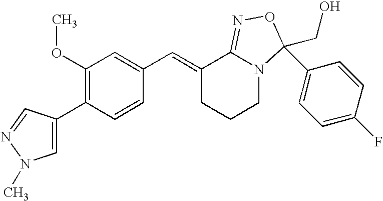 | 449.25 | — | AJ |
| 489 | 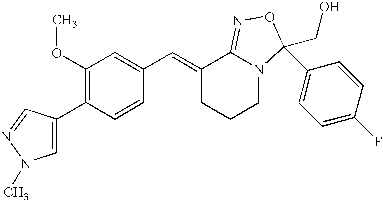 | 449.25 | — | AJ |
| 490 | 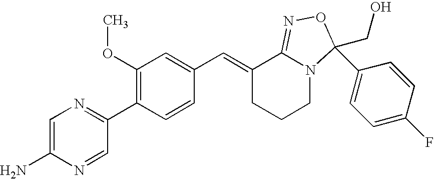 | 462.25 | 3.8 | AJ |
| 491 | 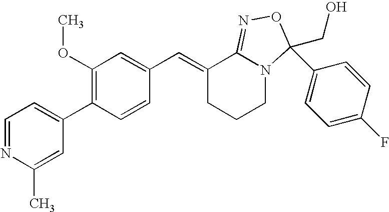 | 460.25 | 3.8 | AJ |
| 492 | 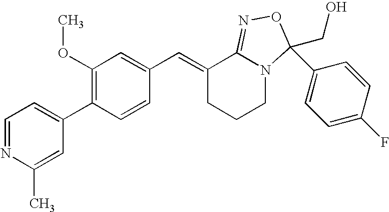 | 460.25 | 3.8 | AJ |
| 493 | 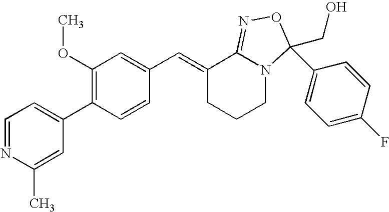 | 460.25 | — | AJ |
| 494 | 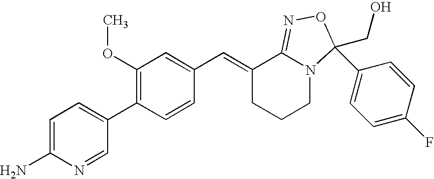 | 461.25 | 3.7 | AJ |
| 495 | 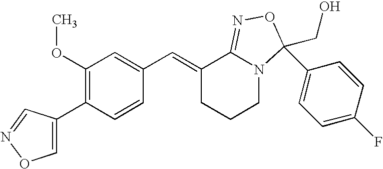 | 436.24 | 4.3 | AJ |
| 496 | 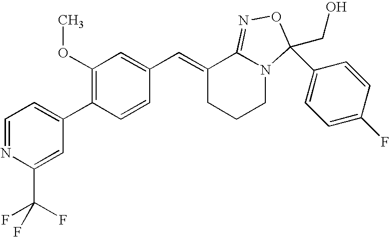 | 514.28 | 5.9 | AJ |
| 497 | 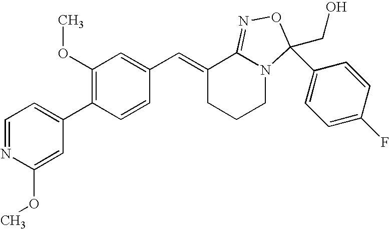 | 476.26 | 2.7 | AJ |
| 498 | 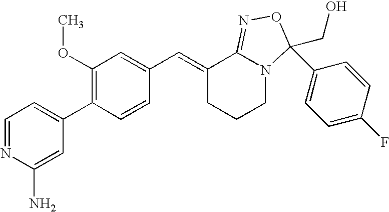 | 461.25 | 2.2 | AJ |
| 499 | 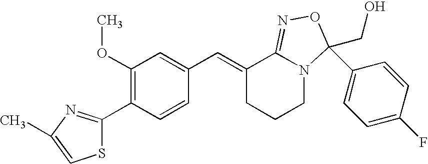 | 466.26 | 4.9 | AJ |
| 500 | 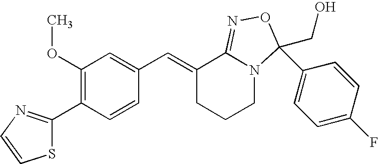 | 452.25 | 3 | AJ |
| 501 | 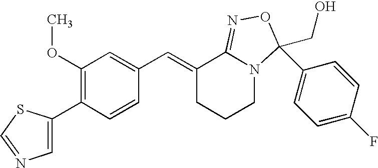 | 452.25 | 5 | AJ |
| 502 | 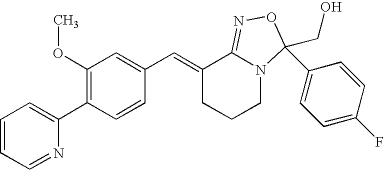 | 446.25 | 2.1 | AJ |
| 503 | 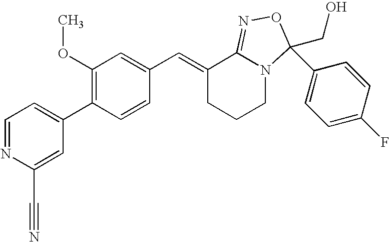 | 471.26 | 3 | AJ |
| 504 | 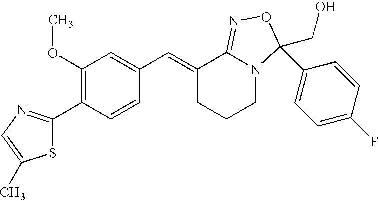 | | | AJ |
| 505 | 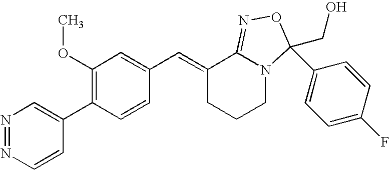 | | | AJ |
| 506 | 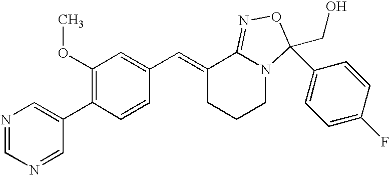 | | | AJ |
| 507 | 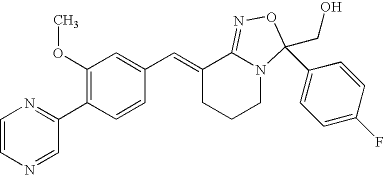 | | | AJ |
| 508 | 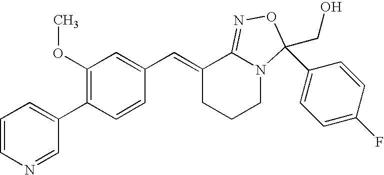 | | | AJ |
| 509 | 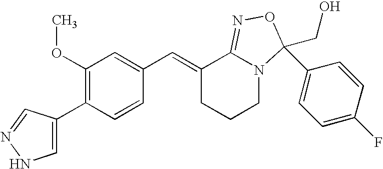 | | | AJ |
| 510 | 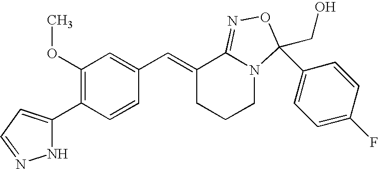 | | | AJ |
| 511 | 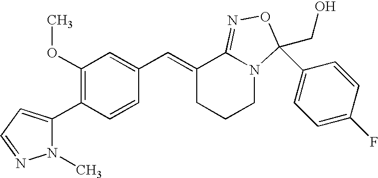 | | | AJ |
| 512 | 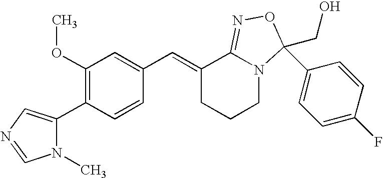 | | | AJ |
| 513 | 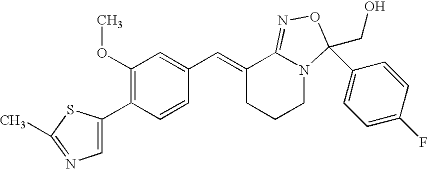 | | | AJ |
| 514 | 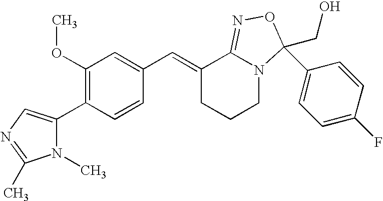 | | | AJ |
| 515 | 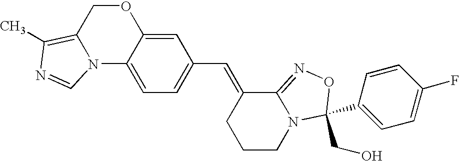 | 447.25 | 3.9 | C |
| 516 | 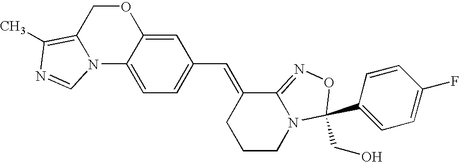 | 447.25 | 3.9 | C |
| 517 | 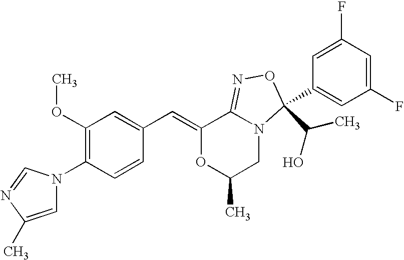 | 497.27 | 4 | J, U |
| 518 | 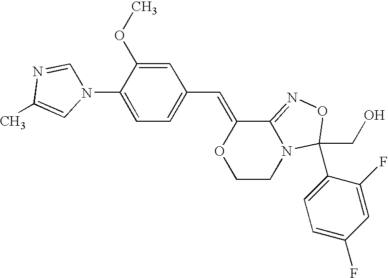 | 469.26 | 2.9 | J, U |
| 519 | 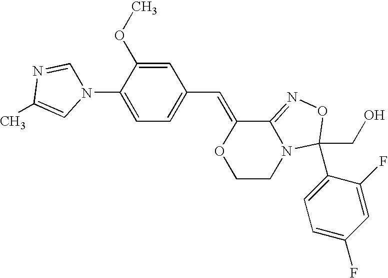 | 469.26 | 2.9 | J |
| 520 | 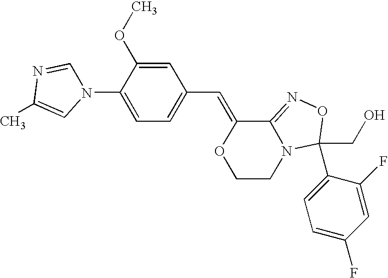 | 469.26 | 2.9 | J |
| 521 | 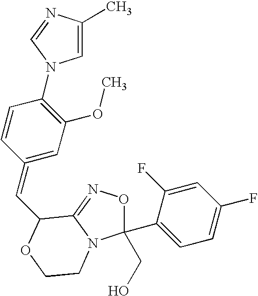 | 469.26 | 3 | J |
| 522 | 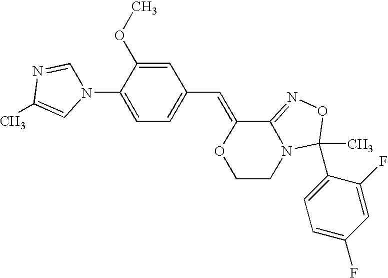 | 453.25 | 3.3 | J |
| 523 | 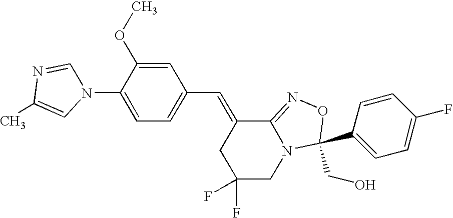 | 485.27 | 3 | AC |
| 524 | 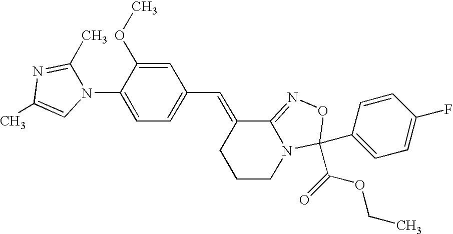 | 505.28 | 3.4 | B |
| 525 | 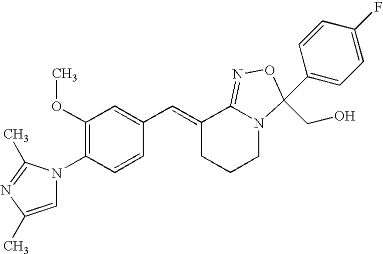 | 463.25 | 3 | C |
| 526 | 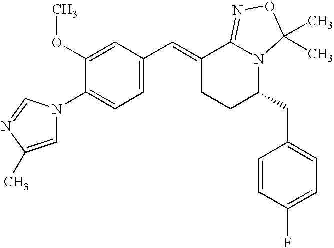 | 461.25 | 4.3 | B |
| 527 | 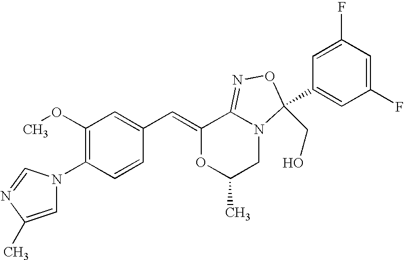 | 483 | — | C |
| 528 | 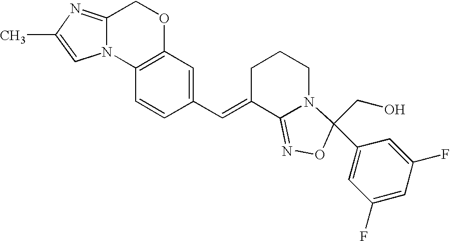 | 465 | — | C |
| 529 | 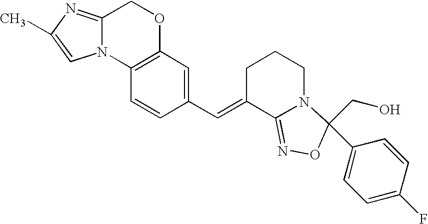 | 447 | — | C |
| 530 | 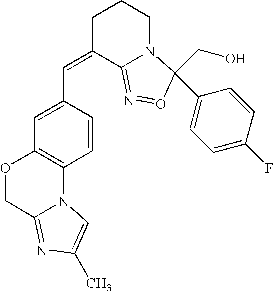 | | | C |
| 531 | 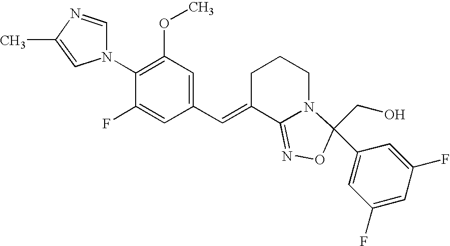 | | | B |
| 532 | 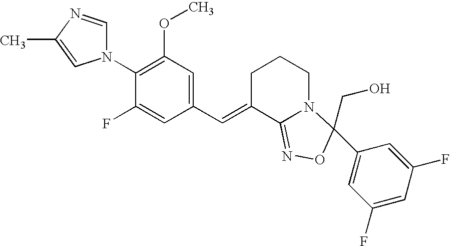 | | | B |
| 533 | 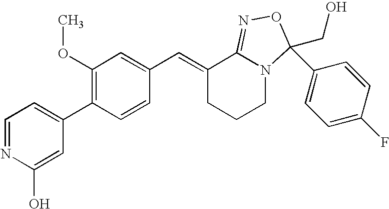 | 463.2 | 1.9 | AJ |

### Assay:

Secretase Reaction and Aβ Analysis in Whole Cells: HEK293 Cells overexpressing APP with Swedish and London mutations were treated with the specified compounds for 5 hour at 37° C. in 100 ml of DMEM medium containing 10% fetal bovine serum. At the end of the incubation, total Aβ, Aβ40 and Aβ42 were measured using electrochemiluminescence (ECL) based sandwich immunoassays. Total Aβ was determined using a pair of antibodies TAG-W02 and biotin-4G8, Aβ40 was identified with antibody pairs TAG-G2-10 and biotin-4G8, while Aβ42 was identified with TAG-G2-11 and biotin-4G8. The ECL signal was measured using Sector Imager 2400 (Meso Scale Discovery).

MS Analysis of Aβ Profile: Aβ profile in conditioned media was determined using surface enhanced laser desorption/ionization (SELDI) mass spectrometry. Conditioned media was incubated with antibody W02 Coated PS20 ProteinChip array. Mass spectra of Aβ captured on the array were read on SELDI ProteinChip Reader (Bio-Rad) according to manufacture's instructions.

CSF Aβ Analysis: Aβ in rat CSF was determined using MSD technology as described above. Aβ40 was measured using antibody pair Tag-G2-10 and biotin-4G8, while Aβ42 was measured using Tag-anti Aβ42 (Meso Scale Discovery) and biotin-4G8. The ECL signal was measured using Sector Imager 2400 (Meso Scale Discovery).

MS analysis of Aβ profile: To isolate Aβ products from conditioned media, cells expressing APP were grown to 90% confluence and re-fed with fresh media containing γ-secretase modulator. The conditioned media, harvested after 16 h of incubation, were incubated overnight with antibody W02 in RIPA buffer (20 mM Tris-HCl, pH7.4, 150 mM NaCl, 0.2% Twenn 20, 0.2% Triton 100 and 0.2% NP40). Protein A plus G agarose (Calbiochem) was added to the reaction and the mixture was rocked at room temperature for another 2 h. The agarose beads were then collected by centrifugation and washed 3 times with RIPA buffer and twice with 20 mM Tris (pH 7.4). The immunoprecipitated peptides were eluted from the beads with 10 μL of 10% acetonitrile/0.1% trifluoroacetic acid (TFA).

Matrix-assisted laser desorption/ionization mass spectrometric (MALDI MS) analysis of Aβ was performed on a Voyager-DE STR mass spectrometer (ABI, Framingham, Mass.). The instrument is equipped with a pulsed nitrogen laser (337 nm). Mass spectra were acquired in the linear mode with an acceleration voltage of 20 kV. Each spectrum presented in this work represents an average of 256 laser shots. To prepare the sample-matrix solution, 1 μL of immunoprecipitated Aβ sample was mixed with 3 μL of saturated α-cyano-4-hydroxycinnamic acid solution in 0.1% TFA/acetonitrile. The sample-matrix solution was then applied to the sample plate and dried at ambient temperature prior to mass spectrometric analysis. All the spectra were externally calibrated with a mixture of bovine insulin and ACTH (18-39 Clip).

Certain compounds of this invention had an Aβ42 IC50 in the range of about 14 nM to about 16,462 nM. Certain compounds of this invention had an Aβ42 IC50 in the range of about 14 nM to about 1000 nM. Certain compounds of this invention had an Aβ42 IC50 in the range of about 14 nM to about 591 nM. Certain compounds of this invention had an Aβ42 IC50 in the range of about 14 nM to about 101 nM.

Certain compounds of this invention had an Abtotal/Aβ42 IC50 ratio from about 1 to about 1012. Certain compounds of this invention had an Abtotal/Aβ42 IC50 ratio from about 101 to about 1012. Certain compounds of this invention had an Abtotal/Aβ42 IC50 ratio from about 503 to about 1012.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1-15. (canceled)

16. A compound selected from the group consisting of: compounds P2, Q3, R2, S3, T2, U2, V8, W6, X2, X3, Y2, Z2, AA2, AA3, AB2, AC12, AD7, AE4, AG2, AH7, AI2, AJ12, 201-214, 216-266, 268-424, 437-465, and 468-553, or a pharmaceutically acceptable salt thereof.

17. A compound selected from the group consisting of: compounds P2, Q3, R2, S3, T2, U2, V8, W6, X2, X3, Y2, Z2, AA2, AA3, AB2, AC12, AD7, AE4, AG2, AH7, 201-214, 216-266, 268-424, and 437-446.

18. A pharmaceutically acceptable salt of a compound of claim 16.

19. A pharmaceutical composition comprising a therapeutically effective amount of a compound of claim 16 or a pharmaceutically acceptable salt thereof, and a pharmaceutically acceptable carrier.

20. A method of modulating gamma-secretase comprising administering an effective amount of one or more compounds of claim 16 or a pharmaceutically acceptable salt thereof to a patient in need of such treatment.

21. A method of inhibiting the deposition of amyloid protein in, on or around neurological tissue, comprising administering an effective amount of one or more compounds of claim 16 or a pharmaceutically acceptable salt thereof to a patient in need of treatment.

22. A method of treating Alzheimer's disease, comprising administering an effective amount of one or more compounds of claim 16 or a pharmaceutically acceptable salt thereof to a patient in need of treatment.
